(19) United States
(12) Patent Application Publication
(10) Pub. No.: US 2025/0064833 A1
(43) Pub. Date: Feb. 27, 2025

(54) COMBINATION OF MUSCARINIC RECEPTOR POSITIVE MODULATORS AND NMDA POSITIVE ALLOSTERIC MODULATORS

(71) Applicant: SAGE THERAPEUTICS, INC., Cambridge, MA (US)

(72) Inventors: Michael C. Quirk, Acton, MA (US); Aaron Michael Koenig, Wellesley, MA (US)

(21) Appl. No.: 18/719,194

(22) PCT Filed: Dec. 13, 2022
(86) PCT No.: PCT/US2022/052733

§ 371 Date: Jun. 12, 2024

Related U.S. Application Data
(60) Provisional application No. 63/289,087, filed on Dec. 13, 2021.

(51) Int. Cl. A61K 31/575; A61K 31/4365; A61K 31/4375; A61K 31/4439; A61K 31/454; A61K 31/4545; A61K 31/47; A61K 31/4709; A61K 31/496; A61K 31/5025; A61K 31/517; A61K 31/519; A61K 31/538; A61K 31/58

(52) U.S. Cl. CPC A61K 31/575; A61K 31/4365; A61K 31/4375; A61K 31/4439; A61K 31/454; A61K 31/4545; A61K 31/47; A61K 31/4709; A61K 31/496; A61K 31/5025; A61K 31/517; A61K 31/519; A61K 31/538; A61K 31/58

(57) ABSTRACT

The present disclosure relates to compositions, kits, and methods of treating CNS diseases and disorders using a combination of a muscarinic receptor positive modulator and an NMDA receptor positive allosteric modulator.

This application claims priority to and the benefit of U.S. Provisional Application No. 63/289,087, filed Dec. 13, 2021. The disclosure of the foregoing application is incorporated by reference herein in its entirety.

### BACKGROUND

Muscarinic receptors (mAChRs) belong to the family of plasma membrane-bound G protein coupled receptors (GPCRs) that are widely expressed in the CNS where they control a variety of neuronal functions. The muscarinic component of the central cholinergic system has been implicated in the pathophysiology of numerous disorders, both psychiatric and neurological in nature and have long been a target of the drug discovery industry.

NMDA receptors are heteromeric complexes comprised of NR1, NR2, and/or NR3 subunits and possess distinct recognition sites for exogenous and endogenous ligands. These recognition sites include binding sites for glycine, and glutamate agonists and modulators. NMDA receptors are expressed in the peripheral tissues and the CNS, where they are involved in excitatory synaptic transmission. Activating these receptors contributes to synaptic plasticity in some circumstances and excitotoxicity in others. These receptors are ligand-gated ion channels that admit Ca²⁺ after binding of the glutamate and glycine and are fundamental to excitatory neurotransmission and normal CNS function. Positive modulators (PAM) may be useful as therapeutic agents with potential clinical uses as cognitive enhancers and in the treatment of psychiatric disorders in which glutamatergic transmission is reduced or defective (see, e.g., Horak et al., J. of Neuroscience, 2004, 24 (46), 10318-10325).

Therefore, there remains a need for new and improved therapeutics for treating these and other CNS-related diseases and disorders. The compositions, kits and methods disclosed herein are directed toward this end.

### SUMMARY

In one aspect, the disclosure provides a pharmaceutical composition comprising: (a) a muscarinic receptor positive modulator; and (b) an NMDA receptor positive allosteric modulator.

In one aspect, the disclosure provides a kit comprising a first container, a second container and a package insert, wherein: the first container comprises a composition comprising a muscarinic receptor positive modulator; the second container comprises a composition comprising an NMDA receptor positive allosteric modulator; and the package insert comprises instructions for treating a CNS disease or disorder in a subject.

In one aspect, the disclosure provides a method for treating a disorder described herein, comprising administering to a subject in need thereof an effective amount of a muscarinic receptor positive modulator and an NMDA receptor positive allosteric modulator.

In one aspect, the disclosure provides a method for preventing a disorder described herein, comprising administering to a subject in need thereof an effective amount of a muscarinic receptor positive modulator and an NMDA receptor positive allosteric modulator.

In one aspect, the disclosure provides a pharmaceutical composition as disclosed herein for use in treating a disorder described herein.

In one aspect, the disclosure provides a pharmaceutical composition as disclosed herein for use in preventing a disorder described herein.

In one aspect, the disclosure provides the use of a pharmaceutical composition as disclosed herein for the manufacture of a medicament for treating a disorder described herein.

In one aspect, the disclosure provides the use of a pharmaceutical composition as disclosed herein for the manufacture of a medicament for preventing a disorder described herein.

In some embodiments, the muscarinic receptor positive modulator is a muscarinic receptor agonist.

In some embodiments, the muscarinic receptor agonist is an M1 muscarinic receptor agonist. In some embodiments, the M1 muscarinic receptor agonist is selected from the group consisting of Xanomeline, Cevimeline, AF267B, HTL-9936, AC-260584, AE51090, compounds A1-A5, 77-LH-28-1, EUK1001, RS-86, Talsaclidine, LY-287041 and pharmaceutically acceptable salts thereof.

In some embodiments, the muscarinic receptor agonist is an M2 muscarinic receptor agonist. In some embodiments, the M2 muscarinic receptor agonist is selected from the group consisting of Berberine, Arecaidine Propargyl Ester (APE), Oxotremorine, iperoxo and pharmaceutically acceptable salts thereof.

In some embodiments, the muscarinic receptor agonist is an M3 muscarinic receptor agonist. In some embodiments, the M3 muscarinic receptor agonist, is selected from the group consisting of acetylcholine, bethanechol, carbachol, L-689660, oxotremorine, pilocarpine, muscarine and pharmaceutically acceptable salts thereof.

In some embodiments, the muscarinic receptor agonist is an M4 muscarinic receptor agonist. In some embodiments, the M4 muscarinic receptor agonist is selected from the group consisting of acetylcholine, carbachol. Oxotremorine, Xanomeline, clozapine, EUK1001, and pharmaceutically acceptable salts thereof.

In some embodiments, the muscarinic receptor modulator is an M5 muscarinic receptor agonist. In some embodiments, the M5 muscarinic receptor agonist, is selected from the group consisting of Milameline, Sabcomeline, and pharmaceutically acceptable salts thereof.

In some embodiments, the muscarinic receptor positive modulator is selected from the group consisting Methacholine, Xanomeline, Cevimeline, AF267B, HTL-9936, AC-260584, AE51090, compounds A1-A5, 77-LH-28-1, Berberine, Arecaidine Propargyl Ester (APE), Oxotremorine, iperoxo, acetylcholine, bethanechol, carbachol, L-689660, Pilocarpine, muscarine, Xanomeline, clozapine, Milameline, Sabcomeline, LY-287041, Talsaclidine, EUK1001, RS-86 and pharmaceutically acceptable salts thereof.

In some embodiments, the muscarinic receptor positive modulator is a muscarinic receptor positive allosteric modulator.

In some embodiments, the muscarinic receptor positive allosteric modulator is an M1 muscarinic receptor positive allosteric modulator. In some embodiments, the M1 muscarinic receptor positive allosteric modulator is selected from the group consisting of GSK-1034702, TBPB, PQCA, BQCA, BQZ12, MIPS1674, VU0029767, VU0090157, VU0027414, VU0119498, VU0366369, VU0405562, VU0486846, VU0453595, VU0405652, and VU0405645, N-desmethylclozapine, 77-LH-28-1, AC-42, Brucine, ASN-408, TAK-071, 1-(3-[4-butylpiperidino]propyl)-1,3-dihydrobenzimidazol-2-one, compounds A6-A51, MK-7622, and pharmaceutically acceptable salts thereof.

In some embodiments, the muscarinic receptor positive allosteric modulator is an M2 muscarinic receptor positive allosteric modulator. In some embodiments, the M2 muscarinic receptor positive allosteric modulator is selected from the group consisting of LY2119620, and pharmaceutically acceptable salts thereof.

In some embodiments, the muscarinic receptor positive allosteric modulator is an M3 muscarinic receptor positive allosteric modulator. In some embodiments, the M3 muscarinic receptor allosteric modulator is selected from the group consisting of Amiodarone, N-Ethylamiodarone, and pharmaceutically acceptable salts thereof.

In some embodiments, the muscarinic receptor positive allosteric modulator is an M4 muscarinic receptor positive allosteric modulator. In some embodiments, the M4 muscarinic receptor positive allosteric modulator is selected from the group consisting of LY2033298, LY2119620, VU-0152100, VU00152099, VU0467485, VU10010, and pharmaceutically acceptable salts thereof.

In some embodiments, the muscarinic receptor positive allosteric modulator is an M5 muscarinic receptor allosteric modulator. In some embodiments, the M5 muscarinic receptor positive allosteric modulator is selected from the group consisting of ML-380, ML-326, VU00238429, VU0365114, and pharmaceutically acceptable salts thereof.

In some embodiments, the muscarinic receptor positive modulator is selected from the group consisting of, GSK-1034702, TBPB, PQCA, BQCA, BQZ12, MIPS1674, VU0029767, VU0090157, VU0027414, VU0119498, VU0366369, VU0405562, VU0486846, VU0453595, VU0405652, and VU0405645, N-desmethylclozapine, 77-LH-28-1, AC-42, Brucine, ASN-408, TAK-071, 1-(3-[4-butylpiperidino]propyl)-1,3-dihydrobenzimidazol-2-one, compounds A6-A51, MK-7622, LY2119620, Amiodarone, N-Ethylamiodarone, LY2033298, VU-0152100, VU00152099, VU0467485, VU10010, ML-380, ML-326, VU00238429, VU0365114, and pharmaceutically acceptable salts thereof.

In some embodiments, the muscarinic receptor positive modulator is a muscarinic receptor partial agonist. In some embodiments, the muscarinic receptor partial agonist is an M1 muscarinic receptor partial agonist. In some embodiments, the muscarinic receptor partial agonist is an M2 muscarinic receptor partial agonist. In some embodiments, the muscarinic receptor partial agonist is an M3 muscarinic receptor partial agonist. In some embodiments, the muscarinic receptor partial agonist is an M4 muscarinic receptor partial agonist. In some embodiments, the muscarinic receptor partial agonist is an M5 muscarinic receptor partial agonist. In some embodiments, the muscarinic receptor partial agonist is selected from the group consisting of AF150 (S), SB 202026, (2S,2′R,3'S,5′R)-1-methyl-2-(2-methyl-1,3-oxathiolan-5-yl) pyrrolidine 3-sulfoxide methyl iodide, Iper-8-Naph (N-8-Iper), oxotremorine, Talsaclidine and pilocarpine and pharmaceutically acceptable salts thereof.

In some embodiments, the muscarinic receptor positive modulator is, selected from the group consisting of Methacholine, Xanomeline, Cevimeline, AF267B, HTL-9936, AC-260584, AE51090, compounds A1-A5, 77-LH-28-1, Berberine, Arecaidine Propargyl Ester (APE), Oxotremorine, iperoxo, acetylcholine, bethanechol, carbachol, L-689660, Pilocarpine, muscarine, LY-287041, RS-86, EUK1001, clozapine, Milameline, Sabcomeline, GSK-1034702, TBPB, PQCA, BQCA, BQZ12, MIPS1674, VU0029767, VU0090157, VU0027414, VU0119498, VU0366369, VU0405562, VU0486846, VU0453595, VU0405652, and VU0405645, N-desmethylclozapine, 77-LH-28-1, AC-42, Brucine, ASN-408, TAK-071, 1-(3-[4-butylpiperidino]propyl)-1,3-dihydrobenzimidazol-2-one, compounds A6-A51, MK-7622, LY2119620, Amiodarone, N-Ethylamiodarone, LY2033298, VU-0152100, VU00152099, VU0467485, VU10010, ML-380, ML-326, VU00238429, VU0365114, AF150 (S), SB 202026, (2S,2′R,3'S,5′R)-1-methyl-2-(2-methyl-1,3-oxathiolan-5-yl) pyrrolidine 3-sulfoxide methyl iodide, Iper-8-Naph (N-8-Iper), oxotremorine, Talsaclidine, and pilocarpine and pharmaceutically acceptable salts thereof.

In some embodiments, the NMDA receptor positive allosteric modulator is selected from the group consisting of phenanthrene carboxylic acids, naphthoic acids, benzenesulfonamides, thiazoles, pyrrolidinones, tetrahydroisoquinolines, neuroactive steroids, and pharmaceutically acceptable salts thereof.

In some embodiments, the NMDA receptor positive allosteric modulator is a phenanthrene or a pharmaceutically acceptable salt thereof. In some embodiments, the phenanthrene is UBP512, and pharmaceutically acceptable salts thereof.

In some embodiments, the NMDA receptor positive modulator is a naphthoic acid or a pharmaceutically acceptable salt thereof. In some embodiments, the naphthoic acid is UBP684, and pharmaceutically acceptable salts thereof.

In some embodiments, the NMDA receptor positive modulator is a benzenesulfonamide or a pharmaceutically acceptable salt thereof. In some embodiments, the benzenesulfonamide is GNE-9278, and pharmaceutically acceptable salts thereof.

In some embodiments, the NMDA receptor positive modulator is a thiazole or a pharmaceutically acceptable salt thereof. In some embodiments, the thiazole is GNE-4376, and pharmaceutically acceptable salts thereof.

In some embodiments, the NMDA receptor positive allosteric modulator is a pyrrolidinone or a pharmaceutically acceptable salt thereof. In some embodiments, the pyrrolidinone is selected from a group consisting of PYD-111 and PYD-106, and pharmaceutically acceptable salts thereof.

In some embodiments, the NMDA receptor positive allosteric modulator is a tetrahydroisoquinoline, and pharmaceutically acceptable salts thereof. In some embodiments, the tetrahydroisoquinoline is CIQ, and pharmaceutically acceptable salts thereof.

In some embodiments, the NMDA receptor positive allosteric modulator is a neuroactive steroid or a pharmaceutically acceptable salt thereof. In some embodiments, the neuroactive steroid is selected from any one of the compounds B1-B659, and pharmaceutically acceptable salts thereof. In some embodiments, the neuroactive steroid is selected from Compounds B164, B351, B411, and pharmaceutically acceptable salts thereof. In some embodiments, the neuroactive steroid is B164, or a pharmaceutically acceptable salt thereof. In some embodiments, the neuroactive steroid is B351, or a pharmaceutically acceptable salt thereof. In some embodiments, the neuroactive steroid is B411, or a pharmaceutically acceptable salt thereof.

In some embodiments, the disorder is a gastrointestinal (GI) disorder e.g., constipation, irritable bowel syndrome (IBS), inflammatory bowel disease (IBD) (e.g., ulcerative colitis, Crohn's disease), structural disorders affecting the GI, anal disorders (e.g., hemorrhoids, internal hemorrhoids, external hemorrhoids, anal fissures, perianal abscesses, anal fistula), colon polyps, cancer, colitis.

In some embodiments, the disorder is inflammatory bowel disease.

In some embodiments, the disorder is cancer, diabetes, or a sterol synthesis disorder.

In some embodiments, the disorder is a sterol synthesis disorder.

In some embodiments, the disorder is a CNS-related condition. In some embodiments, the CNS-related condition is an adjustment disorder, anxiety disorder (including obsessive-compulsive disorder, posttraumatic stress disorder, and social phobia), cognitive disorder (including Alzheimer's disease, cancer-related cognitive impairment, treatment of residual cognitive symptoms, and other forms of dementia (e.g., frontotemporal dementia)), delirium, dissociative disorder, eating disorder, mood disorder (including depression (e.g., postpartum depression), bipolar disorder, dysthymic disorder, major depressive disorder, suicidality), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins (e.g., Shank3)), neurodevelopmental disorder (including Rett syndrome, Tuberous Sclerosis complex), multiple sclerosis, pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease), vision impairment, hearing loss, and tinnitus.

In some embodiments, the muscarinic receptor positive modulator and the NMDA receptor positive allosteric modulator are administered simultaneously.

In some embodiments, the muscarinic receptor positive modulator and the NMDA receptor positive allosteric modulator are administered sequentially. In some embodiments, the muscarinic receptor positive modulator and the NMDA receptor positive allosteric modulator are administered separately. In some embodiments, the muscarinic receptor positive modulator is administered before the NMDA receptor positive allosteric modulator. In some embodiments, the NMDA receptor positive allosteric modulator is administered before the muscarinic receptor positive modulator. In some embodiments, the frequency of administration of the muscarinic receptor positive modulator and the NMDA receptor positive allosteric modulator is the same. In some embodiments, the frequency of administration of the muscarinic receptor positive modulator and the NMDA receptor positive allosteric modulator is different.

### DETAILED DESCRIPTION

As generally described herein, the present invention provides compositions, kits and methods for treating various CNS diseases and disorders in a subject in need thereof.

### Definitions

The term “herein” means the entire application.

Unless otherwise defined herein, scientific and technical terms used in this application shall have the meanings that are commonly understood by those of ordinary skill in the art to which this invention belongs. Generally, nomenclature used in connection with the compounds, composition and methods described herein, are those well-known and commonly used in the art.

It should be understood that any of the embodiments described herein, including those described under different aspects of the disclosure and different parts of the specification (including embodiments described only in the Examples) can be combined with any one or more other embodiments of the invention, unless explicitly disclaimed or improper. Combination of embodiments are not limited to those specific combinations claimed via the multiple dependent claims. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein.

All of the publications, patents and published patent applications referred to in this application are specifically incorporated by reference herein in their entirety. In case of conflict, the present specification, including its specific definitions, will control.

Definitions of specific functional groups and chemical terms are described in more detail below. The chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5th Edition, John Wiley & Sons, Inc., New York, 2001; Larock, Comprehensive Organic Transformations, VCH Publishers, Inc., New York, 1989; and Carruthers, Some Modern Methods of Organic Synthesis, 3rd Edition, Cambridge University Press, Cambridge, 1987.

Throughout this specification, the word “comprise” or variations such as “comprises” or “comprising” will be understood to imply the inclusion of a stated integer (element or component) or group of integers (elements or components), but not the exclusion of any other integer (element or component) or group of integers (elements or components).

Throughout the specification, where compositions are described as having, including, or comprising (or variations thereof), specific components, it is contemplated that compositions also may consist essentially of, or consist of, the recited components. Similarly, where methods or processes are described as having, including, or comprising specific process steps, the processes also may consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

The term “including,” as used herein, means “including but not limited to.” “Including” and “including but not limited to” are used interchangeably. Thus, these terms will be understood to imply the inclusion of a stated integer (element or component) or group of integers (elements or components), but not the exclusion of any other integer (element or component) or group of integers (elements or components).

As used herein, “about” or “approximately” means within an acceptable error range for the particular value as determined by one of ordinary skill in the art, which will depend in part on how the value is measured or determined, i.e., the limitations of the measurement system.

Unless otherwise required by context, singular terms shall include pluralities and plural terms shall include the singular.

The use of the terms “a” and “an” and “the” and similar referents in the context of describing the elements (especially in the context of the following claims) are to be construed to cover both the singular and the plural, unless otherwise indicated herein or clearly contradicted by context.

The term “or” as used herein should be understood to mean “and/or,” unless the context clearly indicates otherwise.

Recitation of ranges of values herein are merely intended to serve as a shorthand method of referring individually to each separate value falling within the range, unless otherwise indicated herein, and each separate value is incorporated into the specification as if it were individually recited herein. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10. The disclosure of a range should also be considered as disclosure of the endpoints of that range.

All methods described herein can be performed in any suitable order unless otherwise indicated herein or otherwise clearly contradicted by context.

The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, the term “positive modulation” or variations thereof as it relates to receptors (e.g., NMDA receptor or muscarinic receptor) refers to the potentiation of or increase in receptor activity and/or function. A “positive modulator” (e.g., a modulator compound) may be, for example, an agonist, a partial agonist, a positive allosteric modulator and an allosteric agonist.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. The term “pharmaceutically acceptable cation” refers to an acceptable cationic counter-ion of an acidic functional group. Such cations are exemplified by sodium, potassium, calcium, magnesium, ammonium, tetraalkylammonium cations, and the like. See, e.g., Berge, et al., J. Pharm. Sci. (1977) 66 (1): 1-79.

“Stereoisomers”: It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

“Tautomers” refer to compounds that are interchangeable forms of a particular compound structure, and that vary in the displacement of hydrogen atoms and electrons. Thus, two structures may be in equilibrium through the movement of x electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a compound of interest.

As used herein, the term “unit dosage form” is defined to refer to the form in which a compound as disclosed herein is administered to the subject.

As used herein, the term “daily” means a given, continuous twenty-four (24) hour period.

The terms “subject,” “patient” and “individual” are used interchangeably herein and include, but are not limited to, humans and a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human (“human subject”). In certain embodiments, the human subject is an infant, child, or adolescent (“pediatric subject”). In other embodiments, the human subject is a young adult, middle-aged adult or senior adult (“adult subject”). In certain embodiments, the subject is a non-human animal (“non-human subject”).

Disease, disorder, and condition are used interchangeably herein.

As used herein, and unless otherwise specified, the terms “treat,” “treating” and “treatment” contemplate an action that occurs while a subject is suffering from the specified disease, disorder, or condition, which reduces the severity of the disease, disorder, or condition, or retards or slows the progression of the disease, disorder, or condition (also, “therapeutic treatment”). As used herein, and as well understood in the art, “treatment” is an approach for obtaining beneficial or desired results, including clinical results. Beneficial or desired clinical results can include, but are not limited to, alleviation, amelioration, reduction of the severity, or slowing the progression, of one or more symptoms or conditions associated with a condition, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Exemplary beneficial clinical results are described herein.

In general, the “effective amount” of a compound or pharmaceutical composition refers to an amount sufficient to elicit the desired biological response. As will be appreciated by those of ordinary skill in this art, the effective amount of a compound or pharmaceutical composition of the invention may vary depending on such factors as the desired biological endpoint, the pharmacokinetics of the compound, the disease being treated, the mode of administration, and the age, weight, health, and condition of the subject.

As used herein, “therapeutically effective amount” of a compound or pharmaceutical composition is an amount sufficient to provide a therapeutic benefit in the treatment of a disease, disorder or condition, to delay or minimize one or more symptoms associated with the disease, disorder or condition, or to improve cognitive function. A therapeutically effective amount of a compound or pharmaceutical composition means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, the nature and extent of disease, the therapeutics or combination of therapeutics selected for administration, and the mode of administration. The skilled worker can readily determine the effective amount for a given situation by routine experimentation. The terms “therapeutically effective amount,” or “therapeutically effective dose” also refer to the amount required to improve the clinical symptoms of a patient. A therapeutically effective amount of a compound or pharmaceutical composition also refers to an amount of the compound or pharmaceutical compositions, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

In an alternative embodiment, the compounds, pharmaceutically acceptable salts thereof or pharmaceutically acceptable composition thereof, can also be administered in a “prophylactically effective amount”. As used herein, and unless otherwise specified, a “prophylactically effective amount” of a compound or pharmaceutical composition is an amount sufficient to prevent a disease, disorder or condition, or one or more symptoms associated with the disease, disorder or condition, or prevent its recurrence. A prophylactically effective amount of a compound or pharmaceutical composition means an amount of a therapeutic agent or pharmaceutical composition, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

“Administering” or “administration of” a substance, a compound, agent or pharmaceutical composition to a subject can be carried out using one of a variety of methods known to those skilled in the art. For example, a compound, agent or pharmaceutical composition can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound, agent or pharmaceutical composition can also appropriately be introduced by rechargeable or biodegradable polymeric devices or other devices, e.g., patches and pumps, or formulations, which provide for the extended, slow or controlled release of the compound, agent or pharmaceutical composition. Administering can also be performed, for example, once, a plurality of times, and/or over one or more extended periods. In some embodiments, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient. When a method is part of a therapeutic regimen involving more than one agent or treatment modality, the disclosure contemplates that the agents may be administered at the same or differing times and via the same or differing routes of administration. Appropriate methods of administering a substance, a compound, an agent or pharmaceutical composition to a subject will also depend, for example, on the age of the subject, whether the subject is active or inactive at the time of administering, whether the subject is cognitively impaired at the time of administering, the extent of the impairment, and the chemical and biological properties of the compound, agent or pharmaceutical composition (e.g. solubility, digestibility, bioavailability, stability and toxicity).

### General

Disclosed herein are pharmaceutical compositions, kits and methods of treatment comprising a muscarinic receptor positive modulator and an NMDA Receptor Positive Allosteric Modulator (NMDA PAM).

### Muscarinic Receptor Positive Modulator

The muscarinic receptor positive modulator useful in the pharmaceutical compositions, kits and methods of the disclosure is a compound that potentiates or increases the activity or function of at least one muscarinic receptor. Examples of muscarinic receptor positive modulators include muscarinic receptor agonists, muscarinic receptor partial agonists, muscarinic receptor positive allosteric modulators (including allosteric agonists) and/or indirect-acting (reversible or irreversible) cholinergic agents (i.e., agents that increase the availability of acetylcholine at the cholinergic receptors, e.g, Acetyl Choline Esterase Inhibitors (AChEIs)). Muscarinic receptor positive modulators are known to those skilled in the art. Therefore, any muscarinic receptor positive modulator may be useful in the compositions, kits and methods of the present disclosure.

In some embodiments, the muscarinic receptor positive modulator is selected from a muscarinic receptor agonist, muscarinic receptor partial agonists, muscarinic receptor positive allosteric modulators and/or AChEIs.

In some embodiments, the muscarinic receptor positive modulator is a muscarinic receptor agonist. In some embodiments, the muscarinic receptor agonist is an agonist of at least one subtype of a muscarinic receptor (e.g., M1, M2, M3, M4 or M5).

In some embodiments, the muscarinic receptor agonist is an M1 muscarinic receptor agonist. Exemplary M1 muscarinic receptor agonists include, but are not limited to, Xanomeline, Cevimeline, AF267B, HTL-9936, AC-260584, AE51090, compounds A1-A5, 77-LH-28-1, EUK1001, RS-86, Talsaclidine, LY-287041 and pharmaceutically acceptable salts thereof.

In some embodiments, the muscarinic receptor agonist is an M2 muscarinic receptor agonist. Exemplary M2 muscarinic receptor agonists include, but are not limited to, Berberine, Arecaidine Propargyl Ester (APE), Oxotremorine, iperoxo and pharmaceutically acceptable salts thereof.

In some embodiments, the muscarinic receptor agonist is an M3 muscarinic receptor agonist. Exemplary M3 muscarinic receptor agonists include, but are not limited to, acetylcholine, bethanechol, carbachol, L-689660, oxotremorine, pilocarpine, muscarine and pharmaceutically acceptable salts thereof.

In some embodiments, the muscarinic receptor agonist is an M4 muscarinic receptor agonist. Exemplary M4 muscarinic receptor agonists include, but are not limited to, acetylcholine, carbachol, Oxotremorine, Xanomeline, clozapine, EUK1001, and pharmaceutically acceptable salts thereof.

In some embodiments, the muscarinic receptor modulator is an M5 muscarinic receptor agonist. Exemplary M5 muscarinic receptor agonists include, but are not limited to, Milameline and Sabcomeline, and pharmaceutically acceptable salts thereof.

In some embodiments, the muscarinic receptor positive modulator includes, but is not limited to Methacholine, Xanomeline, Cevimeline, AF267B, HTL-9936, AC-260584, AE51090, compounds A1-A5, 77-LH-28-1, Berberine, Arecaidine Propargyl Ester (APE), Oxotremorine, iperoxo, acetylcholine, bethanechol, carbachol, L-689660, Pilocarpine, muscarine, clozapine, Milameline, Sabcomeline, LY-287041, Talsaclidine, EUK1001, RS-86.

In some embodiments, the muscarinic receptor positive modulator is a muscarinic receptor positive allosteric modulator. In some embodiments, the muscarinic receptor positive allosteric modulator is a muscarinic receptor allosteric agonist.

In some embodiments, the muscarinic receptor positive allosteric modulator is an M1 muscarinic receptor positive allosteric modulator. Exemplary M1 muscarinic receptor positive allosteric modulators include, but are not limited to, GSK-1034702, TBPB, PQCA, BQCA, BQZ12, MIPS1674, VU0029767, VU0090157, VU0027414, VU0119498, VU0366369, VU0405562, VU0486846, VU0453595, VU0405652, and VU0405645, N-desmethylclozapine, 77-LH-28-1, AC-42, Brucine, ASN-408, TAK-071, 1-(3-[4-butylpiperidino]propyl)-1,3-dihydrobenzimidazol-2-one, compounds A6-A51, MK-7622, and pharmaceutically acceptable salts thereof.

In some embodiments, the muscarinic receptor positive allosteric modulator is an M2 muscarinic receptor positive allosteric modulator. ExemplaryM2 muscarinic receptor positive allosteric modulators include, but are not limited to, LY2119620, and pharmaceutically acceptable salts thereof.

In some embodiments, the muscarinic receptor positive allosteric modulator is an M3 muscarinic receptor positive allosteric modulator. Exemplary M3 muscarinic receptor allosteric modulators include, but are not limited to, Amiodarone, N-Ethylamiodarone, and pharmaceutically acceptable salts thereof.

In some embodiments, the muscarinic receptor positive allosteric modulator is an M4 muscarinic receptor positive allosteric modulator. Exemplary M4 muscarinic receptor positive allosteric modulators include, but are not limited to, LY2033298, LY2119620, NMRA-266, VU-0152100, VU00152099, VU0467154 (5-amino-3,4-dimethyl-N-(4-((trifluoromethyl) sulfonyl) benzyl) thieno[2,3-c]pyridazine-6-carboxamide), VU0467485, VU0476406, VU10010, VU0409524, VU0476319, VU6002703, VU6009453, VU0619856, VU6009003, and pharmaceutically acceptable salts thereof. In some embodiments, the M4 muscarinic receptor positive allosteric modulator includes, but is not limited to, LY2033298, LY2119620, VU-0152100, VU00152099, VU0467485, VU10010, and pharmaceutically acceptable salts thereof. In some embodiments, the muscarinic receptor positive modulator is a M4 positive allosteric modulator, wherein the M4 positive allosteric modulator is NMRA-266.

Additional exemplary M4 muscarinic receptor positive allosteric modulator include, but are not limited to: 1)N-(3-fluoro-4-methoxybenzyl)-3,4-dimethylpyrimido[4′,5′: 4,5]thieno[2,3-c]pyridazin-8-amine; 2) 2-[4-[[(3,4-Dimethylpyrimido[4′,5′: 4,5]thieno[2,3-c]pyridazin-8-yl) amino]methyl]phenyl]propan-2-ol; 3)N-[1-(5-fluoropyrimidin-2-yl) azetidin-3-yl]-3,4-dimethyl-pyrimido[4′,5′: 4,5]thieno[2,3-c]pyridazin-8-amine; 4) 2-[4-[1-[(3,4-dimethylpyrimido[4,5′: 4,5]thieno[2,3-c]pyridazin-8-yl) amino]ethyl]phenyl]propan-2-ol; 5) 2-[5-[(3,4-dimethylpyrimido[4′,5′: 4,5]thieno[2,3-c]pyridazin-8-yl) amino]methyl]-2-pyridyl]propan-2-ol; 6) 2-[1-[[4-[[(3,4-dimethylpyrimido[4′,5′: 4,5]thieno[2,3-c]pyridazin-8-yl) amino]methyl]phenyl]methyl]pyrrolidin-2-yl]propan-2-ol; 7)N-(3,3-difluorocyclobutyl)-4-[(3,4-dimethylpyrimido[4′,5′: 4,5]thieno[2,3-c]pyridazin-8-yl) amino]methyl]benzamide; 8) 3-chloro-N-cyclobutyl-4-methyl-pyrimido[4′,5′: 4,5]thieno[2,3-c]pyridazin-8-amine; 9) 2-[4-[[(3-chloro-4-methyl-pyrimido[4′,5′: 4,5]thieno[2,3-c]pyridazin-8-yl) amino]methyl]phenyl]propan-2-ol; 10) 2-[4-[dideuterio-[(3,4-dimethylpyrimido[4′,5′: 4,5]thieno[2,3-c]pyridazin-8-yl) amino]methyl]phenyl]propan-2-ol; 11)N-[(1,1-dimethyl-3H-isobenzofuran-5-yl)methyl]-3,4-dimethyl-pyrimido[4,5′: 4,5]thieno[2,3-c]pyridazin-8-amine; 12) 2-[4-[[(3,4-dimethylpyrimido[4′,5′: 4,5]thieno[2,3-c]pyridazin-8-yl) amino]methyl]phenyl]propane-1,2-diol; 13) 2-[4-[[(3,4-dimethylpyrimido[4′,5′: 4,5]thieno[2,3-c]pyridazin-8-yl) amino]methyl]phenyl]-2-methyl-propan-1-ol; 14) (8-amino-9-methylthieno[3,2-e][1,2,4]triazolo[4,3-b]pyridazin-7-yl) (3-phenylpyrrolidin-1-yl) methanone; 15) (8-amino-9-methylthieno[3,2-e][1,2,4]triazolo[4,3-b]pyridazin-7-yl) (pyrrolidin-1-yl) methanone; 16) 8-amino-N-(2,3-dihydro-1H-inden-2-yl)-9-methylthieno[3,2-e][1,2,4]triazolo[4,3-b]pyridazine-7-carboxamide; 17) 8-amino-N-(4-methoxybenzyl)-9-methylthieno[3,2-e][1,2,4]triazolo[4,3-b]pyridazine-7-carboxamide; 18) 8-amino-N-cyclopropyl-9-methylthieno[3,2-e][1,2,4]triazolo[4,3-b]pyridazine-7-carboxamide; 19) 8-amino-N-cyclobutyl-9-methylthieno[3,2-e][1,2,4]triazolo[4,3-b]pyridazine-7-carboxamide; 20) 8-amino-9-methyl-N-(1-phenylpiperidin-4-yl) thieno[3,2-e][1,2,4]triazolo[4,3-b]pyridazine-7-carboxamide; 21) 8-amino-N-(3-fluorobenzyl)-9-methylthieno[3,2-e][1,2,4]triazolo[4,3-b]pyridazine-7-carboxamide; 22) 8-amino-N-(4-fluorobenzyl)-9-methylthieno[3,2-e][1,2,4]triazolo[4,3-b]pyridazine-7-carboxamide; and 23) 5-amino-3-methoxy-4-methyl-N-(1-(5-(trifluoromethyl) pyrimidin-2-yl) azetidin-3-yl) thieno[2,3-c]pyridazine-6-carboxamide; or a pharmaceutically acceptable salt thereof.

Exemplary muscarinic receptor positive allosteric modulators are disclosed in WO2012154731, WO2013040534, WO2013126856, WO2014035829, WO2015027204, WO2015027214, WO2017223290, WO2018035444, WO2018085803, WO2018085808, WO2018085813, WO2018112312, WO2019036534, WO2019113174, WO2022015988, WO2022221556, US20140364409, US20180086776, US20180028501, U.S. Pat. Nos. 10,239,887, 10,189,857, the disclosures of each of which are incorporated by reference herein in their entirety.

In some embodiments, the muscarinic receptor positive allosteric modulator is an M5 muscarinic receptor allosteric modulator. Exemplary M5 muscarinic receptor positive allosteric modulators include, but are not limited to ML-380, ML-326, VU00238429, VU0365114, and pharmaceutically acceptable salts thereof.

In some embodiments, the muscarinic receptor positive modulator includes, but is not limited to, GSK-1034702, TBPB, PQCA, BQCA, BQZ12, MIPS1674, VU0029767, VU0090157, VU0027414, VU0119498, VU0366369, VU0405562, VU0486846, VU0453595, VU0405652, and VU0405645, N-desmethylclozapine, 77-LH-28-1, AC-42, Brucine, ASN-408, TAK-071, 1-(3-[4-butylpiperidino]propyl)-1,3-dihydrobenzimidazol-2-one, compounds A6-A51, MK-7622, LY2119620, Amiodarone, N-Ethylamiodarone, LY2033298, NMRA-266, VU0467154 (5-amino-3,4-dimethyl-N-(4-((trifluoromethyl) sulfonyl) benzyl) thieno[2,3-c]pyridazine-6-carboxamide), VU0476406, VU-0152100, VU00152099, VU0467485, VU10010, VU0409524, VU0476319, VU6002703, VU6009453, VU0619856, VU6009003ML-380, ML-326, VU00238429, VU0365114, and pharmaceutically acceptable salts thereof. In some embodiments, the muscarinic receptor positive modulator includes, but is not limited to GSK-1034702, TBPB, PQCA, BQCA, BQZ12, MIPS1674, VU0029767, VU0090157, VU0027414, VU0119498, VU0366369, VU0405562, VU0486846, VU0453595, VU0405652, and VU0405645, N-desmethylclozapine, 77-LH-28-1, AC-42, Brucine, ASN-408, TAK-071, 1-(3-[4-butylpiperidino]propyl)-1,3-dihydrobenzimidazol-2-one, compounds A6-A51, MK-7622, LY2119620, Amiodarone, N-Ethylamiodarone, LY2033298, VU-0152100, VU00152099, VU0467485, VU10010, ML-380, ML-326, VU00238429, VU0365114 and pharmaceutically acceptable salts thereof.

In some embodiments, the muscarinic receptor positive modulator is a muscarinic receptor partial agonist. In some embodiments, wherein the muscarinic receptor partial agonist is an M1 muscarinic receptor partial agonist. In some embodiments, the muscarinic receptor partial agonist is an M2 muscarinic receptor partial agonist. In some embodiments, the muscarinic receptor partial agonist is an M3 muscarinic receptor partial agonist. In some embodiments, the muscarinic receptor partial agonist is an M4 muscarinic receptor partial agonist. In some embodiments, the muscarinic receptor partial agonist is an M5 muscarinic receptor partial agonist.

Exemplary muscarinic receptor partial agonists include, but are not limited to, AF150 (S), SB 202026, (2S,2′R,3'S,5′R)-1-methyl-2-(2-methyl-1,3-oxathiolan-5-yl) pyrrolidine 3-sulfoxide methyl iodide, Iper-8-Naph (N-8-Iper), oxotremorine, Talsaclidine and pilocarpine and pharmaceutically acceptable salts thereof.

In some embodiments, the muscarinic receptor positive modulator includes, but is not limited to Methacholine, Xanomeline, Cevimeline, AF267B, HTL-9936, AC-260584, AE51090, compounds A1-A5, 77-LH-28-1, Berberine, Arecaidine Propargyl Ester (APE), Oxotremorine, iperoxo, acetylcholine, bethanechol, carbachol, L-689660, Pilocarpine, muscarine, LY-287041, RS-86, EUK1001, Xanomeline, clozapine, Milameline, Sabcomeline, GSK-1034702, TBPB, PQCA, BQCA, BQZ12, MIPS1674, VU0029767, VU0090157, VU0027414, VU0119498, VU0366369, VU0405562, VU0486846, VU0453595, VU0405652, and VU0405645, N-desmethylclozapine, 77-LH-28-1, AC-42, Brucine, ASN-408, TAK-071, 1-(3-[4-butylpiperidino]propyl)-1,3-dihydrobenzimidazol-2-one, compounds A6-A51, MK-7622, LY2119620, Amiodarone, N-Ethylamiodarone, LY2033298, VU-0152100, VU00152099, VU0467485, VU10010, ML-380, ML-326, VU00238429, VU0365114, AF150 (S), SB 202026, (2S,2′R,3'S,5′R)-1-methyl-2-(2-methyl-1,3-oxathiolan-5-yl) pyrrolidine 3-sulfoxide methyl iodide, Iper-8-Naph (N-8-Iper), Talsaclidine, and pharmaceutically acceptable salts thereof.

In some embodiments, the muscarinic receptor positive modulator is an acetylcholinesterase Inhibitor (AChEI). Exemplary AChEIs include, but are not limited to Donepezil, Rivastigmine, memantine, galantamine, tacrine, Physostigmine, Neostigmine, pyridostigmine, Ambenonium, Demecarium, Caffeine, Rosmarinic acid, Alpha-pinene, Edrophonium, Huperzine A, Ladostigil, Ungeremine, Lactucopicrin, Acotiamide, Metrifonate, phenserine, Tolserine, Eseroline, Huperzine, Galangin, and cardanol.

In some embodiments, the muscarinic receptor positive modulator is any compound disclosed in any one of U.S. Pat. Nos. 11,149,036, 11,091,456, 11,040,026, 11,008,335, 10,981,902, 10,961,253, 10,945,771, 10,934,276, 10,927,126, 10,899,759, 10,548,899, 10,351,564, 10,548,884, 10,351,564, 10,329,289, 10,308,620, 10,294,217, 10,239,887, 10,221,175, 10,196,380, 10,167,284, 10,167,272, 9,926,297, 9,868,725, 9,758,506, 9,708,278, 9,670,183, 9,540,371, 9,527,834, 9,199,939, 9,012,445, 8,815,902, 8,697,690, 8,680,115, 8,664,234, 8,557,992, 8,383,638, 8,273,737, 7,576,100, 7,576,078, 7,550,459, 7,307,075, 7,300,928, 7,291,611, 7,087,593, 6,627,645, the disclosures of each of which are incorporated herein by reference in their entirety.

In some embodiments, the muscarinic receptor positive modulator is any one of compounds A1-A51 in Table 1 or a pharmaceutically acceptable salt thereof.

TABLE 1

| No | Structure |
|---|---|
| A1 | 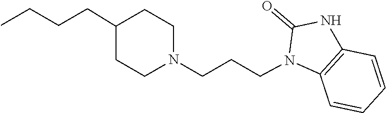 [SMILES: CCCCC1CCN(CCCn2c(=O)[nH]c3ccccc32)CC1] |
| A2 | 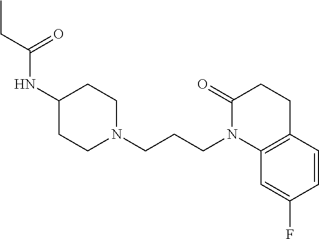 [SMILES: CCC(=O)NC1CCN(CCCN2C(=O)CCc3ccc(F)cc32)CC1] |
| A3 | 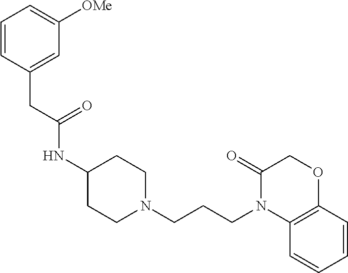 [SMILES: COc1cccc(CC(=O)NC2CCN(CCCN3C(=O)COc4ccccc43)CC2)c1] |
| A4 | 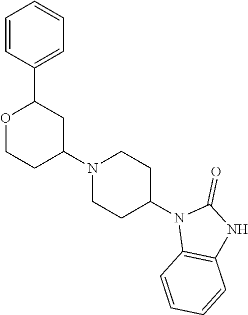 [SMILES: O=c1[nH]c2ccccc2n1C1CCN(C2CCOC(c3ccccc3)C2)CC1] |
| A5 | 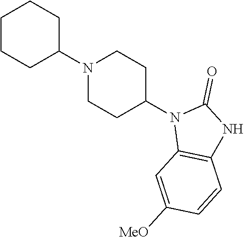 [SMILES: COc1ccc2[nH]c(=O)n(C3CCN(C4CCCCC4)CC3)c2c1] |
| A6 | 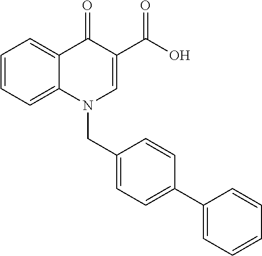 [SMILES: O=C(O)c1cn(Cc2ccc(-c3ccccc3)cc2)c2ccccc2c1=O] |
| A7 | 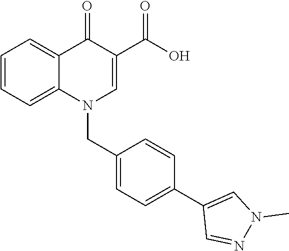 [SMILES: Cn1cc(-c2ccc(Cn3cc(C(=O)O)c(=O)c4ccccc43)cc2)cn1] |
| A8 | 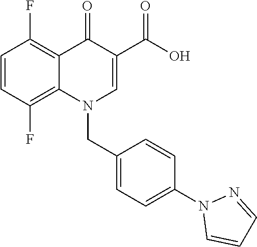 [SMILES: O=C(O)c1cn(Cc2ccc(-n3cccn3)cc2)c2c(F)ccc(F)c2c1=O] |
| A9 | 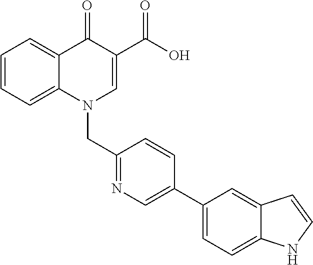 [SMILES: O=C(O)c1cn(Cc2ccc(-c3ccc4[nH]ccc4c3)cn2)c2ccccc2c1=O] |
| A10 | 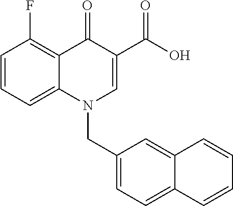 [SMILES: O=C(O)c1cn(Cc2ccc3ccccc3c2)c2cccc(F)c2c1=O] |
| A11 | 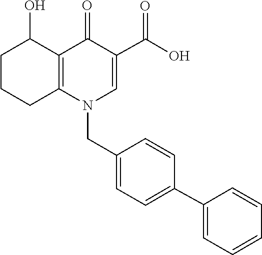 [SMILES: O=C(O)c1cn(Cc2ccc(-c3ccccc3)cc2)c2c(c1=O)C(O)CCC2] |
| A12 | 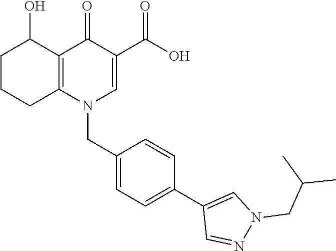 [SMILES: CC(C)Cn1cc(-c2ccc(Cn3cc(C(=O)O)c(=O)c4c3CCCC4O)cc2)cn1] |
| A13 | 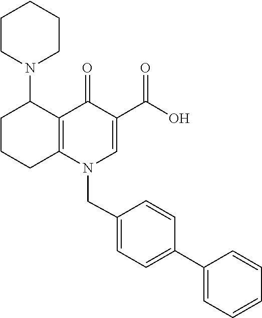 [SMILES: O=C(O)c1cn(Cc2ccc(-c3ccccc3)cc2)c2c(c1=O)C(N1CCCCC1)CCC2] |
| A14 | 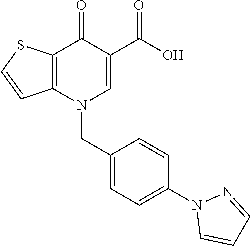 [SMILES: O=C(O)c1cn(Cc2ccc(-n3cccn3)cc2)c2ccsc2c1=O] |
| A15 | 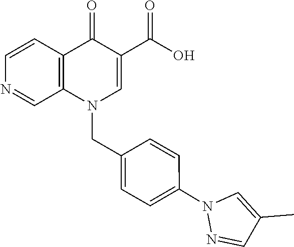 [SMILES: Cc1cnn(-c2ccc(Cn3cc(C(=O)O)c(=O)c4ccncc43)cc2)c1] |
| A16 | 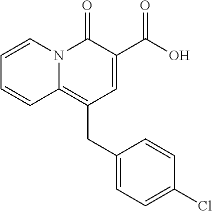 [SMILES: O=C(O)c1cc(Cc2ccc(Cl)cc2)c2ccccn2c1=O] |
| A17 | 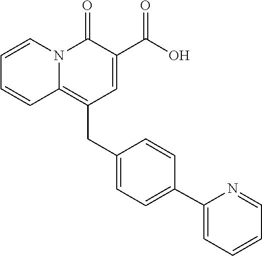 [SMILES: O=C(O)c1cc(Cc2ccc(-c3ccccn3)cc2)c2ccccn2c1=O] |
| A18 | 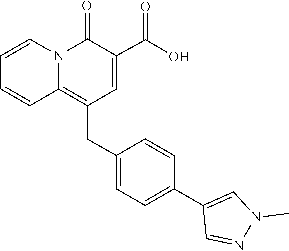 [SMILES: Cn1cc(-c2ccc(Cc3cc(C(=O)O)c(=O)n4ccccc34)cc2)cn1] |
| A19 | 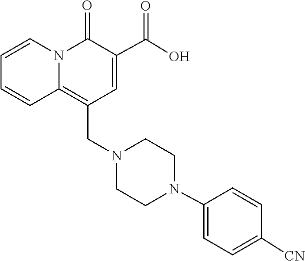 [SMILES: N#Cc1ccc(N2CCN(Cc3cc(C(=O)O)c(=O)n4ccccc34)CC2)cc1] |
| A20 | 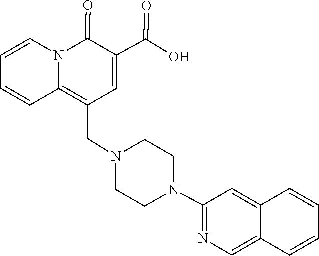 [SMILES: O=C(O)c1cc(CN2CCN(c3cc4ccccc4cn3)CC2)c2ccccn2c1=O] |
| A21 | 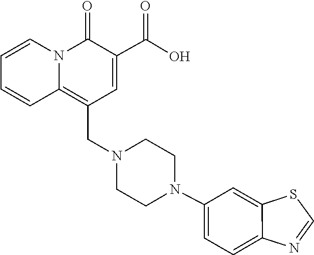 [SMILES: O=C(O)c1cc(CN2CCN(c3ccc4ncsc4c3)CC2)c2ccccn2c1=O] |
| A22 | 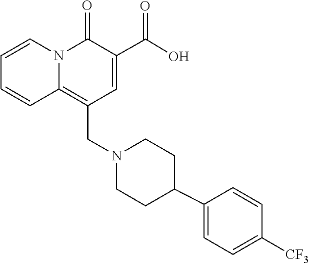 [SMILES: O=C(O)c1cc(CN2CCC(c3ccc(C(F)(F)F)cc3)CC2)c2ccccn2c1=O] |
| A23 | 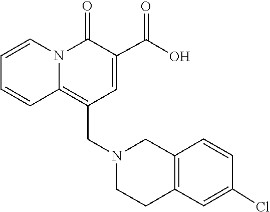 [SMILES: O=C(O)c1cc(CN2CCc3cc(Cl)ccc3C2)c2ccccn2c1=O] |
| A24 | 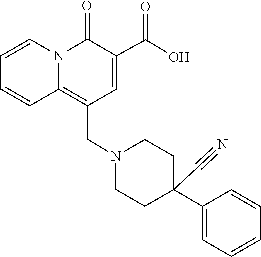 [SMILES: N#CC1(c2ccccc2)CCN(Cc2cc(C(=O)O)c(=O)n3ccccc23)CC1] |
| A25 | 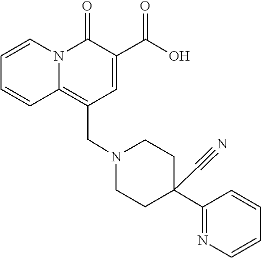 [SMILES: N#CC1(c2ccccn2)CCN(Cc2cc(C(=O)O)c(=O)n3ccccc23)CC1] |
| A26 | 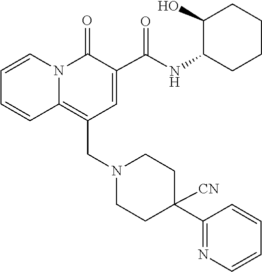 [SMILES: N#CC1(c2ccccn2)CCN(Cc2cc(C(=O)N[C@H]3CCCC[C@@H]3O)c(=O)n3ccccc23)CC1] |
| A27 | 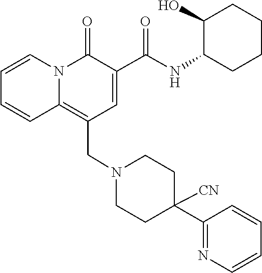 [SMILES: N#CC1(c2ccccn2)CCN(Cc2cc(C(=O)N[C@H]3CCCC[C@@H]3O)c(=O)n3ccccc23)CC1] |
| A28 | 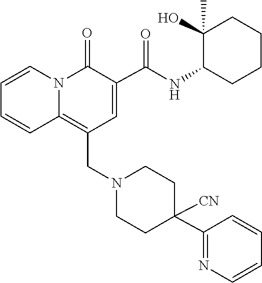 [SMILES: C[C@]1(O)CCCC[C@@H]1NC(=O)c1cc(CN2CCC(C#N)(c3ccccn3)CC2)c2ccccn2c1=O] |
| A29 | 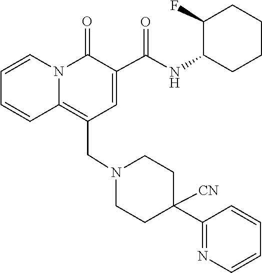 [SMILES: N#CC1(c2ccccn2)CCN(Cc2cc(C(=O)N[C@H]3CCCC[C@@H]3F)c(=O)n3ccccc23)CC1] |
| A30 | 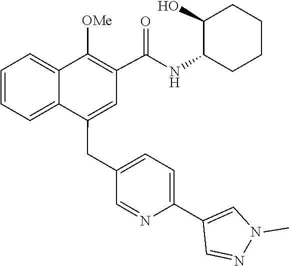 [SMILES: COc1c(C(=O)N[C@H]2CCCC[C@@H]2O)cc(Cc2ccc(-c3cnn(C)c3)nc2)c2ccccc12] |
| A31 | 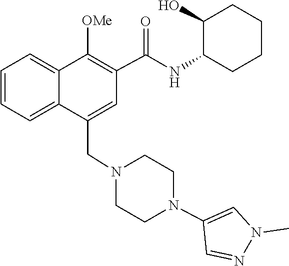 [SMILES: COc1c(C(=O)N[C@H]2CCCC[C@@H]2O)cc(CN2CCN(c3cnn(C)c3)CC2)c2ccccc12] |
| A32 | 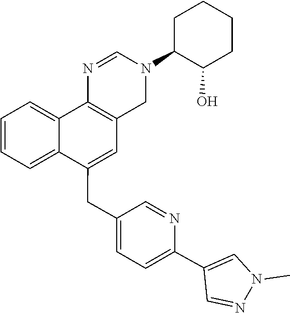 [SMILES: Cn1cc(-c2ccc(Cc3cc4c(c5ccccc35)N=CN([C@H]3CCCC[C@@H]3O)C4)cn2)cn1] |
| A33 | 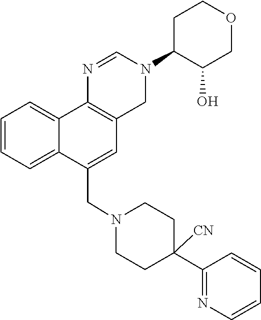 [SMILES: N#CC1(c2ccccn2)CCN(Cc2cc3c(c4ccccc24)N=CN([C@H]2CCOC[C@@H]2O)C3)CC1] |
| A34 | 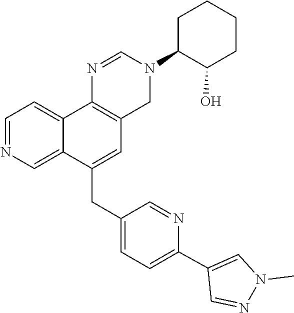 [SMILES: Cn1cc(-c2ccc(Cc3cc4c(c5ccncc35)N=CN([C@H]3CCCC[C@@H]3O)C4)cn2)cn1] |
| A35 | 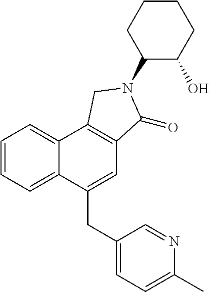 |
| A36 | 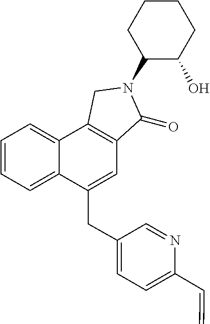 |
| A37 | 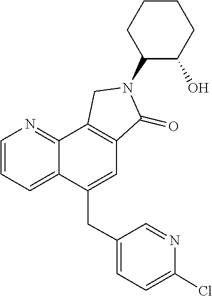 |
| A38 | 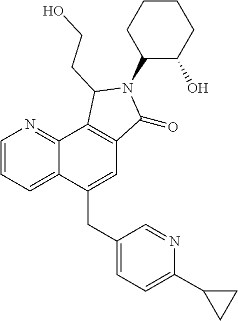 |
| A39 | 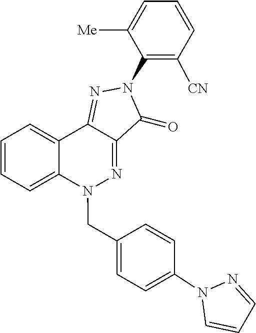 |
| A40 | 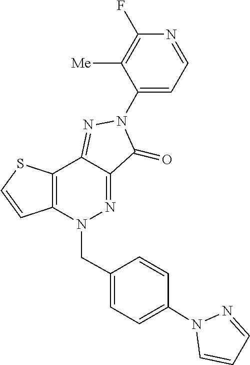 [SMILES: Cc1c(-n2nc3c4sccc4n(Cc4ccc(-n5cccn5)cc4)nc-3c2=O)ccnc1F] |
| A41 | 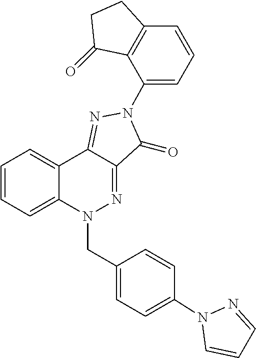 [SMILES: O=C1CCc2cccc(-n3nc4c5ccccc5n(Cc5ccc(-n6cccn6)cc5)nc-4c3=O)c21] |
| A42 | 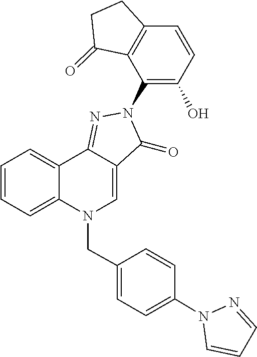 |
| A43 | 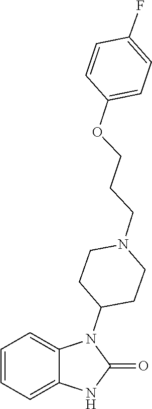 [SMILES: O=c1[nH]c2ccccc2n1C1CCN(CCCOc2ccc(F)cc2)CC1] |
| A44 | 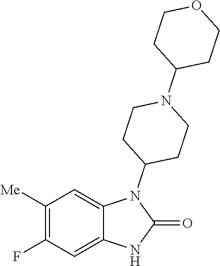 [SMILES: Cc1cc2c(cc1F)[nH]c(=O)n2C1CCN(C2CCOCC2)CC1] |
| A45 | 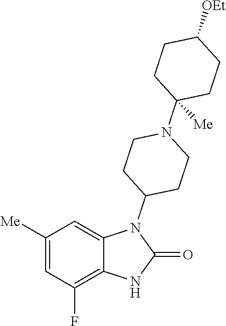 [SMILES: CCO[C@H]1CC[C@](C)(N2CCC(n3c(=O)[nH]c4c(F)cc(C)cc43)CC2)CC1] |
| A46 | 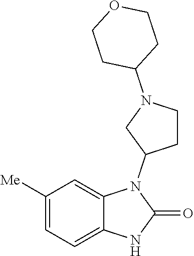 [SMILES: Cc1ccc2[nH]c(=O)n(C3CCN(C4CCOCC4)C3)c2c1] |
| A47 | 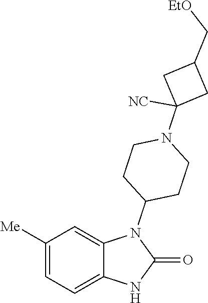 [SMILES: CCOCC1CC(C#N)(N2CCC(n3c(=O)[nH]c4ccc(C)cc43)CC2)C1] |
| A48 | 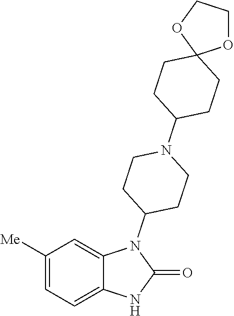 [SMILES: Cc1ccc2[nH]c(=O)n(C3CCN(C4CCC5(CC4)OCCO5)CC3)c2c1] |
| A49 | 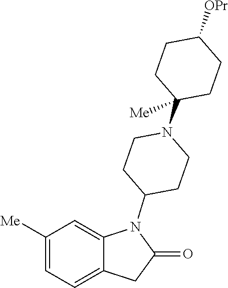 |
| A50 | 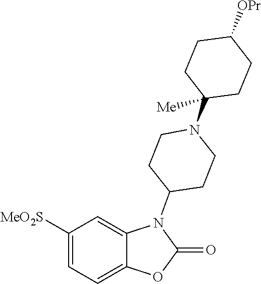 |
| A51 | 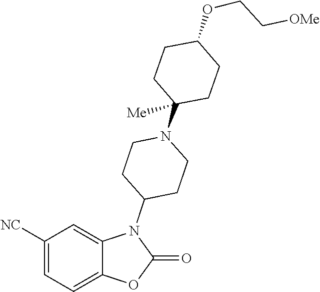 |

The synthesis of compounds A1-A51 is described in US20100216840, WO2009106534, WO2009124882, WO22090124883, TW200840814, WO20090134668, EP2312949, EP2355659, WO2009102558, WO2010042347, US20110112077, WO2011149801, WO2010059773, WO2012003147, WO2011075371, WO2010096338 WO2011049731, WO2007036718, WO2007107567, WO20081197712, WO2008119720 WO2009119716, WO2009037294, Bioorg and Med Chem Lett, 2012, 20, 533-537, Bioorg and Med Chem Lett, 2011, 21, 1710-1715, J of Medicinal Chem: 2011, 13, 4773-4780, ACS Med Chem Lett, 2010, 1, 244-248. The disclosure of each of the foregoing documents is incorporated by reference herein in their entirety.

### NMDA Receptor Positive Allosteric Modulators

The NMDA receptor positive allosteric modulator (PAM) useful in the pharmaceutical compositions, kits and methods of the disclosure is a compound that is a positive modulator of synaptic and/or extrasynaptic NMDA receptors. NMDA PAMs are known to those skilled in the art. Therefore, any NMDA PAM may be useful in the compositions, kits and methods of the present disclosure.

In some embodiments, the NMDA PAM includes, but is not limited to phenanthrene carboxylic acids, naphthoic acids, benzenesulfonamides, thiazoles, pyrrolidinones, tetrahydroisoquinolines, neuroactive steroids, and pharmaceutically acceptable salts thereof.

In some embodiments, the NMDA PAM is a phenanthrene carboxylic acid or pharmaceutically acceptable salt thereof Exemplary phenanthrene carboxylic acids include but are not limited to BP512 or pharmaceutically acceptable salt thereof.

In some embodiments, the NMDA PAM is a naphthoic acid or pharmaceutically acceptable salt thereof. In some embodiments, the naphthoic acid include but are not limited to or pharmaceutically acceptable salt thereof.

In some embodiments, the NMDA PAM is a benzenesulfonamide or pharmaceutically acceptable salt thereof. In some embodiments, the benzenesulfonamide include but are not limited to GNE-9278 or pharmaceutically acceptable salt thereof.

In some embodiments, the NMDA PAM is a thiazole or pharmaceutically acceptable salt thereof. In some embodiments, the thiazole include but are not limited to GNE-3476 or pharmaceutically acceptable salt thereof.

In some embodiments, the NMDA PAM is a pyrrolidinone or pharmaceutically acceptable salt thereof. In some embodiments, the pyrrolidinone include but are not limited to PYD-111 or PYD-106 or pharmaceutically acceptable salts thereof. In some embodiments, the pyrrolidinone is PYD-111 or pharmaceutically acceptable salts thereof. In some embodiments, the pyrrolidinone is PYD-106 or pharmaceutically acceptable salts thereof.

In some embodiments, the NMDA PAM is a tetrahydroisoquinolines or pharmaceutically acceptable salts thereof. In some embodiments, the tetrahydroisoquinoline include but are not limited to CIQ or a pharmaceutically acceptable salt thereof.

In some embodiments, the NMDA PAM is a neuroactive steroid. In some embodiments, the neuroactive steroid includes but is not limited to any one of compounds B1-B659 as set forth in Table 2, or pharmaceutically acceptable salts thereof. In some embodiments, the neuroactive steroid is any one of compounds B1-B659 as set forth in Table 2.

TABLE 2

| No | Structure |
|---|---|
| B1 | 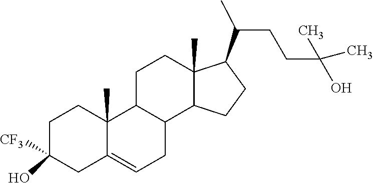 [SMILES: CC(CCC(C)(C)O)[C@H]1CCC2C3CC=C4C[C@](O)(C(F)(F)F)CC[C@]4(C)C3CC[C@@]21C] |
| B2 | 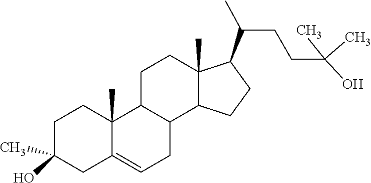 [SMILES: CC(CCC(C)(C)O)[C@H]1CCC2C3CC=C4C[C@@](C)(O)CC[C@]4(C)C3CC[C@@]21C] |
| B3 | 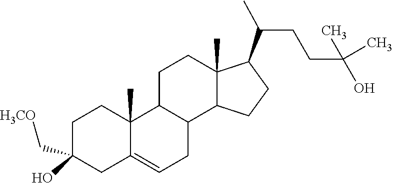 [SMILES: COC[C@]1(O)CC[C@@]2(C)C(=CCC3C2CC[C@@]2(C)C3CC[C@@H]2C(C)CCC(C)(C)O)C1] |
| B4 | 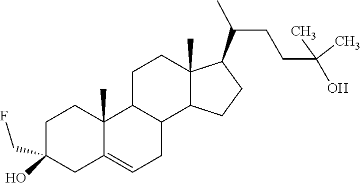 [SMILES: CC(CCC(C)(C)O)[C@H]1CCC2C3CC=C4C[C@](O)(CF)CC[C@]4(C)C3CC[C@@]21C] |
| B5 | 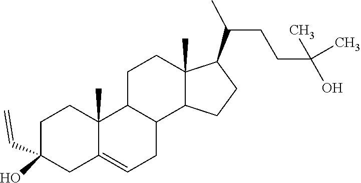 [SMILES: C=C[C@]1(O)CC[C@@]2(C)C(=CCC3C2CC[C@@]2(C)C3CC[C@@H]2C(C)CCC(C)(C)O)C1] |
| B6 | 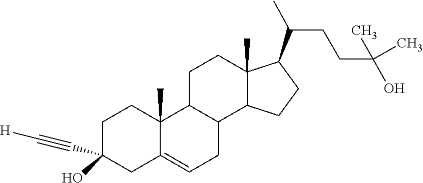 [SMILES: C#C[C@]1(O)CC[C@@]2(C)C(=CCC3C2CC[C@@]2(C)C3CC[C@@H]2C(C)CCC(C)(C)O)C1] |
| B7 | 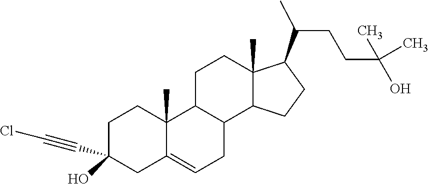 [SMILES: CC(CCC(C)(C)O)[C@H]1CCC2C3CC=C4C[C@](O)(C#CCl)CC[C@]4(C)C3CC[C@@]21C] |
| B8 | 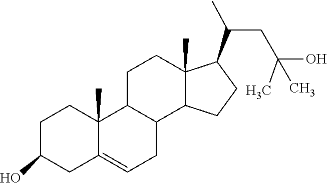 [SMILES: CC(CC(C)(C)O)[C@H]1CCC2C3CC=C4C[C@@H](O)CC[C@]4(C)C3CC[C@@]21C] |
| B9 | 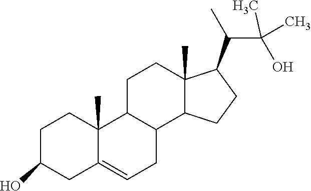 [SMILES: CC([C@H]1CCC2C3CC=C4C[C@@H](O)CC[C@]4(C)C3CC[C@@]21C)C(C)(C)O] |
| B10 | 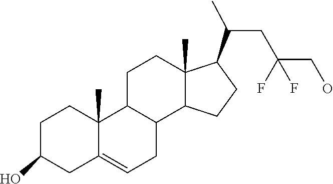 [SMILES: CC(CC(F)(F)CO)[C@H]1CCC2C3CC=C4C[C@@H](O)CC[C@]4(C)C3CC[C@@]21C] |
| B11 | 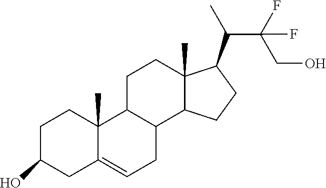 [SMILES: CC([C@H]1CCC2C3CC=C4C[C@@H](O)CC[C@]4(C)C3CC[C@@]21C)C(F)(F)CO] |
| B12 | 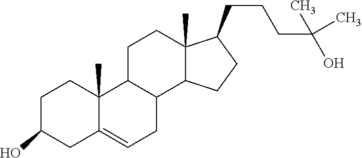 [SMILES: CC(C)(O)CCC[C@H]1CCC2C3CC=C4C[C@@H](O)CC[C@]4(C)C3CC[C@@]21C] |
| B13 | 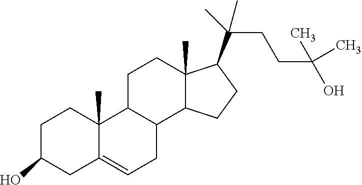 [SMILES: CC(C)(O)CCC(C)(C)[C@H]1CCC2C3CC=C4C[C@@H](O)CC[C@]4(C)C3CC[C@@]21C] |
| B14 | 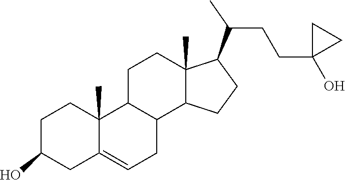 [SMILES: CC(CCC1(O)CC1)[C@H]1CCC2C3CC=C4C[C@@H](O)CC[C@]4(C)C3CC[C@@]21C] |
| B15 | 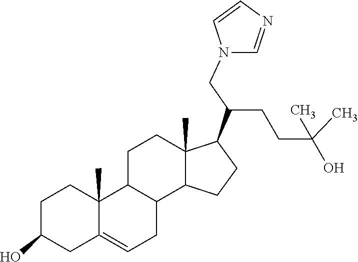 [SMILES: CC(C)(O)CCC(Cn1ccnc1)[C@H]1CCC2C3CC=C4C[C@@H](O)CC[C@]4(C)C3CC[C@@]21C] |
| B16 | 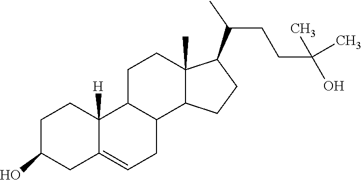 [SMILES: CC(CCC(C)(C)O)[C@H]1CCC2C3CC=C4C[C@@H](O)CC[C@@H]4C3CC[C@@]21C] |
| B17 | 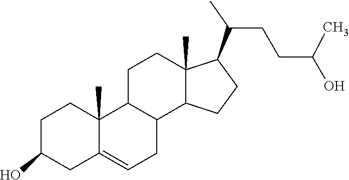 [SMILES: CC(O)CCC(C)[C@H]1CCC2C3CC=C4C[C@@H](O)CC[C@]4(C)C3CC[C@@]21C] |
| B18 | 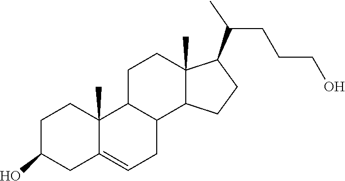 [SMILES: CC(CCCO)[C@H]1CCC2C3CC=C4C[C@@H](O)CC[C@]4(C)C3CC[C@@]21C] |
| B19 | 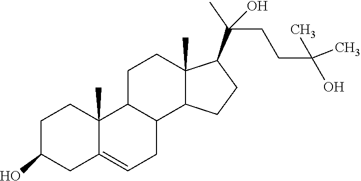 [SMILES: CC(C)(O)CCC(C)(O)[C@H]1CCC2C3CC=C4C[C@@H](O)CC[C@]4(C)C3CC[C@@]21C] |
| B20 | 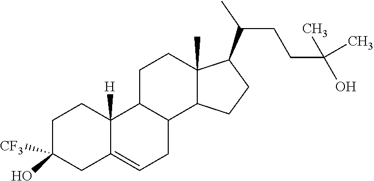 [SMILES: CC(CCC(C)(C)O)[C@H]1CCC2C3CC=C4C[C@](O)(C(F)(F)F)CC[C@@H]4C3CC[C@@]21C] |
| B21 | 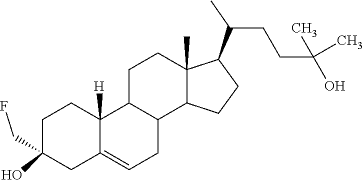 [SMILES: CC(CCC(C)(C)O)[C@H]1CCC2C3CC=C4C[C@](O)(CF)CC[C@@H]4C3CC[C@@]21C] |
| B22 | 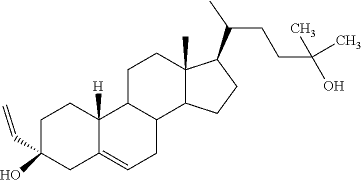 [SMILES: C=C[C@]1(O)CC[C@H]2C(=CCC3C2CC[C@@]2(C)C3CC[C@@H]2C(C)CCC(C)(C)O)C1] |
| B23 | 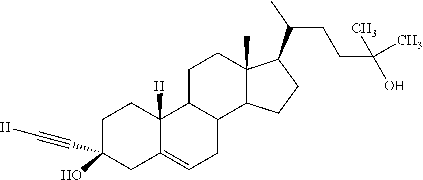 [SMILES: C#C[C@]1(O)CC[C@H]2C(=CCC3C2CC[C@@]2(C)C3CC[C@@H]2C(C)CCC(C)(C)O)C1] |
| B24 | 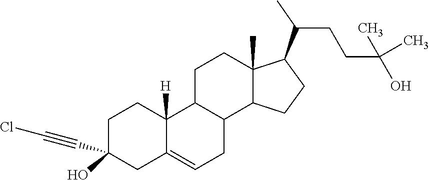 [SMILES: CC(CCC(C)(C)O)[C@H]1CCC2C3CC=C4C[C@](O)(C#CCl)CC[C@@H]4C3CC[C@@]21C] |
| B25 | 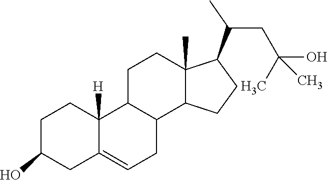 [SMILES: CC(CC(C)(C)O)[C@H]1CCC2C3CC=C4C[C@@H](O)CC[C@@H]4C3CC[C@@]21C] |
| B26 | 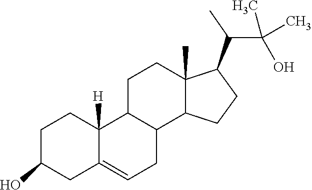 [SMILES: CC([C@H]1CCC2C3CC=C4C[C@@H](O)CC[C@@H]4C3CC[C@@]21C)C(C)(C)O] |
| B27 | 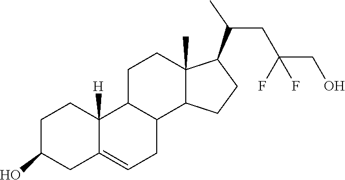 [SMILES: CC(CC(F)(F)CO)[C@H]1CCC2C3CC=C4C[C@@H](O)CC[C@@H]4C3CC[C@@]21C] |
| B28 | 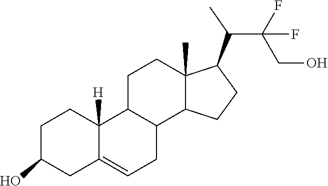 [SMILES: CC([C@H]1CCC2C3CC=C4C[C@@H](O)CC[C@@H]4C3CC[C@@]21C)C(F)(F)CO] |
| B29 | 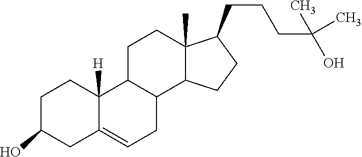 [SMILES: CC(C)(O)CCC[C@H]1CCC2C3CC=C4C[C@@H](O)CC[C@@H]4C3CC[C@@]21C] |
| B30 | 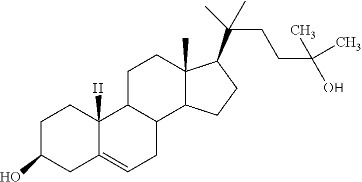 [SMILES: CC(C)(O)CCC(C)(C)[C@H]1CCC2C3CC=C4C[C@@H](O)CC[C@@H]4C3CC[C@@]21C] |
| B31 | 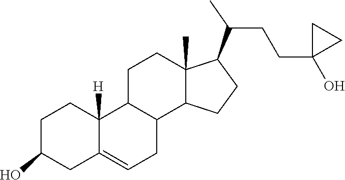 [SMILES: CC(CCC1(O)CC1)[C@H]1CCC2C3CC=C4C[C@@H](O)CC[C@@H]4C3CC[C@@]21C] |
| B32 | 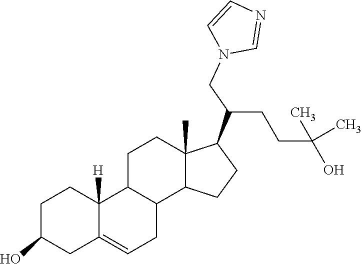 [SMILES: CC(C)(O)CCC(Cn1ccnc1)[C@H]1CCC2C3CC=C4C[C@@H](O)CC[C@@H]4C3CC[C@@]21C] |
| B33 | 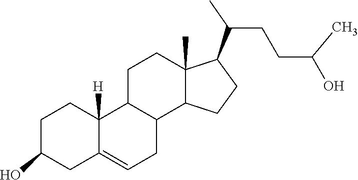 [SMILES: CC(O)CCC(C)[C@H]1CCC2C3CC=C4C[C@@H](O)CC[C@@H]4C3CC[C@@]21C] |
| B34 | 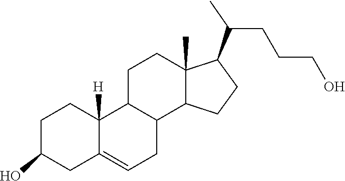 [SMILES: CC(CCCO)[C@H]1CCC2C3CC=C4C[C@@H](O)CC[C@@H]4C3CC[C@@]21C] |
| B35 | 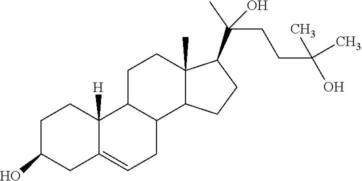 [SMILES: CC(C)(O)CCC(C)(O)[C@H]1CCC2C3CC=C4C[C@@H](O)CC[C@@H]4C3CC[C@@]21C] |
| B36 | 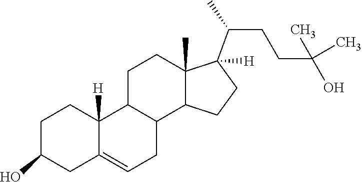 [SMILES: C[C@H](CCC(C)(C)O)[C@H]1CCC2C3CC=C4C[C@@H](O)CC[C@@H]4C3CC[C@@]21C] |
| B37 | 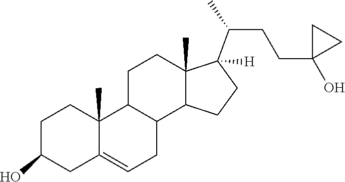 [SMILES: C[C@H](CCC1(O)CC1)[C@H]1CCC2C3CC=C4C[C@@H](O)CC[C@]4(C)C3CC[C@@]21C] |
| B38 | 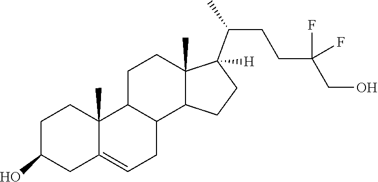 [SMILES: C[C@H](CCC(F)(F)CO)[C@H]1CCC2C3CC=C4C[C@@H](O)CC[C@]4(C)C3CC[C@@]21C] |
| B39 | 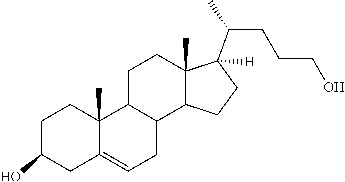 [SMILES: C[C@H](CCCO)[C@H]1CCC2C3CC=C4C[C@@H](O)CC[C@]4(C)C3CC[C@@]21C] |
| B40 | 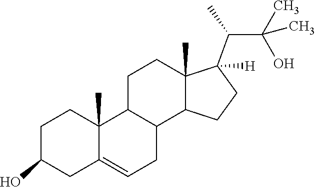 [SMILES: C[C@@H]([C@H]1CCC2C3CC=C4C[C@@H](O)CC[C@]4(C)C3CC[C@@]21C)C(C)(C)O] |
| B41 | 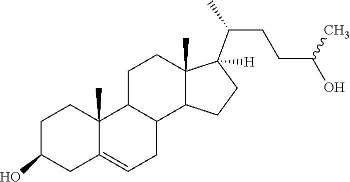 [SMILES: CC(O)CC[C@@H](C)[C@H]1CCC2C3CC=C4C[C@@H](O)CC[C@]4(C)C3CC[C@@]21C] |
| B42 | 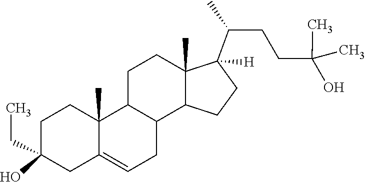 [SMILES: CC[C@]1(O)CC[C@@]2(C)C(=CCC3C2CC[C@@]2(C)C3CC[C@@H]2[C@H](C)CCC(C)(C)O)C1] |
| B43 | 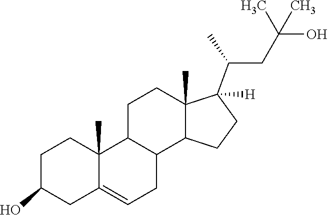 [SMILES: C[C@H](CC(C)(C)O)[C@H]1CCC2C3CC=C4C[C@@H](O)CC[C@]4(C)C3CC[C@@]21C] |
| B44 | 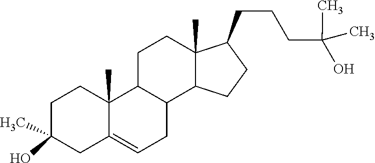 [SMILES: CC(C)(O)CCC[C@H]1CCC2C3CC=C4C[C@@](C)(O)CC[C@]4(C)C3CC[C@@]21C] |
| B45 | 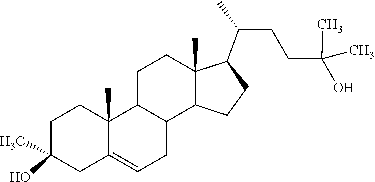 |
| B46 | 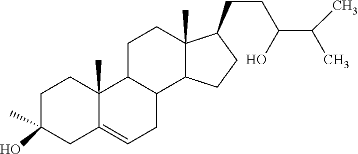 [SMILES: CC(C)C(O)CC[C@H]1CCC2C3CC=C4C[C@@](C)(O)CC[C@]4(C)C3CC[C@@]21C] |
| B47 | 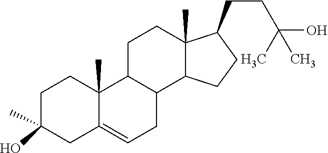 [SMILES: CC(C)(O)CC[C@H]1CCC2C3CC=C4C[C@@](C)(O)CC[C@]4(C)C3CC[C@@]21C] |
| B48 | 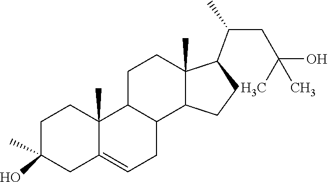 |
| B49 | 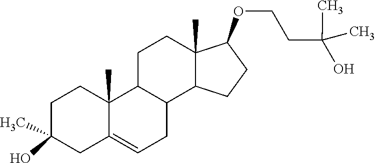 [SMILES: CC(C)(O)CCO[C@H]1CCC2C3CC=C4C[C@@](C)(O)CC[C@]4(C)C3CC[C@@]21C] |
| B50 | 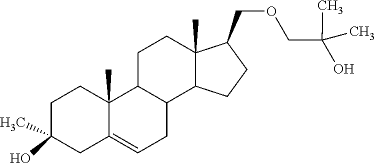 [SMILES: CC(C)(O)COC[C@H]1CCC2C3CC=C4C[C@@](C)(O)CC[C@]4(C)C3CC[C@@]21C] |
| B51 | 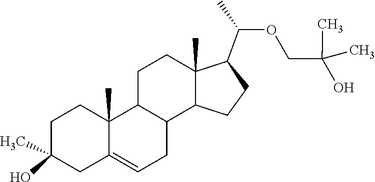 |
| B52 | 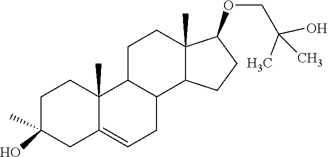 [SMILES: CC(C)(O)CO[C@H]1CCC2C3CC=C4C[C@@](C)(O)CC[C@]4(C)C3CC[C@@]21C] |
| B53 | 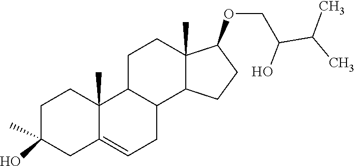 [SMILES: CC(C)C(O)CO[C@H]1CCC2C3CC=C4C[C@@](C)(O)CC[C@]4(C)C3CC[C@@]21C] |
| B54 | 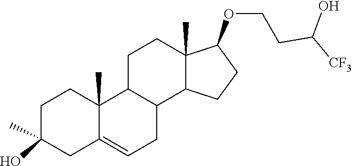 [SMILES: C[C@]1(O)CC[C@@]2(C)C(=CCC3C2CC[C@@]2(C)C3CC[C@@H]2OCCC(O)C(F)(F)F)C1] |
| B55 | 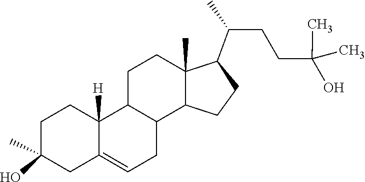 |
| B56 | 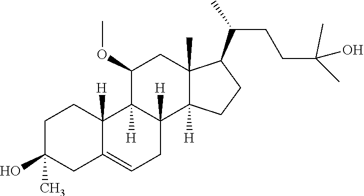 |
| B57 | 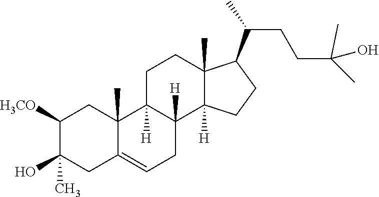 |
| B58 | 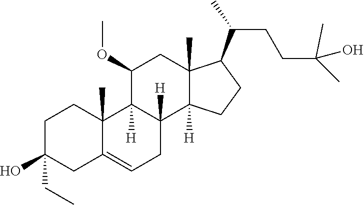 |
| B59 | 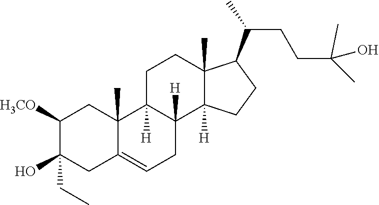 |
| B60 | 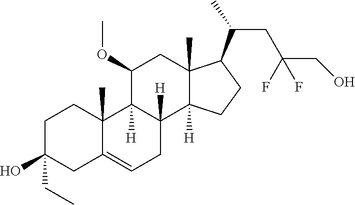 |
| B61 | 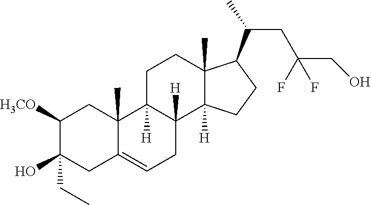 |
| B62 | 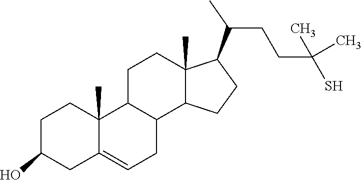 [SMILES: CC(CCC(C)(C)S)[C@H]1CCC2C3CC=C4C[C@@H](O)CC[C@]4(C)C3CC[C@@]21C] |
| B63 | 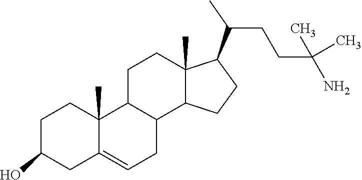 [SMILES: CC(CCC(C)(C)N)[C@H]1CCC2C3CC=C4C[C@@H](O)CC[C@]4(C)C3CC[C@@]21C] |
| B64 | 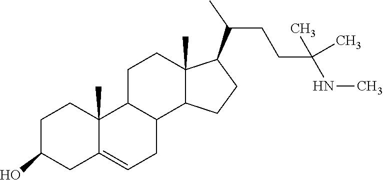 [SMILES: CNC(C)(C)CCC(C)[C@H]1CCC2C3CC=C4C[C@@H](O)CC[C@]4(C)C3CC[C@@]21C] |
| B65 | 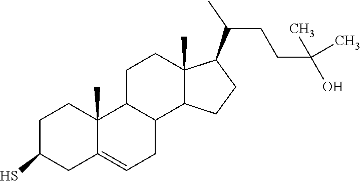 [SMILES: CC(CCC(C)(C)O)[C@H]1CCC2C3CC=C4C[C@@H](S)CC[C@]4(C)C3CC[C@@]21C] |
| B66 | 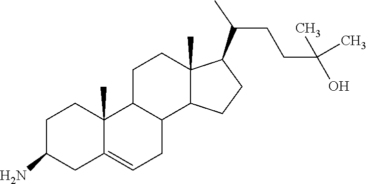 [SMILES: CC(CCC(C)(C)O)[C@H]1CCC2C3CC=C4C[C@@H](N)CC[C@]4(C)C3CC[C@@]21C] |
| B67 | 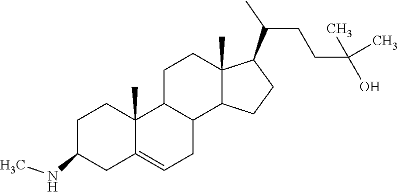 [SMILES: CN[C@H]1CC[C@@]2(C)C(=CCC3C2CC[C@@]2(C)C3CC[C@@H]2C(C)CCC(C)(C)O)C1] |
| B68 | 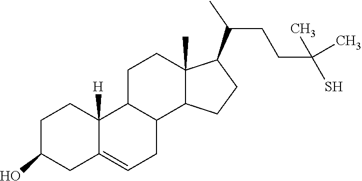 [SMILES: CC(CCC(C)(C)S)[C@H]1CCC2C3CC=C4C[C@@H](O)CC[C@@H]4C3CC[C@@]21C] |
| B69 | 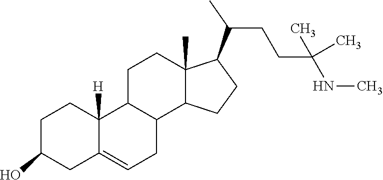 [SMILES: CNC(C)(C)CCC(C)[C@H]1CCC2C3CC=C4C[C@@H](O)CC[C@@H]4C3CC[C@@]21C] |
| B70 | 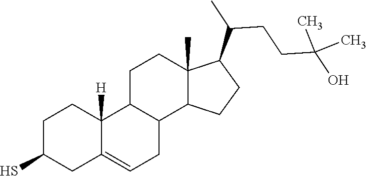 [SMILES: CC(CCC(C)(C)O)[C@H]1CCC2C3CC=C4C[C@@H](S)CC[C@@H]4C3CC[C@@]21C] |
| B71 | 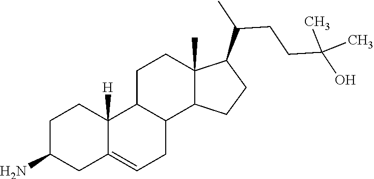 [SMILES: CC(CCC(C)(C)O)[C@H]1CCC2C3CC=C4C[C@@H](N)CC[C@@H]4C3CC[C@@]21C] |
| B72 | 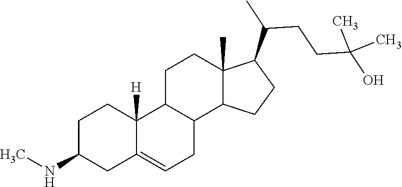 [SMILES: CN[C@H]1CC[C@H]2C(=CCC3C2CC[C@@]2(C)C3CC[C@@H]2C(C)CCC(C)(C)O)C1] |
| B73 | 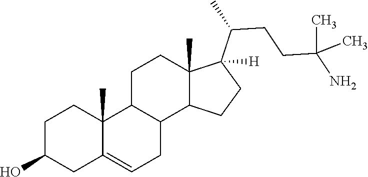 [SMILES: C[C@H](CCC(C)(C)N)[C@H]1CCC2C3CC=C4C[C@@H](O)CC[C@]4(C)C3CC[C@@]21C] |
| B74 | 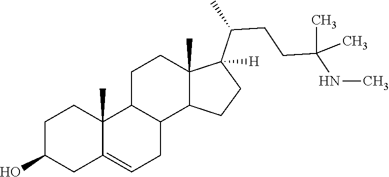 [SMILES: CNC(C)(C)CC[C@@H](C)[C@H]1CCC2C3CC=C4C[C@@H](O)CC[C@]4(C)C3CC[C@@]21C] |
| B75 | 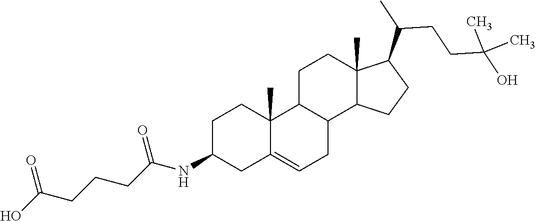 [SMILES: CC(CCC(C)(C)O)[C@H]1CCC2C3CC=C4C[C@@H](NC(=O)CCCC(=O)O)CC[C@]4(C)C3CC[C@@]21C] |
| B76 | 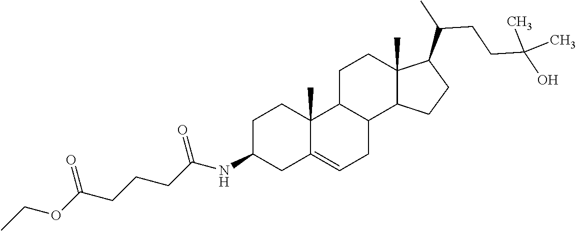 [SMILES: CCOC(=O)CCCC(=O)N[C@H]1CC[C@@]2(C)C(=CCC3C2CC[C@@]2(C)C3CC[C@@H]2C(C)CCC(C)(C)O)C1] |
| B77 | 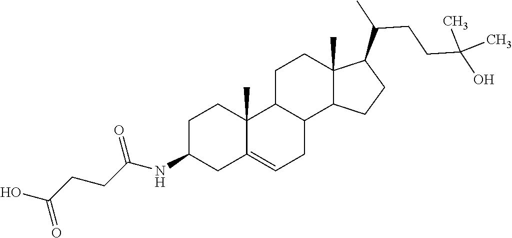 [SMILES: CC(CCC(C)(C)O)[C@H]1CCC2C3CC=C4C[C@@H](NC(=O)CCC(=O)O)CC[C@]4(C)C3CC[C@@]21C] |
| B78 | 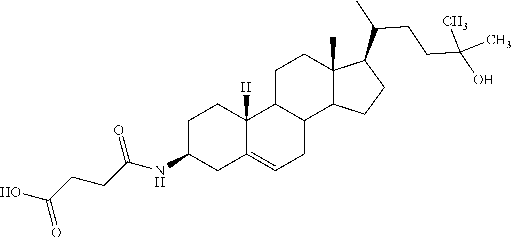 [SMILES: CC(CCC(C)(C)O)[C@H]1CCC2C3CC=C4C[C@@H](NC(=O)CCC(=O)O)CC[C@@H]4C3CC[C@@]21C] |
| B79 | 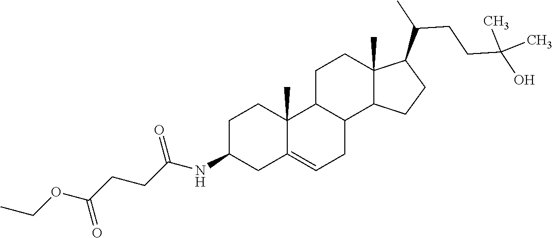 [SMILES: CCOC(=O)CCC(=O)N[C@H]1CC[C@@]2(C)C(=CCC3C2CC[C@@]2(C)C3CC[C@@H]2C(C)CCC(C)(C)O)C1] |
| B80 | 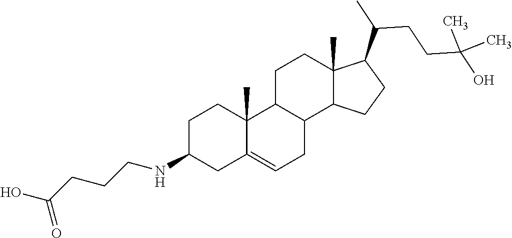 [SMILES: CC(CCC(C)(C)O)[C@H]1CCC2C3CC=C4C[C@@H](NCCCC(=O)O)CC[C@]4(C)C3CC[C@@]21C] |
| B81 | 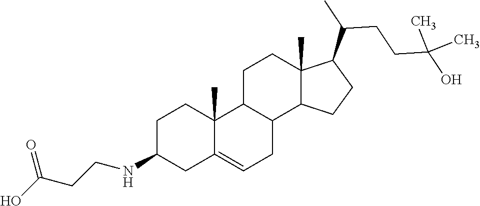 [SMILES: CC(CCC(C)(C)O)[C@H]1CCC2C3CC=C4C[C@@H](NCCC(=O)O)CC[C@]4(C)C3CC[C@@]21C] |
| B82 | 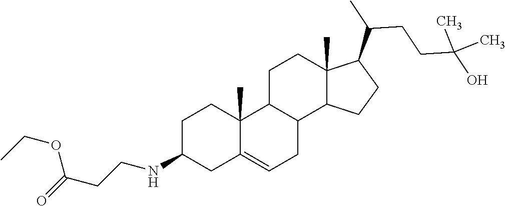 [SMILES: CCOC(=O)CCN[C@H]1CC[C@@]2(C)C(=CCC3C2CC[C@@]2(C)C3CC[C@@H]2C(C)CCC(C)(C)O)C1] |
| B83 | 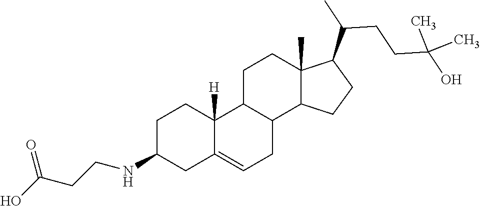 [SMILES: CC(CCC(C)(C)O)[C@H]1CCC2C3CC=C4C[C@@H](NCCC(=O)O)CC[C@@H]4C3CC[C@@]21C] |
| B84 | 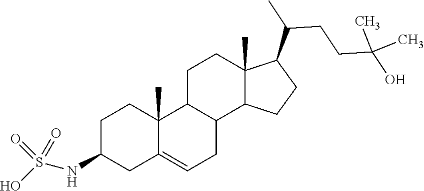 [SMILES: CC(CCC(C)(C)O)[C@H]1CCC2C3CC=C4C[C@@H](NS(=O)(=O)O)CC[C@]4(C)C3CC[C@@]21C] |
| B85 | 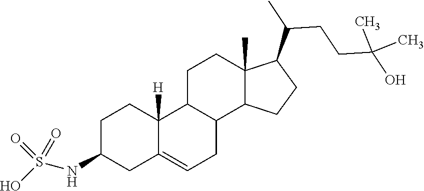 [SMILES: CC(CCC(C)(C)O)[C@H]1CCC2C3CC=C4C[C@@H](NS(=O)(=O)O)CC[C@@H]4C3CC[C@@]21C] |
| B86 | 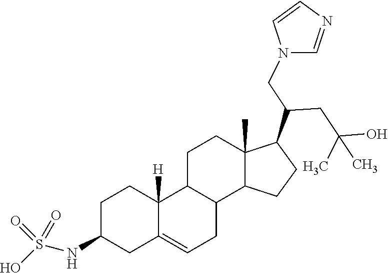 [SMILES: CC(C)(O)CC(Cn1ccnc1)[C@H]1CCC2C3CC=C4C[C@@H](NS(=O)(=O)O)CC[C@@H]4C3CC[C@@]21C] |
| B87 | 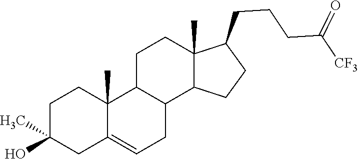 [SMILES: C[C@]1(O)CC[C@@]2(C)C(=CCC3C2CC[C@@]2(C)C3CC[C@@H]2CCCC(=O)C(F)(F)F)C1] |
| B88 | 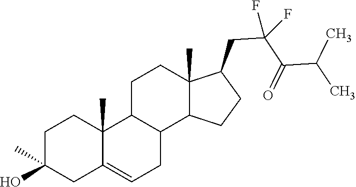 [SMILES: CC(C)C(=O)C(F)(F)C[C@H]1CCC2C3CC=C4C[C@@](C)(O)CC[C@]4(C)C3CC[C@@]21C] |
| B89 | 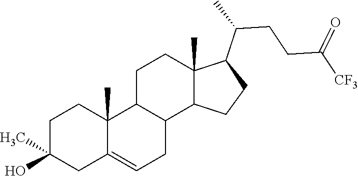 |
| B90 | 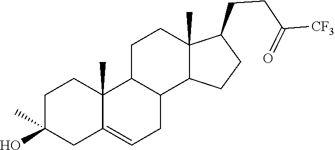 [SMILES: C[C@]1(O)CC[C@@]2(C)C(=CCC3C2CC[C@@]2(C)C3CC[C@@H]2CCC(=O)C(F)(F)F)C1] |
| B91 | 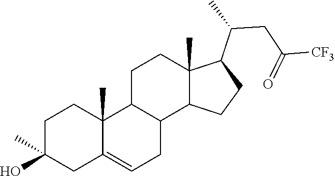 |
| B92 | 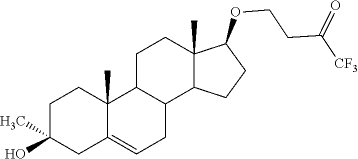 [SMILES: C[C@]1(O)CC[C@@]2(C)C(=CCC3C2CC[C@@]2(C)C3CC[C@@H]2OCCC(=O)C(F)(F)F)C1] |
| B93 | 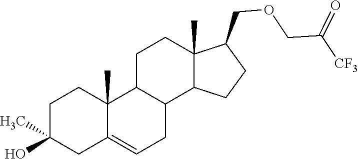 [SMILES: C[C@]1(O)CC[C@@]2(C)C(=CCC3C2CC[C@@]2(C)C3CC[C@@H]2COCC(=O)C(F)(F)F)C1] |
| B94 | 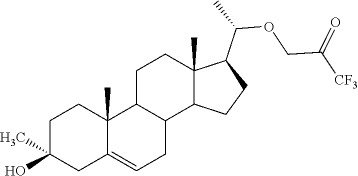 |
| B95 | 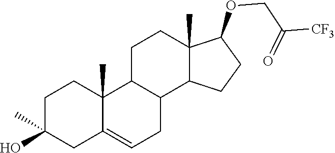 [SMILES: C[C@]1(O)CC[C@@]2(C)C(=CCC3C2CC[C@@]2(C)C3CC[C@@H]2OCC(=O)C(F)(F)F)C1] |
| B96 | 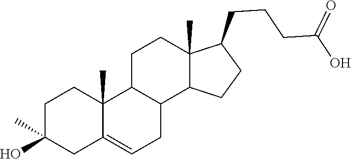 [SMILES: C[C@]1(O)CC[C@@]2(C)C(=CCC3C2CC[C@@]2(C)C3CC[C@@H]2CCCC(=O)O)C1] |
| B97 | 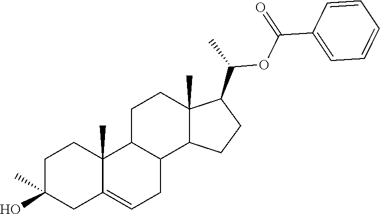 [SMILES: C[C@H](OC(=O)c1ccccc1)[C@H]1CCC2C3CC=C4C[C@@](C)(O)CC[C@]4(C)C3CC[C@@]21C] |
| B98 | 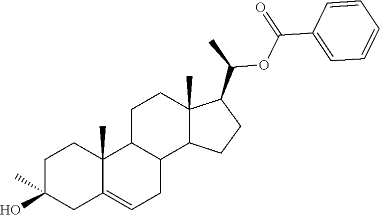 [SMILES: C[C@@H](OC(=O)c1ccccc1)[C@H]1CCC2C3CC=C4C[C@@](C)(O)CC[C@]4(C)C3CC[C@@]21C] |
| B99 | 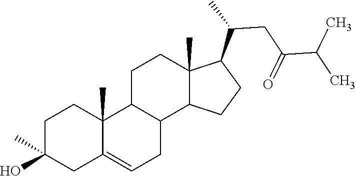 [SMILES: CC(C)C(=O)C[C@@H](C)[C@H]1CCC2C3CC=C4C[C@@](C)(O)CC[C@]4(C)C3CC[C@@]21C] |
| B100 | 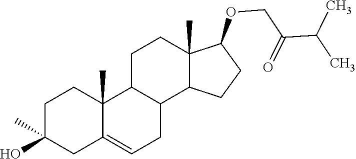 [SMILES: CC(C)C(=O)CO[C@H]1CCC2C3CC=C4C[C@@](C)(O)CC[C@]4(C)C3CC[C@@]21C] |
| B101 | 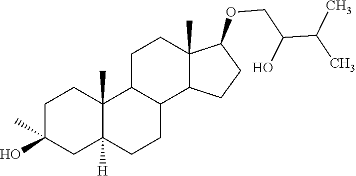 [SMILES: CC(C)C(O)CO[C@H]1CCC2C3CC[C@H]4C[C@@](C)(O)CC[C@]4(C)C3CC[C@@]21C] |
| B102 | 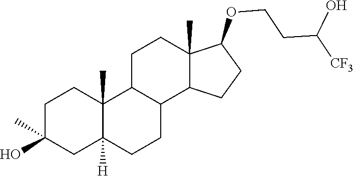 [SMILES: C[C@]1(O)CC[C@]2(C)C3CC[C@@]4(C)C(CC[C@@H]4OCCC(O)C(F)(F)F)C3CC[C@H]2C1] |
| B103 | 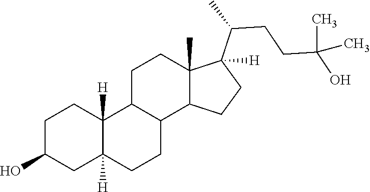 [SMILES: C[C@H](CCC(C)(C)O)[C@H]1CCC2C3CC[C@H]4C[C@@H](O)CC[C@@H]4C3CC[C@@]21C] |
| B104 | 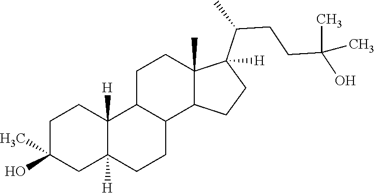 [SMILES: C[C@H](CCC(C)(C)O)[C@H]1CCC2C3CC[C@H]4C[C@@](C)(O)CC[C@@H]4C3CC[C@@]21C] |
| B105 | 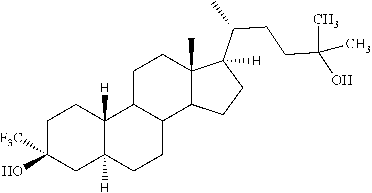 [SMILES: C[C@H](CCC(C)(C)O)[C@H]1CCC2C3CC[C@H]4C[C@](O)(C(F)(F)F)CC[C@@H]4C3CC[C@@]21C] |
| B106 | 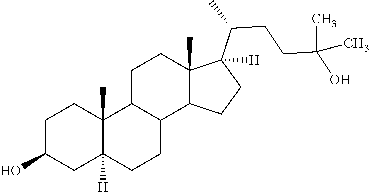 [SMILES: C[C@H](CCC(C)(C)O)[C@H]1CCC2C3CC[C@H]4C[C@@H](O)CC[C@]4(C)C3CC[C@@]21C] |
| B107 | 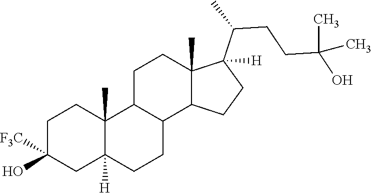 [SMILES: C[C@H](CCC(C)(C)O)[C@H]1CCC2C3CC[C@H]4C[C@](O)(C(F)(F)F)CC[C@]4(C)C3CC[C@@]21C] |
| B108 | 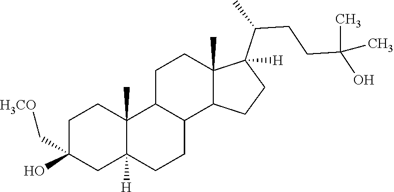 [SMILES: COC[C@]1(O)CC[C@]2(C)C3CC[C@@]4(C)C(CC[C@@H]4[C@H](C)CCC(C)(C)O)C3CC[C@H]2C1] |
| B109 | 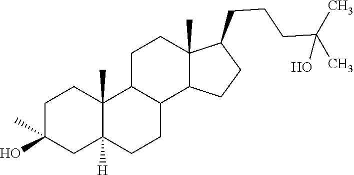 [SMILES: CC(C)(O)CCC[C@H]1CCC2C3CC[C@H]4C[C@@](C)(O)CC[C@]4(C)C3CC[C@@]21C] |
| B110 | 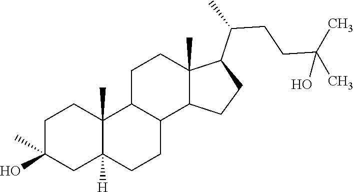 |
| B111 | 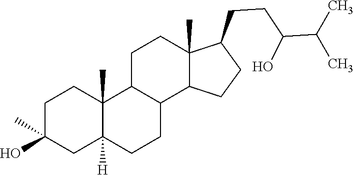 [SMILES: CC(C)C(O)CC[C@H]1CCC2C3CC[C@H]4C[C@@](C)(O)CC[C@]4(C)C3CC[C@@]21C] |
| B112 | 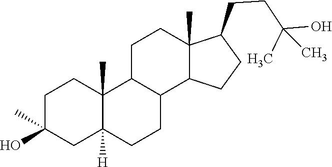 [SMILES: CC(C)(O)CC[C@H]1CCC2C3CC[C@H]4C[C@@](C)(O)CC[C@]4(C)C3CC[C@@]21C] |
| B113 | 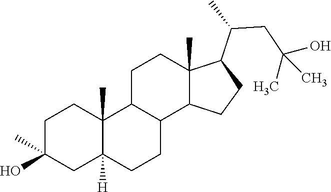 |
| B114 | 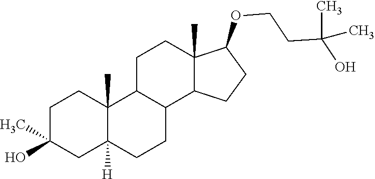 [SMILES: CC(C)(O)CCO[C@H]1CCC2C3CC[C@H]4C[C@@](C)(O)CC[C@]4(C)C3CC[C@@]21C] |
| B115 | 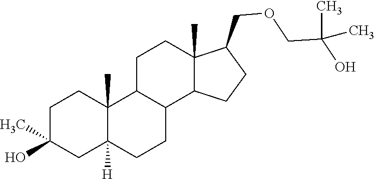 [SMILES: CC(C)(O)COC[C@H]1CCC2C3CC[C@H]4C[C@@](C)(O)CC[C@]4(C)C3CC[C@@]21C] |
| B116 | 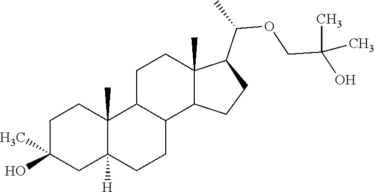 |
| B117 | 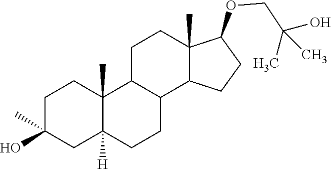 [SMILES: CC(C)(O)CO[C@H]1CCC2C3CC[C@H]4C[C@@](C)(O)CC[C@]4(C)C3CC[C@@]21C] |
| B118 | 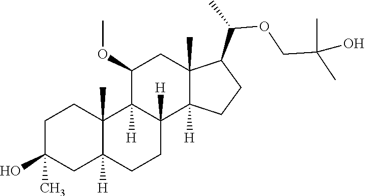 |
| B119 | 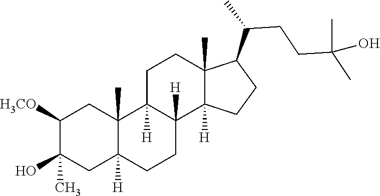 |
| B120 | 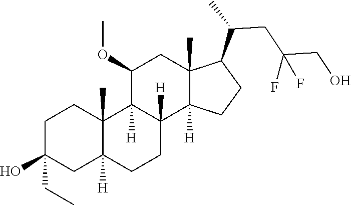 |
| B121 | 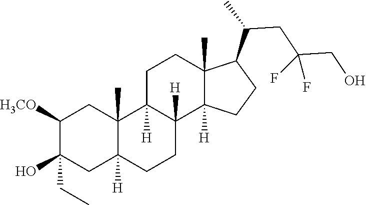 |
| B122 | 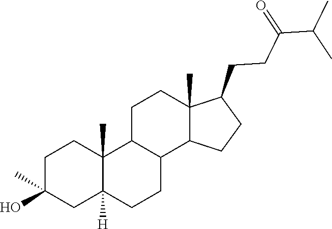 [SMILES: CC(C)C(=O)CC[C@H]1CCC2C3CC[C@H]4C[C@@](C)(O)CC[C@]4(C)C3CC[C@@]21C] |
| B123 | 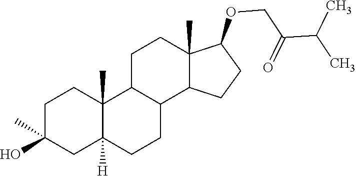 [SMILES: CC(C)C(=O)CO[C@H]1CCC2C3CC[C@H]4C[C@@](C)(O)CC[C@]4(C)C3CC[C@@]21C] |
| B124 | 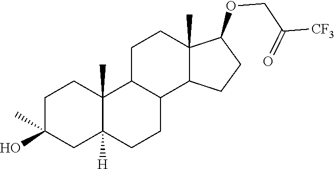 [SMILES: C[C@]1(O)CC[C@]2(C)C3CC[C@@]4(C)C(CC[C@@H]4OCC(=O)C(F)(F)F)C3CC[C@H]2C1] |
| B125 | 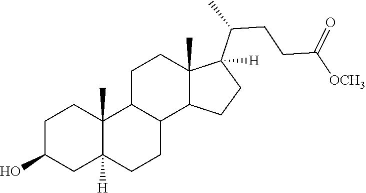 [SMILES: COC(=O)CC[C@@H](C)[C@H]1CCC2C3CC[C@H]4C[C@@H](O)CC[C@]4(C)C3CC[C@@]21C] |
| B126 | 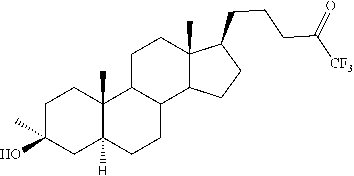 [SMILES: C[C@]1(O)CC[C@]2(C)C3CC[C@@]4(C)C(CC[C@@H]4CCCC(=O)C(F)(F)F)C3CC[C@H]2C1] |
| B127 | 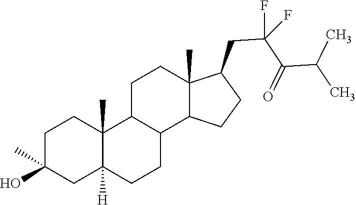 [SMILES: CC(C)C(=O)C(F)(F)C[C@H]1CCC2C3CC[C@H]4C[C@@](C)(O)CC[C@]4(C)C3CC[C@@]21C] |
| B128 | 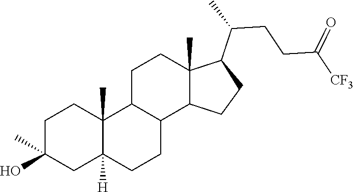 |
| B129 | 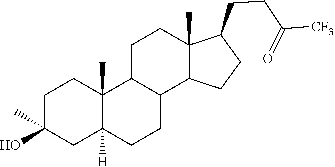 [SMILES: C[C@]1(O)CC[C@]2(C)C3CC[C@@]4(C)C(CC[C@@H]4CCC(=O)C(F)(F)F)C3CC[C@H]2C1] |
| B130 | 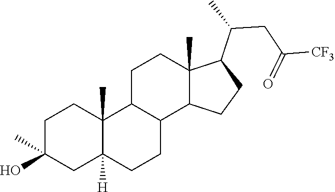 |
| B131 | 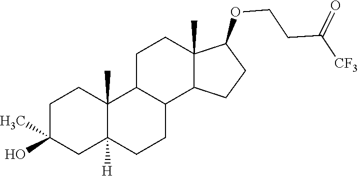 [SMILES: C[C@]1(O)CC[C@]2(C)C3CC[C@@]4(C)C(CC[C@@H]4OCCC(=O)C(F)(F)F)C3CC[C@H]2C1] |
| B132 | 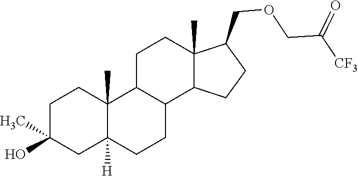 [SMILES: C[C@]1(O)CC[C@]2(C)C3CC[C@@]4(C)C(CC[C@@H]4COCC(=O)C(F)(F)F)C3CC[C@H]2C1] |
| B133 | 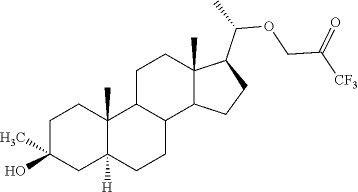 |
| B134 | 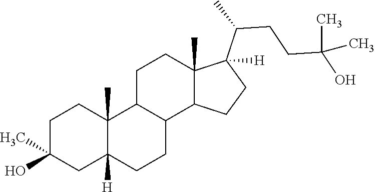 [SMILES: C[C@H](CCC(C)(C)O)[C@H]1CCC2C3CC[C@@H]4C[C@@](C)(O)CC[C@]4(C)C3CC[C@@]21C] |
| B135 | 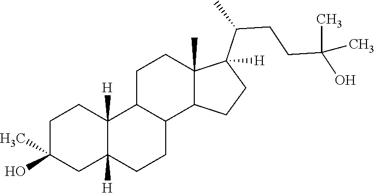 [SMILES: C[C@H](CCC(C)(C)O)[C@H]1CCC2C3CC[C@@H]4C[C@@](C)(O)CC[C@@H]4C3CC[C@@]21C] |
| B136 | 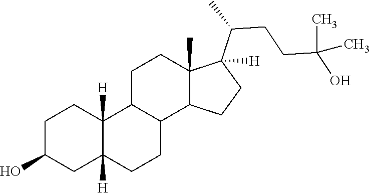 [SMILES: C[C@H](CCC(C)(C)O)[C@H]1CCC2C3CC[C@@H]4C[C@@H](O)CC[C@@H]4C3CC[C@@]21C] |
| B137 | 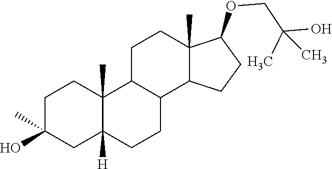 [SMILES: CC(C)(O)CO[C@H]1CCC2C3CC[C@@H]4C[C@@](C)(O)CC[C@]4(C)C3CC[C@@]21C] |
| B138 | 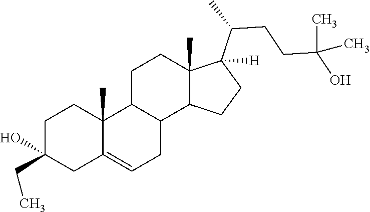 [SMILES: CC[C@@]1(O)CC[C@@]2(C)C(=CCC3C2CC[C@@]2(C)C3CC[C@@H]2[C@H](C)CCC(C)(C)O)C1] |
| B139 | 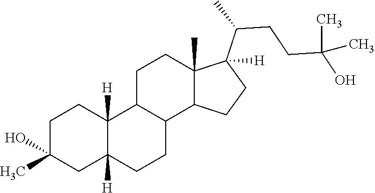 [SMILES: C[C@H](CCC(C)(C)O)[C@H]1CCC2C3CC[C@@H]4C[C@](C)(O)CC[C@@H]4C3CC[C@@]21C] |
| B140 | 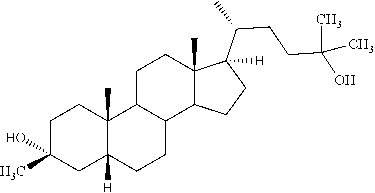 [SMILES: C[C@H](CCC(C)(C)O)[C@H]1CCC2C3CC[C@@H]4C[C@](C)(O)CC[C@]4(C)C3CC[C@@]21C] |
| B141 | 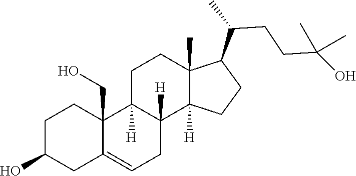 |
| B142 | 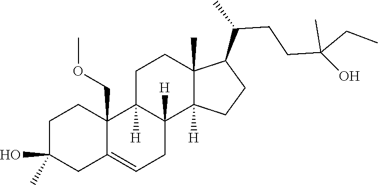 |
| B143 | 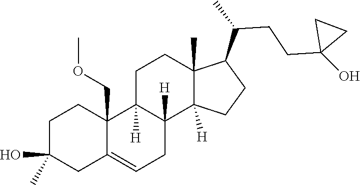 |
| B144 | 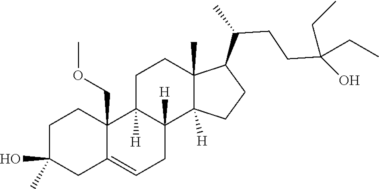 |
| B145 | 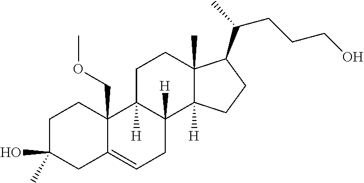 |
| B146 | 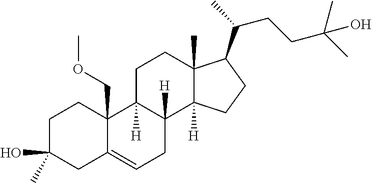 |
| B147 | 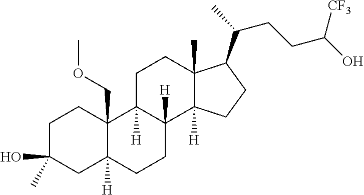 |
| B148 | 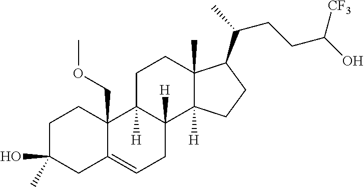 |
| B149 | 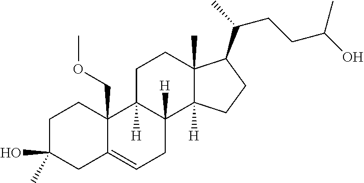 |
| B150 | 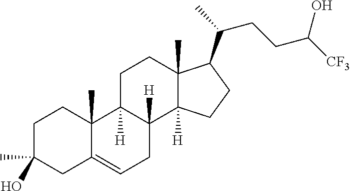 |
| B151 | 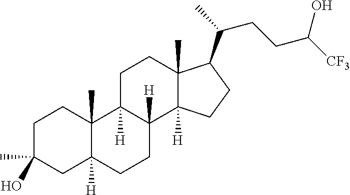 |
| B152 | 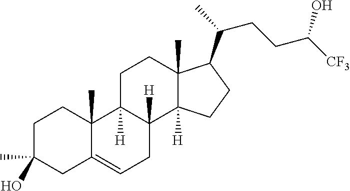 |
| B153 | 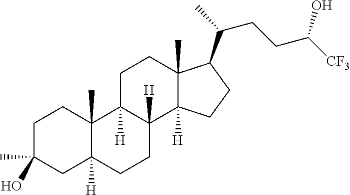 |
| B154 | 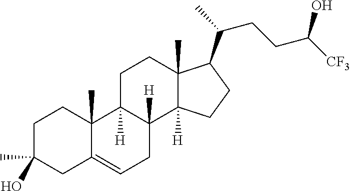 |
| B155 | 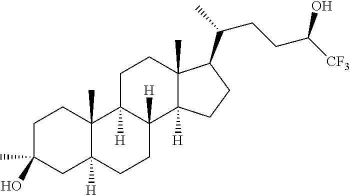 |
| B156 | 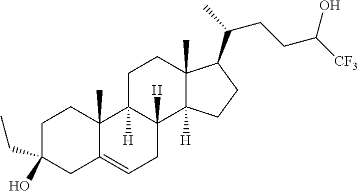 |
| B157 | 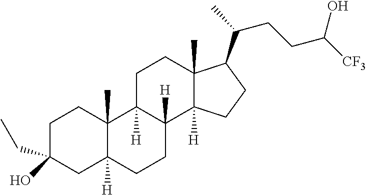 |
| B158 | 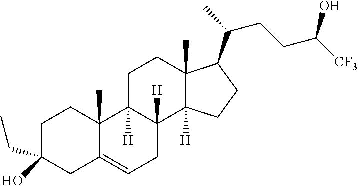 |
| B159 | 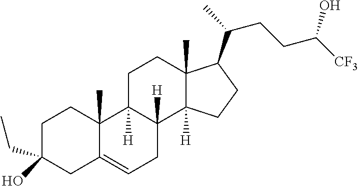 |
| B160 | 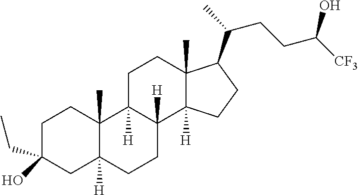 |
| B161 | 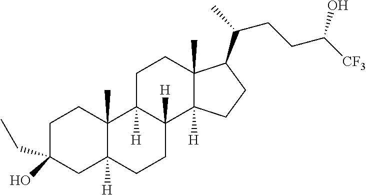 |
| B162 | 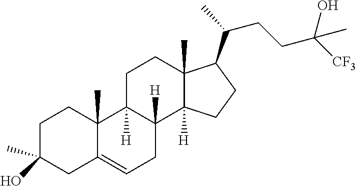 |
| B163 | 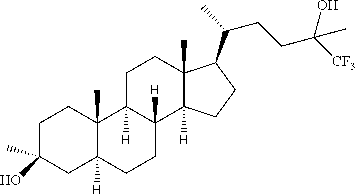 |
| B164 | 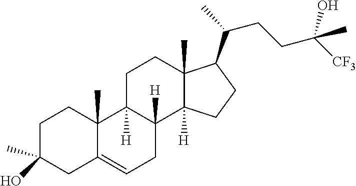 |
| B165 | 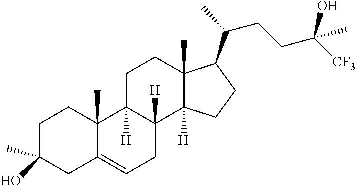 |
| B166 | 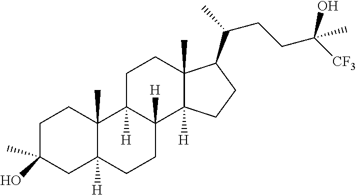 |
| B167 | 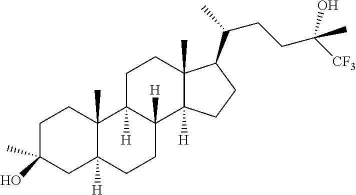 |
| B168 | 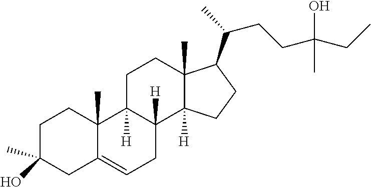 |
| B169 | 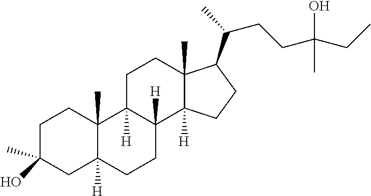 |
| B170 | 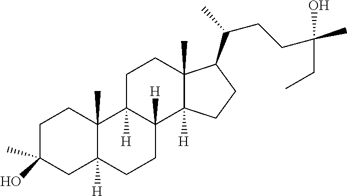 |
| B171 | 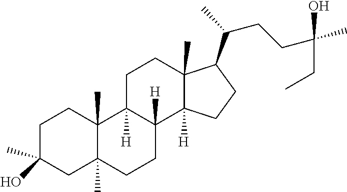 |
| B172 | 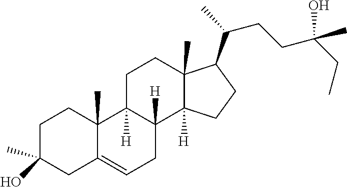 |
| B173 | 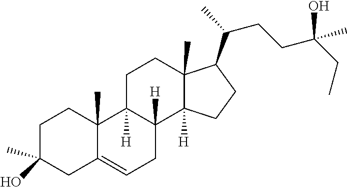 |
| B174 | 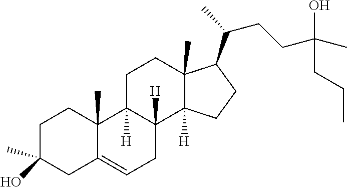 |
| B175 | 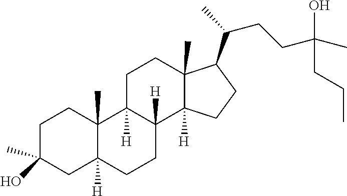 |
| B176 | 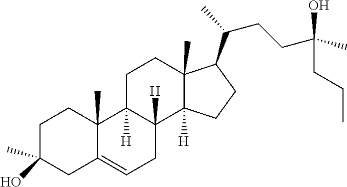 |
| B177 | 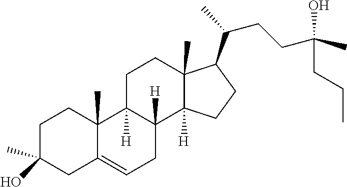 |
| B178 | 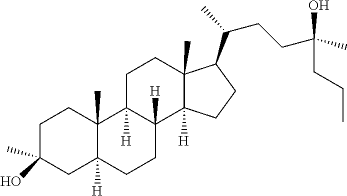 |
| B179 | 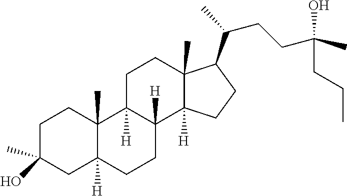 |
| B180 | 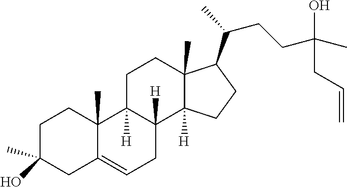 |
| B181 | 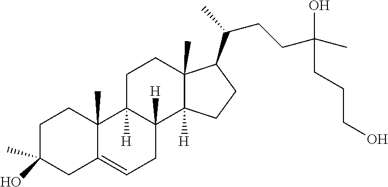 |
| B182 | 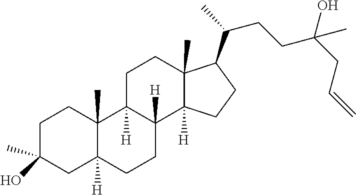 |
| B183 | 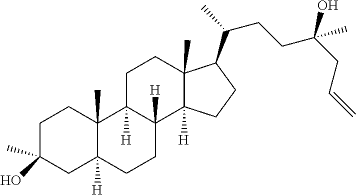 |
| B184 | 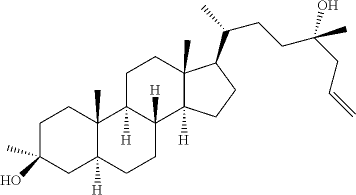 |
| B185 | 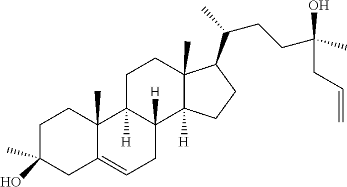 |
| B186 | 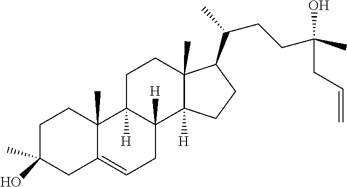 |
| B187 | 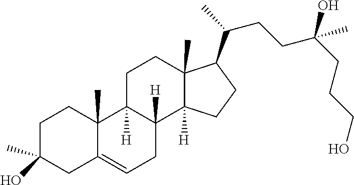 |
| B188 | 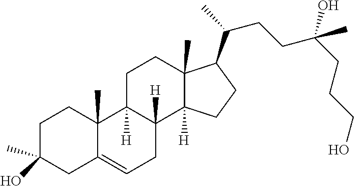 |
| B189 | 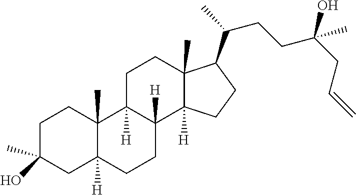 |
| B190 | 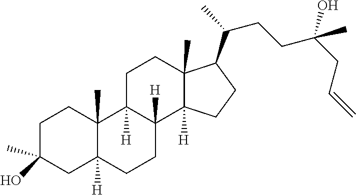 |
| B191 | 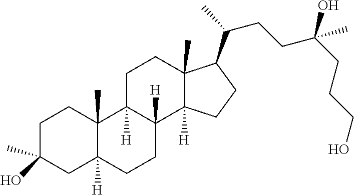 |
| B192 | 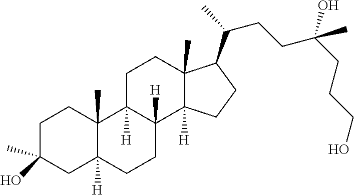 |
| B193 | 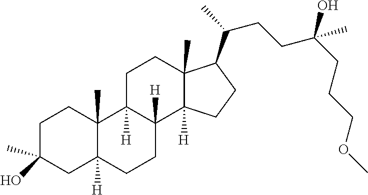 |
| B194 | 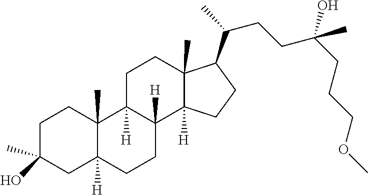 |
| B195 | 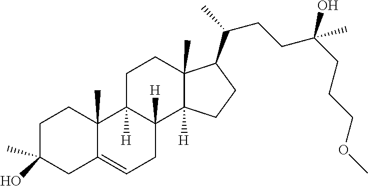 |
| B196 | 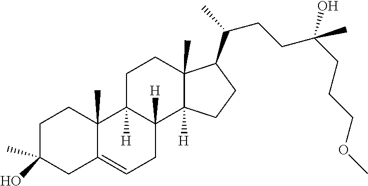 |
| B197 | 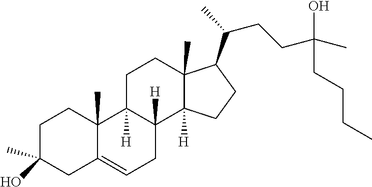 |
| B198 | 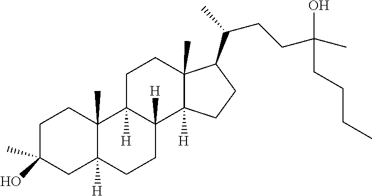 |
| B199 | 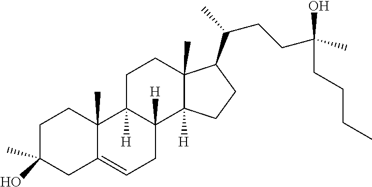 |
| B200 | 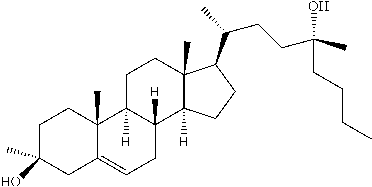 |
| B201 | 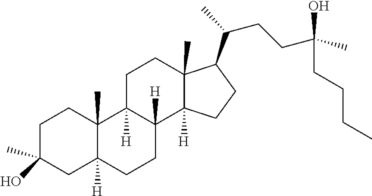 |
| B202 | 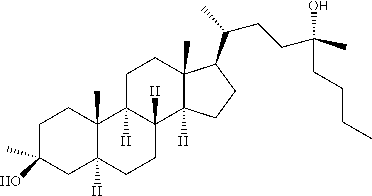 |
| B203 | 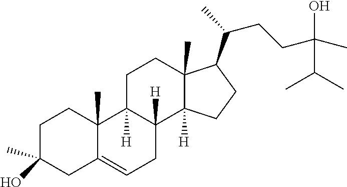 |
| B204 | 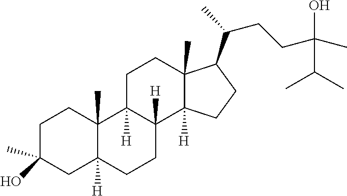 |
| B205 | 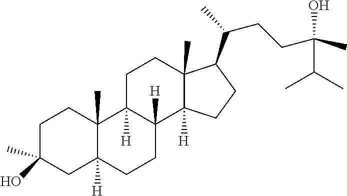 |
| B206 | 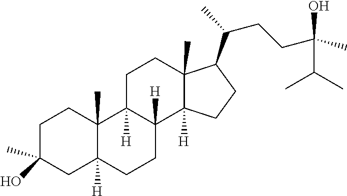 |
| B207 | 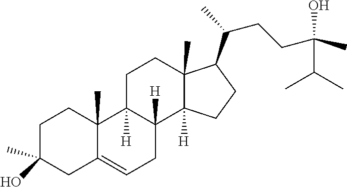 |
| B208 | 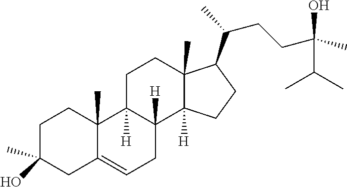 |
| B209 | 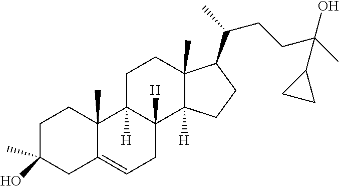 |
| B210 | 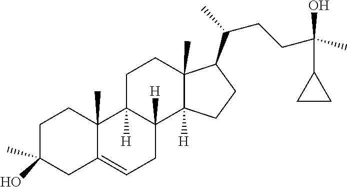 |
| B211 | 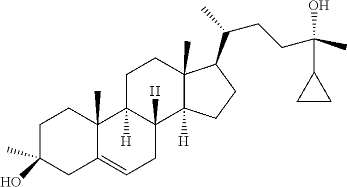 |
| B212 | 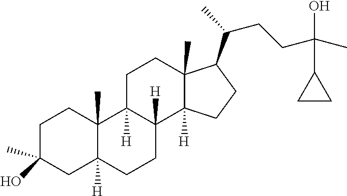 |
| B213 | 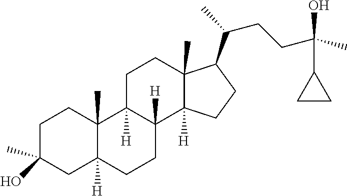 |
| B214 | 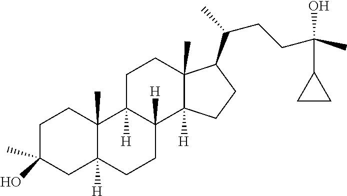 |
| B215 | 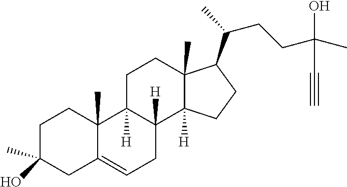 |
| B216 | 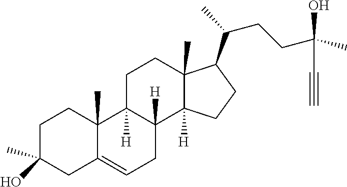 |
| B217 | 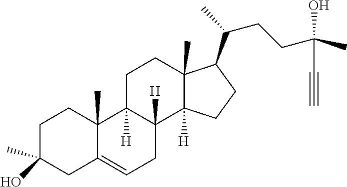 |
| B218 | 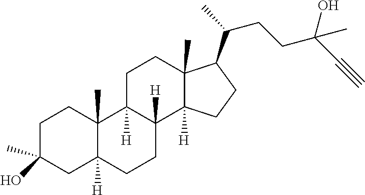 |
| B219 | 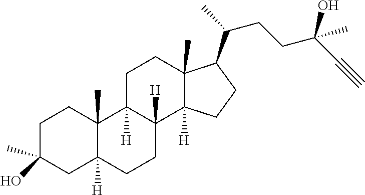 |
| B220 | 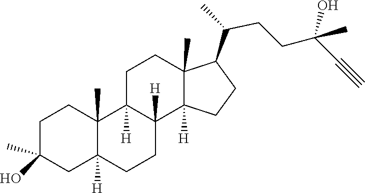 |
| B221 | 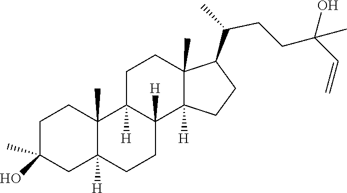 |
| B222 | 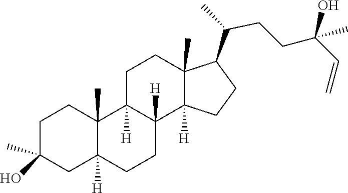 |
| B223 | 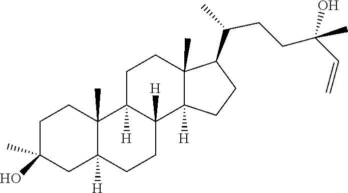 |
| B224 | 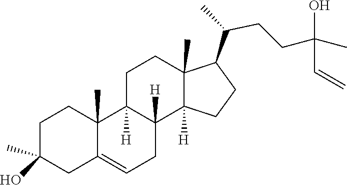 |
| B225 | 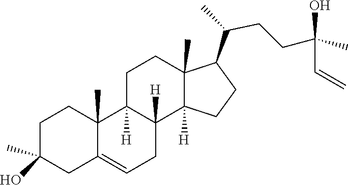 |
| B226 | 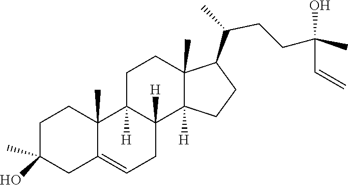 |
| B227 | 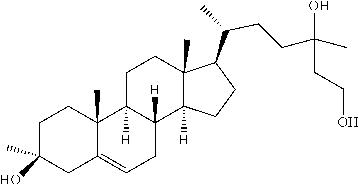 |
| B228 | 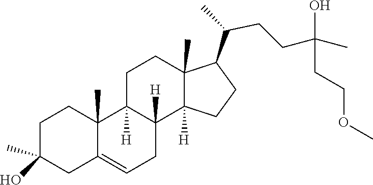 |
| B229 | 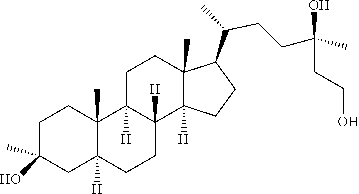 |
| B230 | 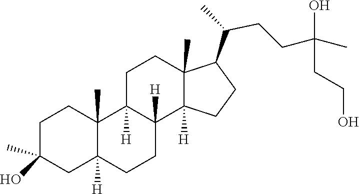 |
| B231 | 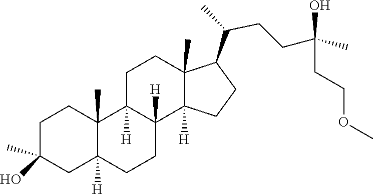 |
| B232 | 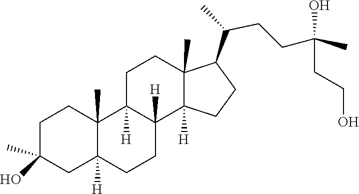 |
| B233 | 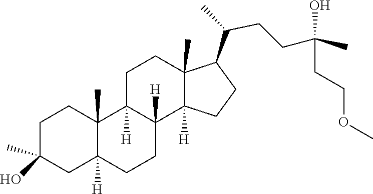 |
| B234 | 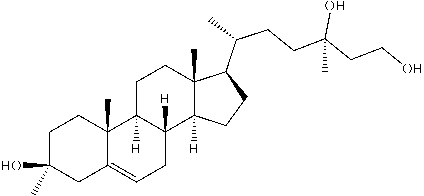 |
| B235 | 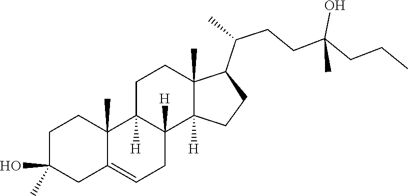 |
| B236 | 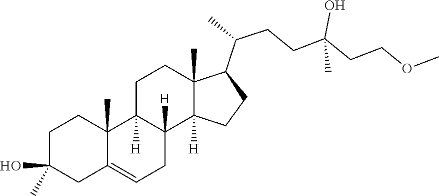 |
| B237 | 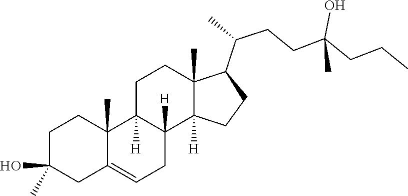 |
| B238 | 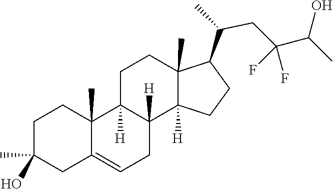 |
| B239 | 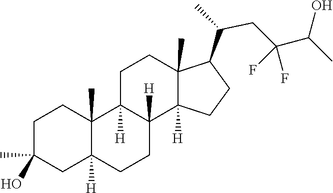 |
| B240 | 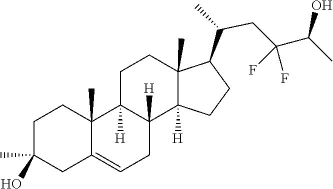 |
| B241 | 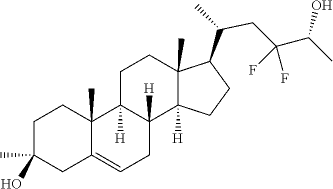 |
| B242 | 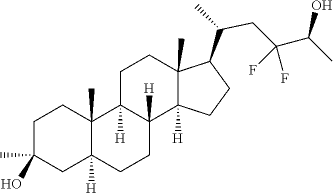 |
| B243 | 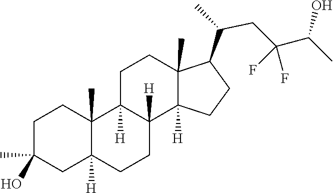 |
| B244 | 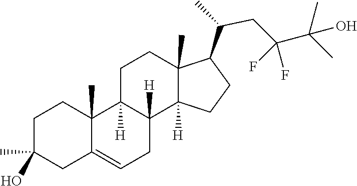 |
| B245 | 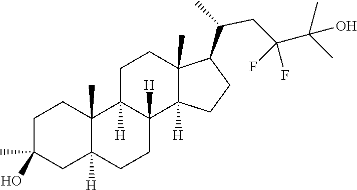 |
| B246 | 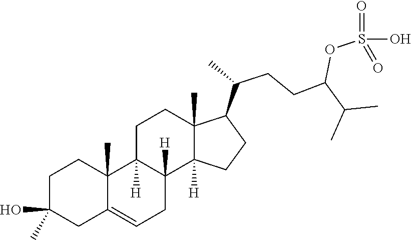 |
| B247 | 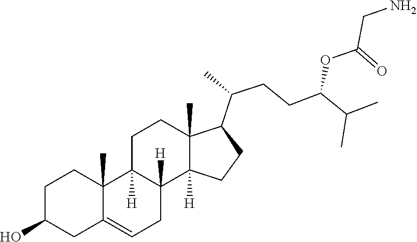 |
| B248 | 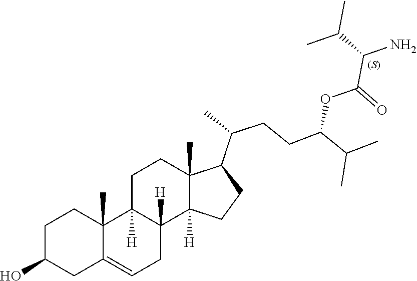 |
| B249 | 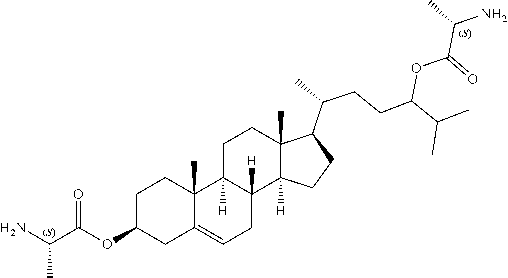 |
| B250 | 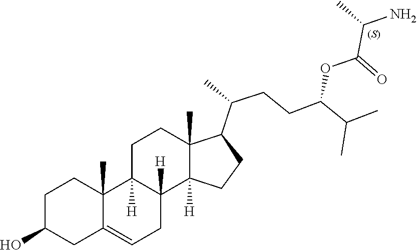 |
| B251 | 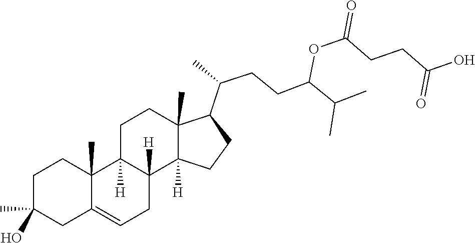 |
| B252 | 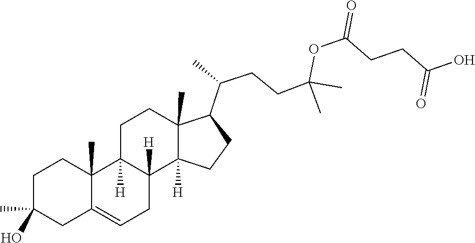 |
| B253 | 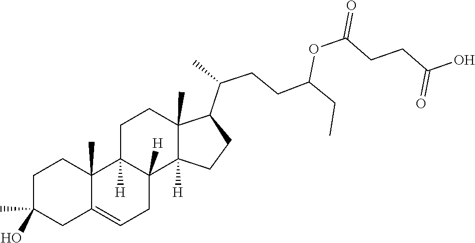 |
| B254 | 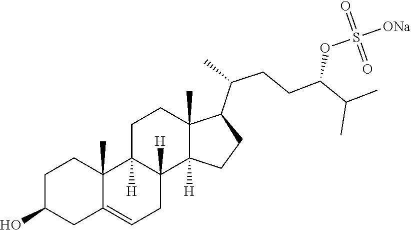 |
| B255 | 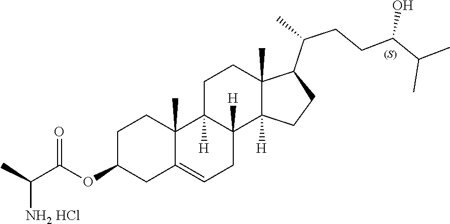 |
| B256 | 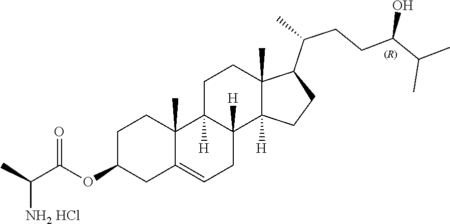 |
| B257 | 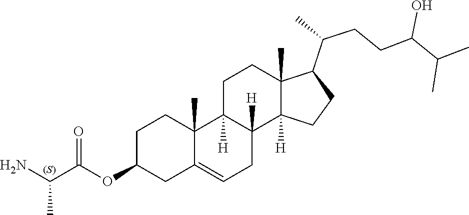 |
| B258 | 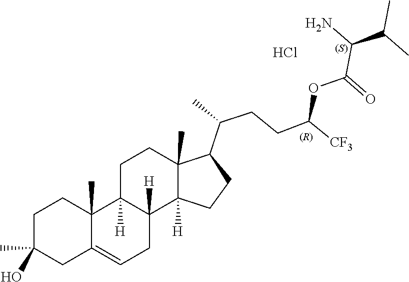 |
| B259 | 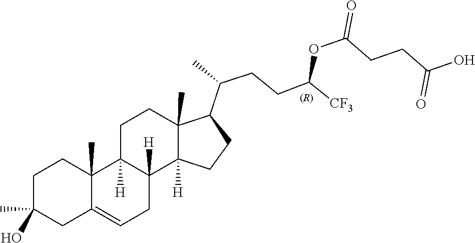 |
| B260 | 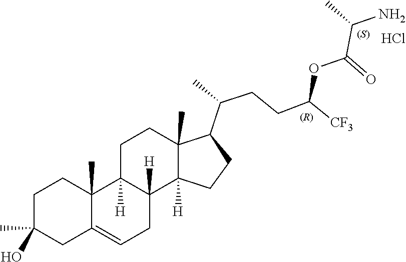 |
| B261 | 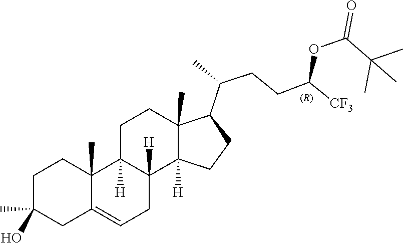 |
| B262 | 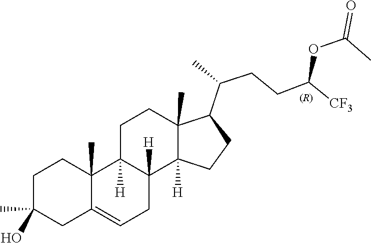 |
| B263 | 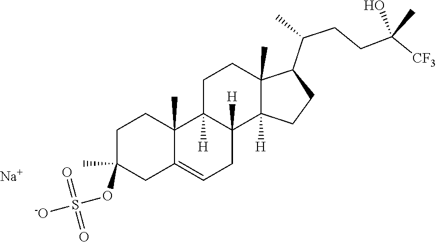 |
| B264 | 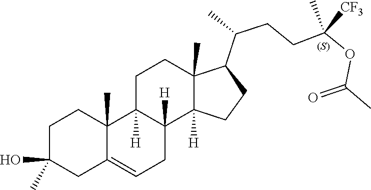 |
| B265 | 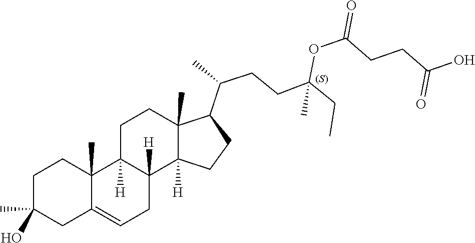 |
| B266 | 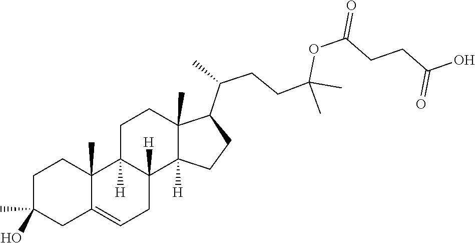 |
| B267 | 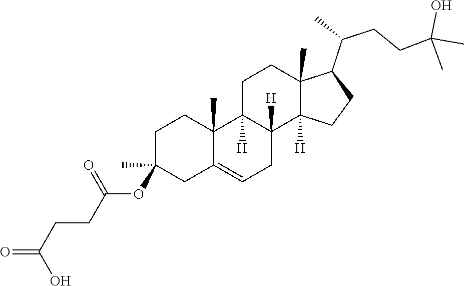 |
| B268 | 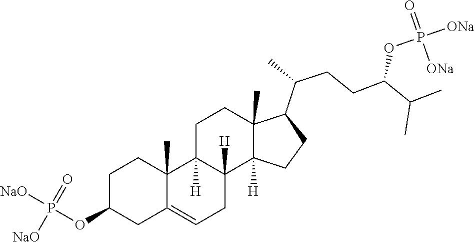 |
| B269 | 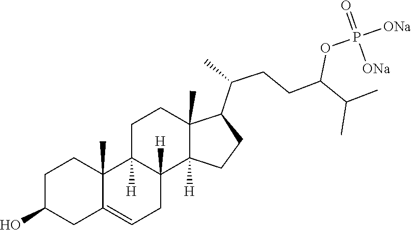 |
| B270 | 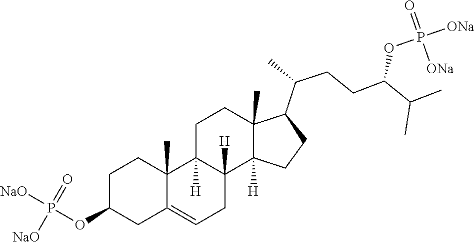 |
| B271 | 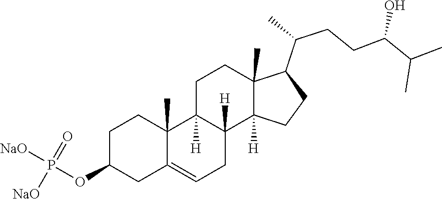 |
| B272 | 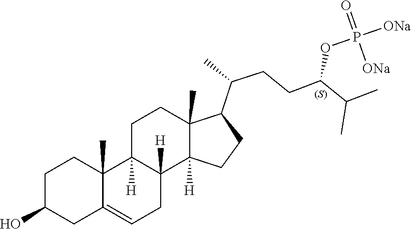 |
| B273 | 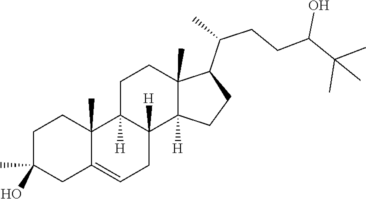 |
| B274 | 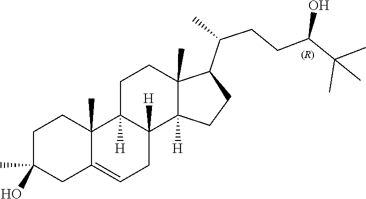 |
| B275 | 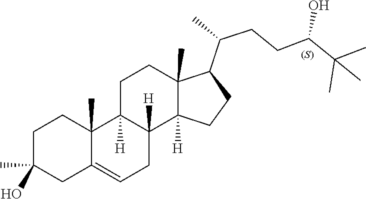 |
| B276 | 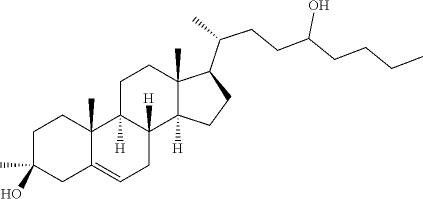 |
| B277 | 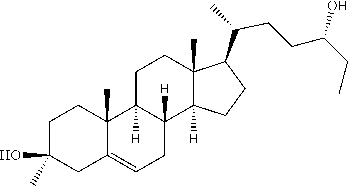 |
| B278 | 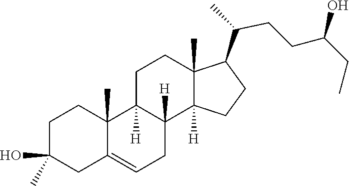 |
| B279 | 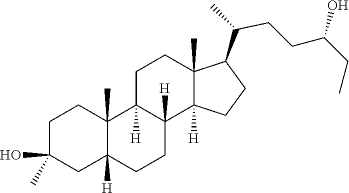 |
| B280 | 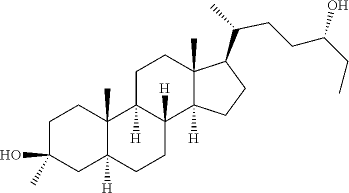 |
| B281 | 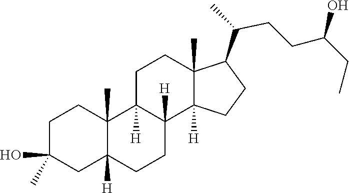 |
| B282 | 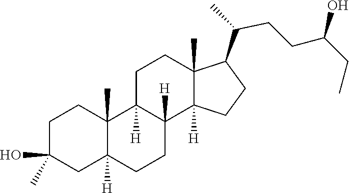 |
| B283 | 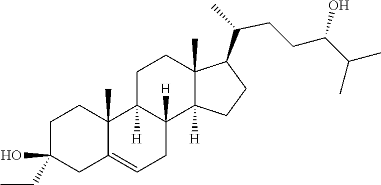 |
| B284 | 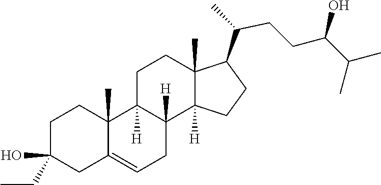 |
| B285 | 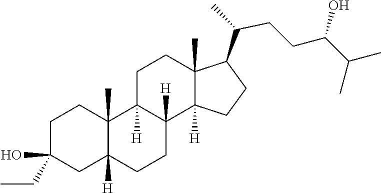 |
| B286 | 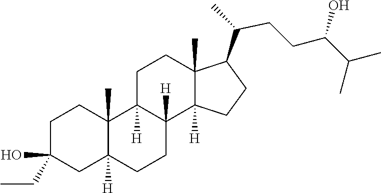 |
| B287 | 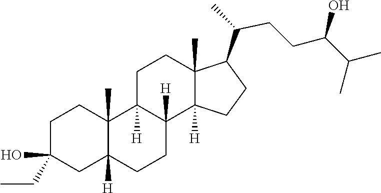 |
| B288 | 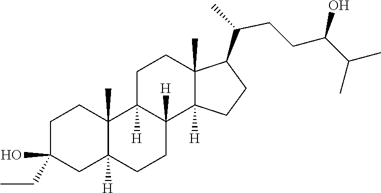 |
| B289 | 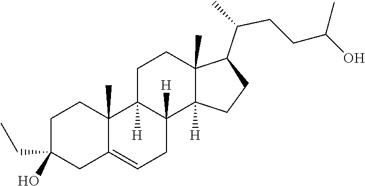 |
| B290 | 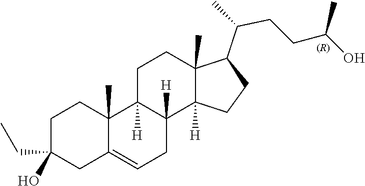 |
| B291 | 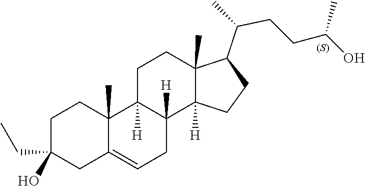 |
| B292 | 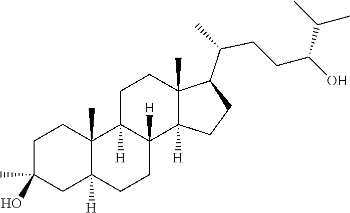 |
| B293 | 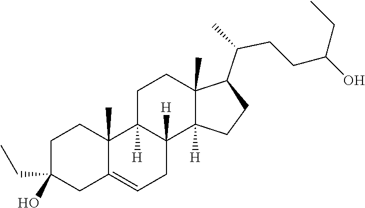 |
| B294 | 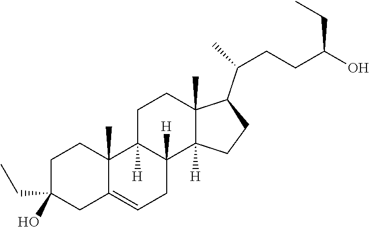 |
| B295 | 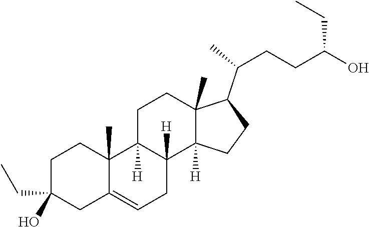 |
| B296 | 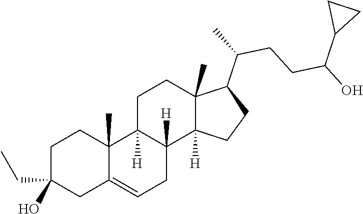 |
| B297 | 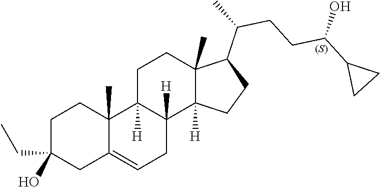 |
| B298 | 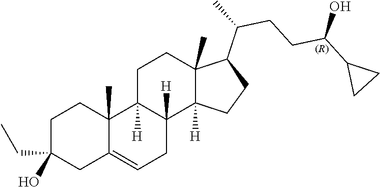 |
| B299 | 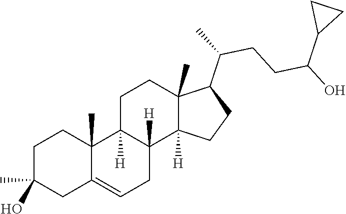 |
| B300 | 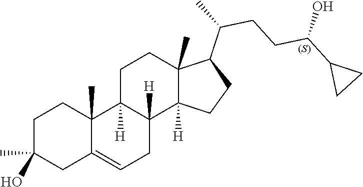 |
| B301 | 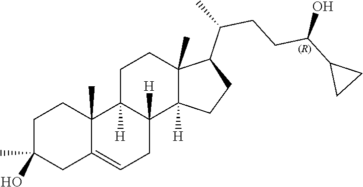 |
| B302 | 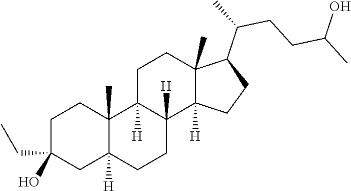 |
| B303 | 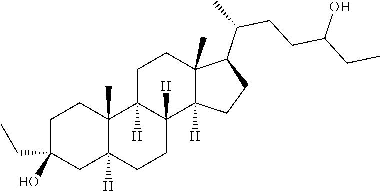 |
| B304 | 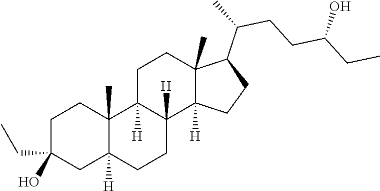 |
| B305 | 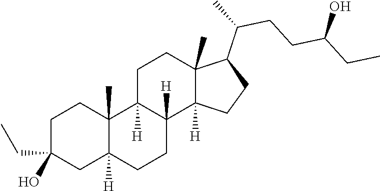 |
| B306 | 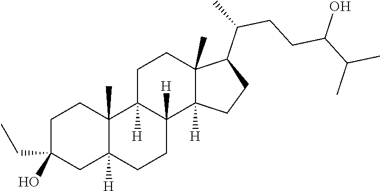 |
| B307 | 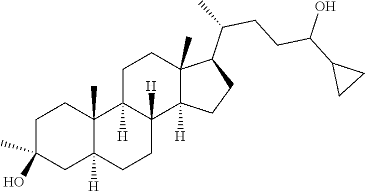 |
| B308 | 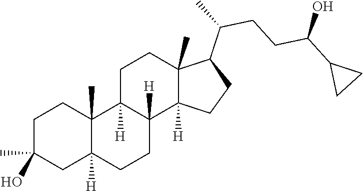 |
| B309 | 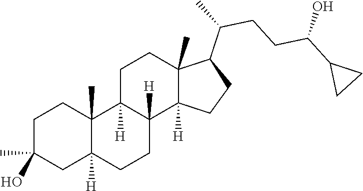 |
| B310 | 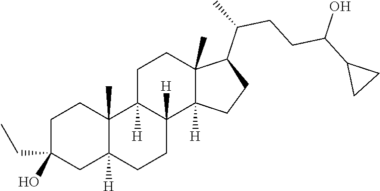 |
| B311 | 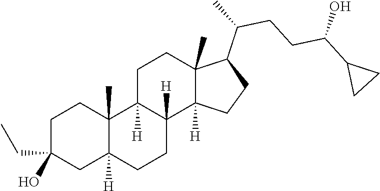 |
| B312 | 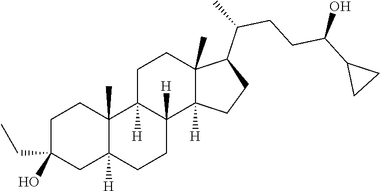 |
| B313 | 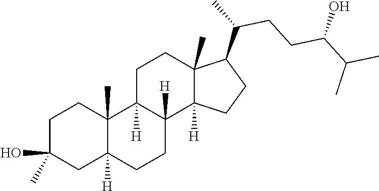 |
| B314 | 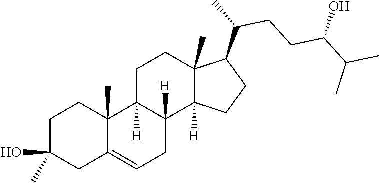 |
| B315 | 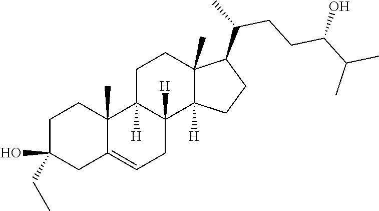 |
| B316 | 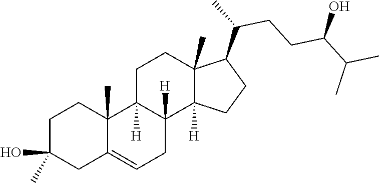 |
| B317 | 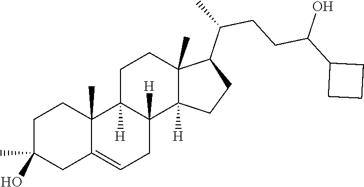 |
| B318 | 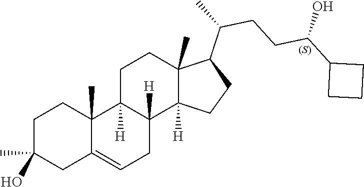 |
| B319 | 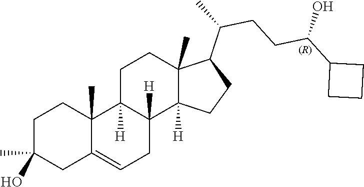 |
| B320 | 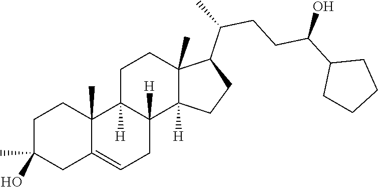 |
| B321 | 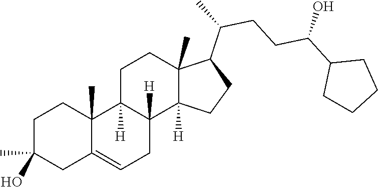 |
| B322 | 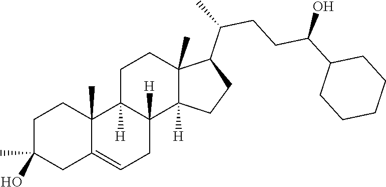 |
| B323 | 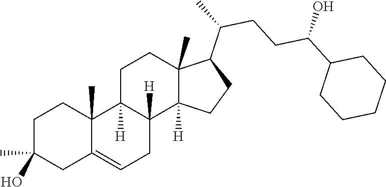 |
| B324 | 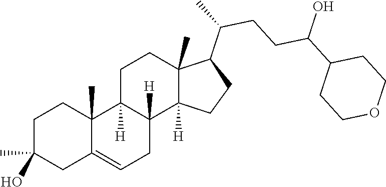 |
| B325 | 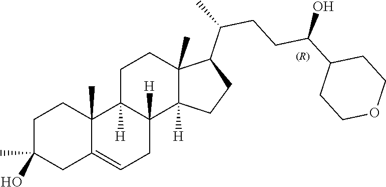 |
| B326 | 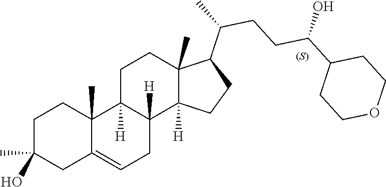 |
| B327 | 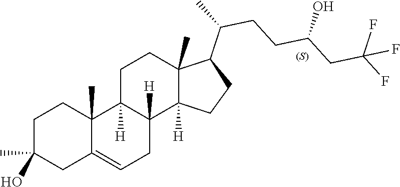 |
| B328 | 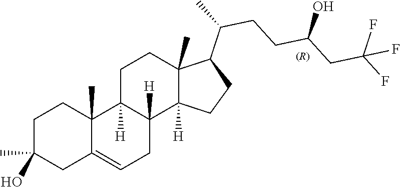 |
| B329 | 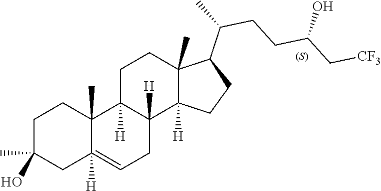 |
| B330 | 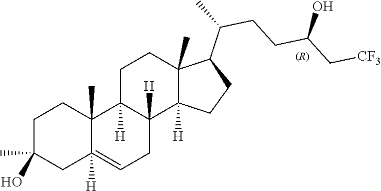 |
| B331 | 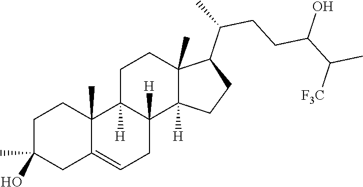 |
| B332 | 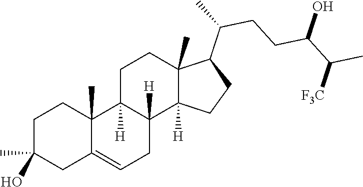 |
| B333 | 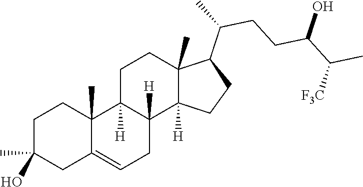 |
| B334 | 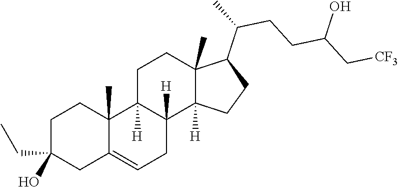 |
| B335 | 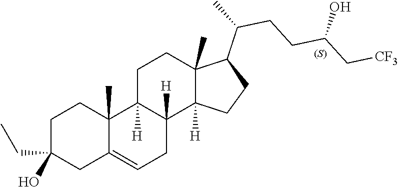 |
| B336 | 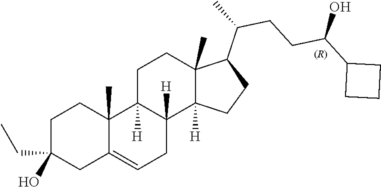 |
| B337 | 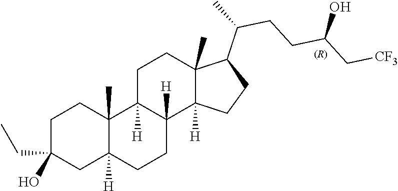 |
| B338 | 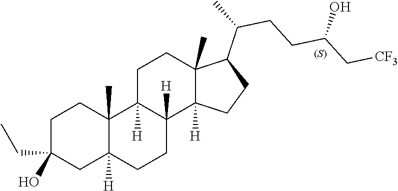 |
| B339 | 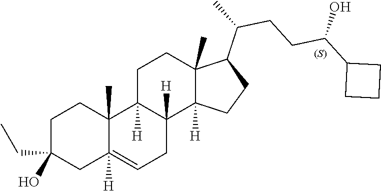 |
| B340 | 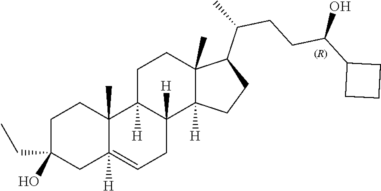 |
| B341 | 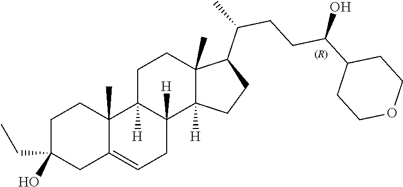 |
| B342 | 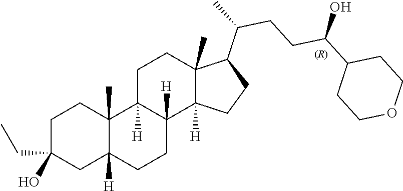 |
| B343 | 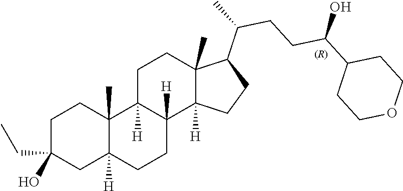 |
| B344 | 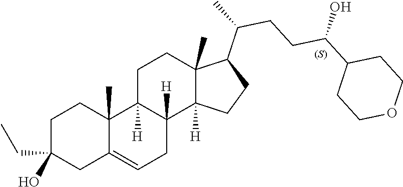 |
| B345 | 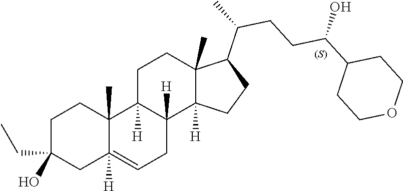 |
| B346 | 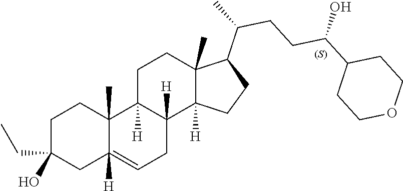 |
| B347 | 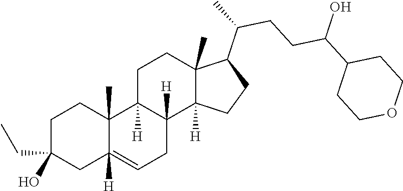 |
| B348 | 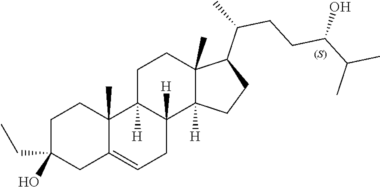 |
| B349 | 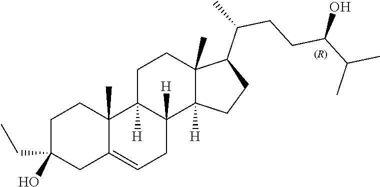 |
| B350 | 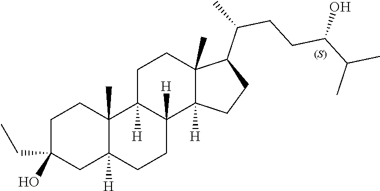 |
| B351 | 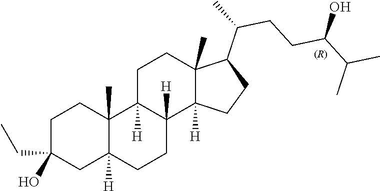 |
| B352 | 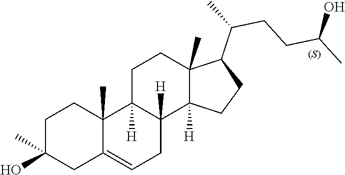 |
| B353 | 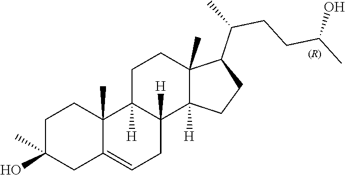 |
| B354 | 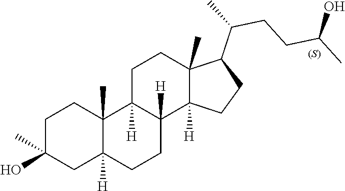 |
| B355 | 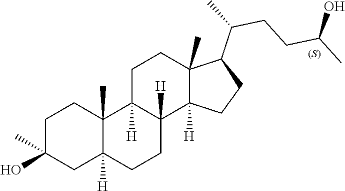 |
| B356 | 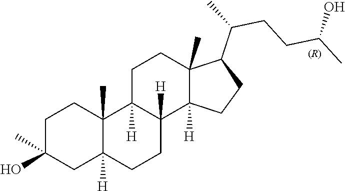 |
| B357 | 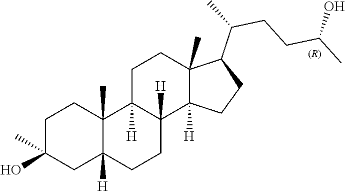 |
| B358 | 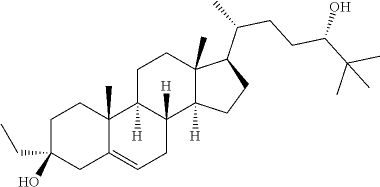 |
| B359 | 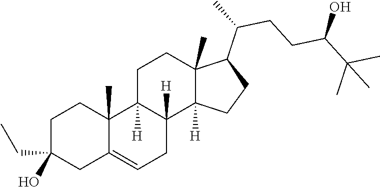 |
| B360 | 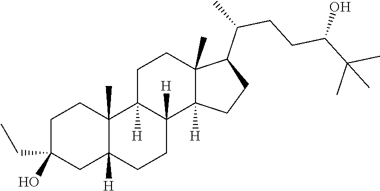 |
| B361 | 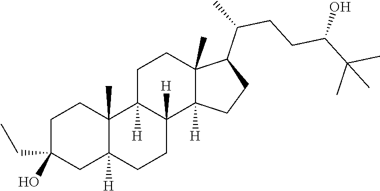 |
| B362 | 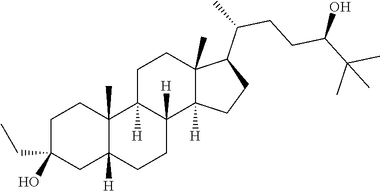 |
| B363 | 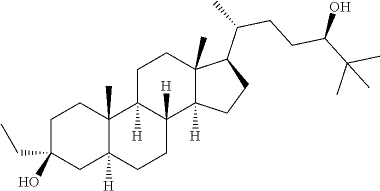 |
| B364 | 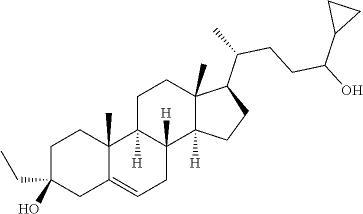 |
| B365 | 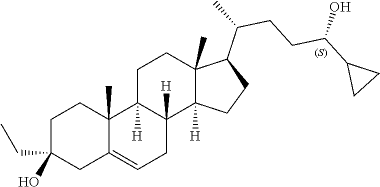 |
| B366 | 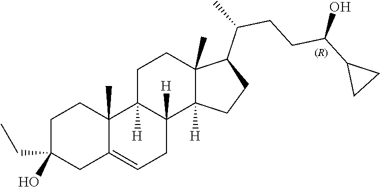 |
| B367 | 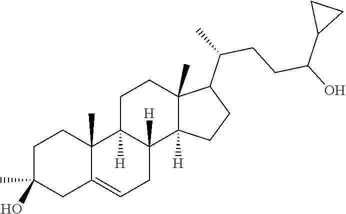 |
| B368 | 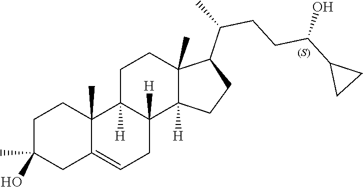 |
| B369 | 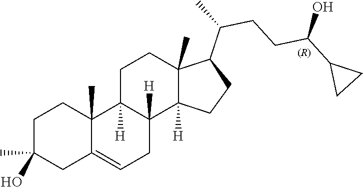 |
| B370 | 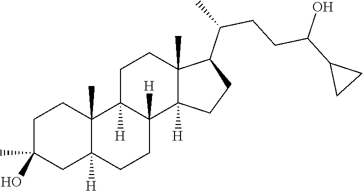 |
| B371 | 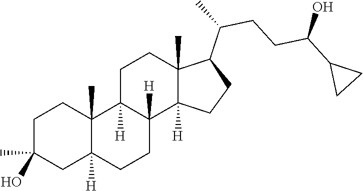 |
| B372 | 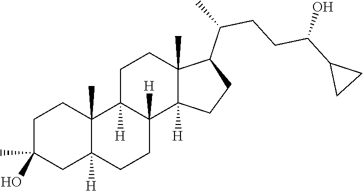 |
| B373 | 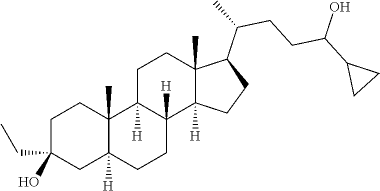 |
| B374 | 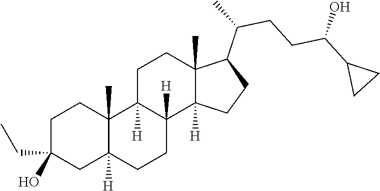 |
| B375 | 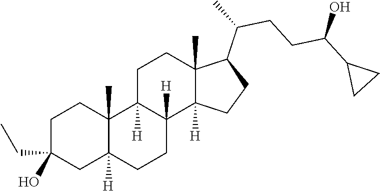 |
| B376 | 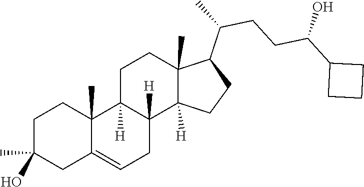 |
| B377 | 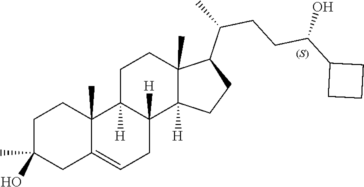 |
| B378 | 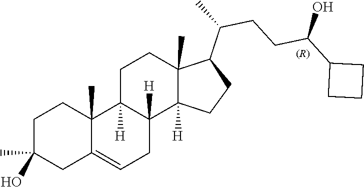 |
| B379 | 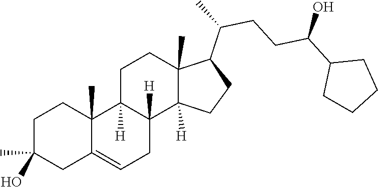 |
| B380 | 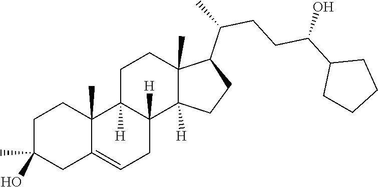 |
| B381 | 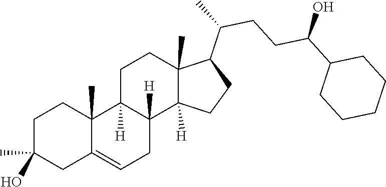 |
| B382 | 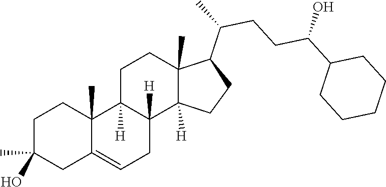 |
| B383 | 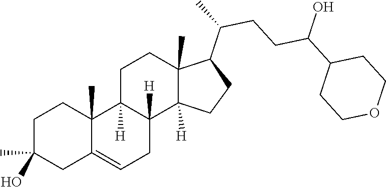 |
| B384 | 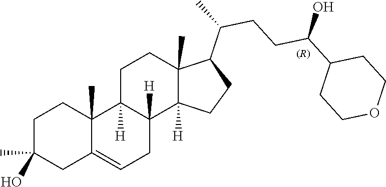 |
| B385 | 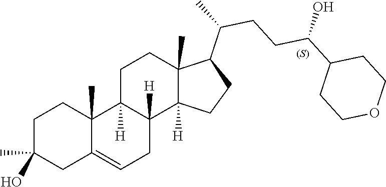 |
| B386 | 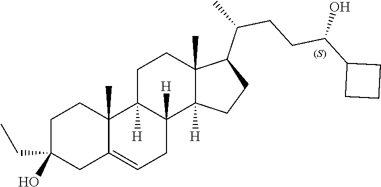 |
| B387 | 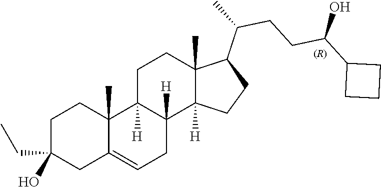 |
| B388 | 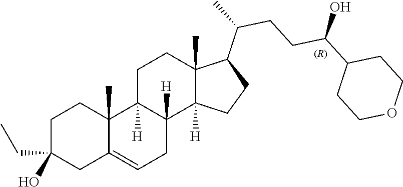 |
| B389 | 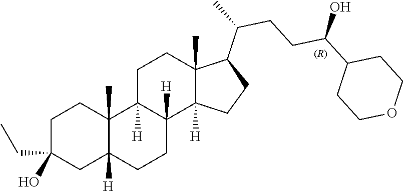 |
| B390 | 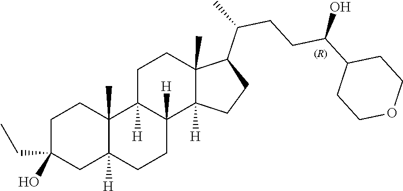 |
| B391 | 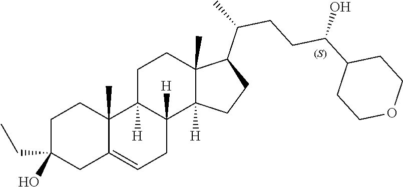 |
| B392 | 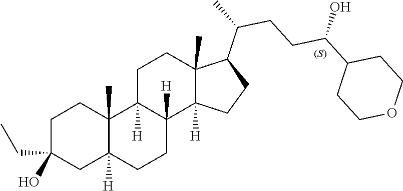 |
| B393 | 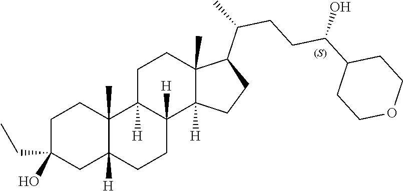 |
| B394 | 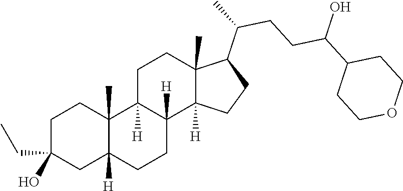 |
| B395 | 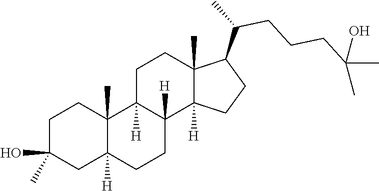 |
| B396 | 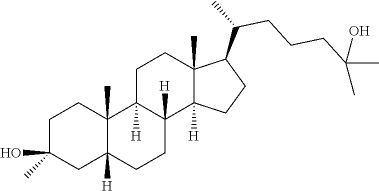 |
| B397 | 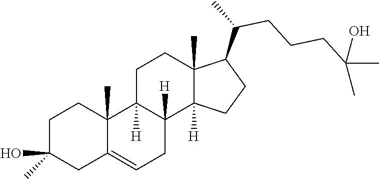 |
| B398 | 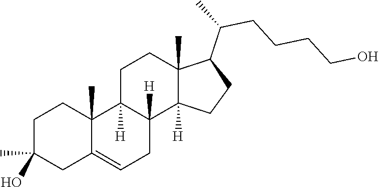 |
| B399 | 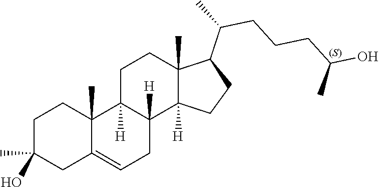 |
| B400 | 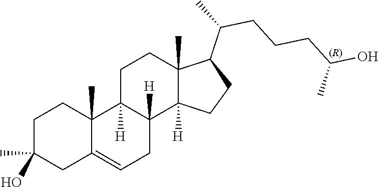 |
| B401 | 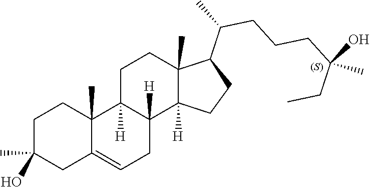 |
| B402 | 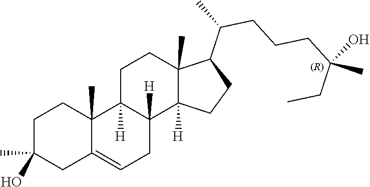 |
| B403 | 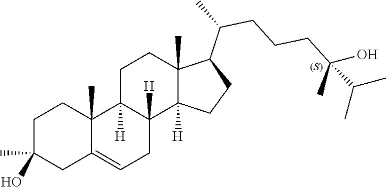 |
| B404 | 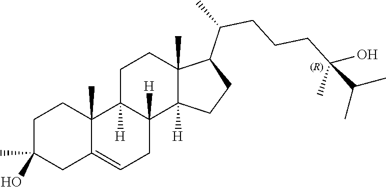 |
| B405 | 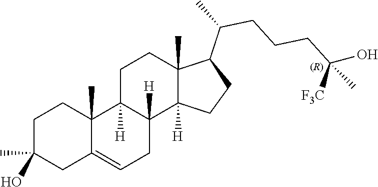 |
| B405 | 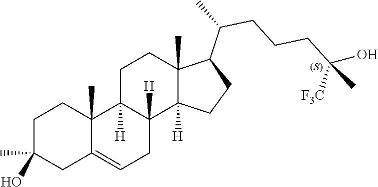 |
| B407 | 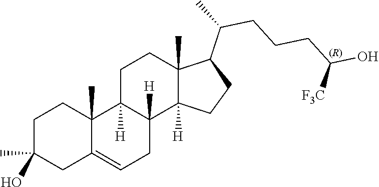 |
| B408 | 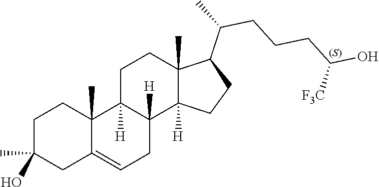 |
| B409 | 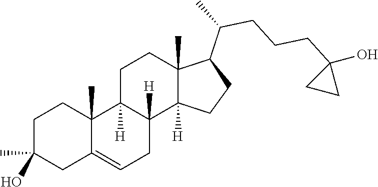 |
| B410 | 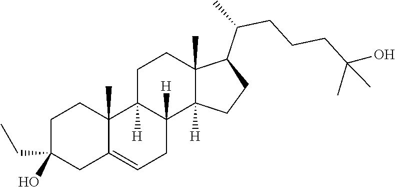 |
| B411 | 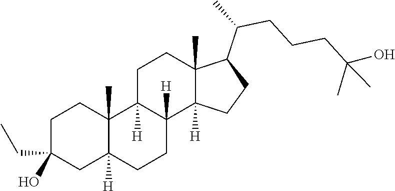 |
| B412 | 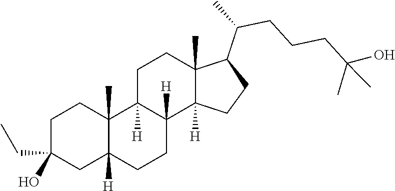 |
| B413 | 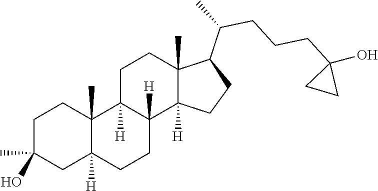 |
| B414 | 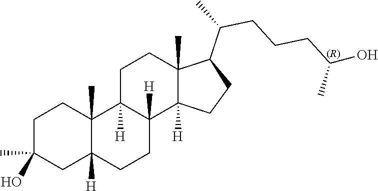 |
| B415 | 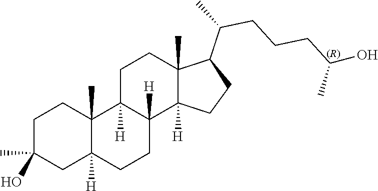 |
| B416 | 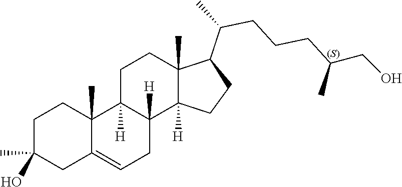 |
| B417 | 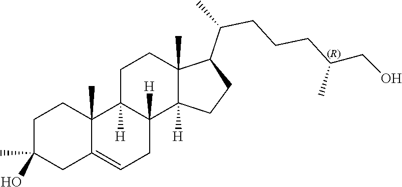 |
| B418 | 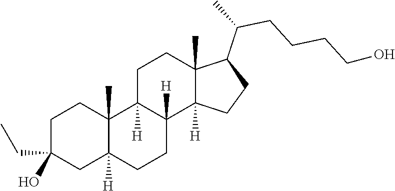 |
| B419 | 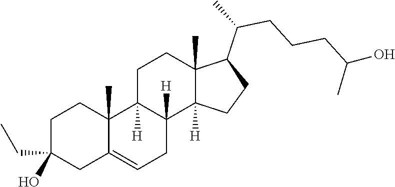 |
| B420 | 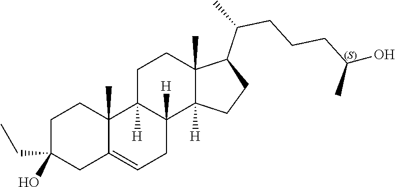 |
| B421 | 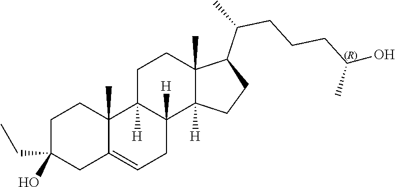 |
| B422 | 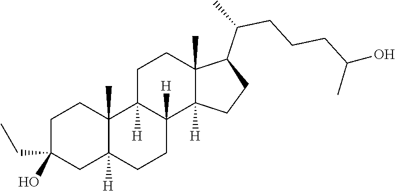 |
| B423 | 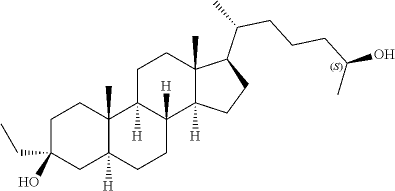 |
| B424 | 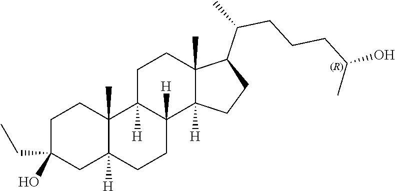 |
| B425 | 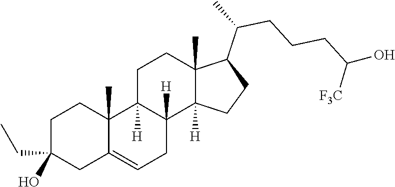 |
| B426 | 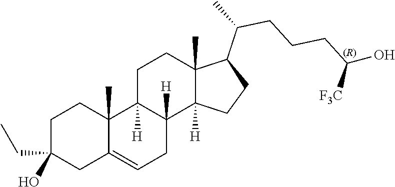 |
| B427 | 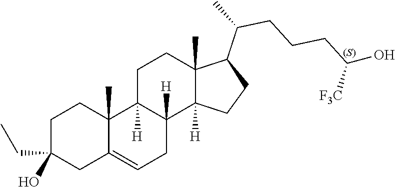 |
| B428 | 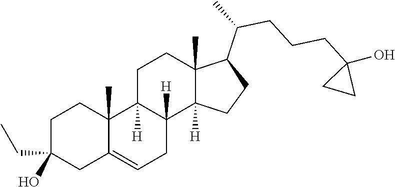 |
| B429 | 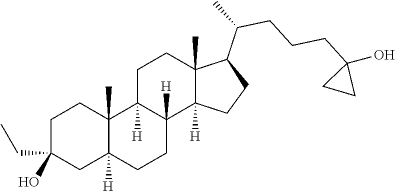 |
| B430 | 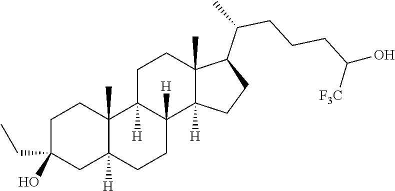 |
| B431 | 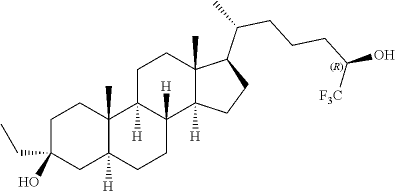 |
| B432 | 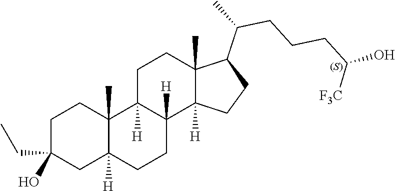 |
| B433 | 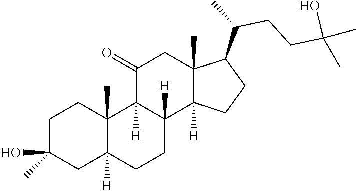 |
| B434 | 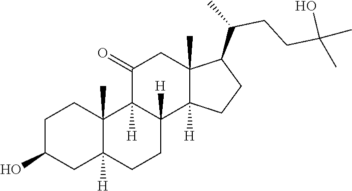 |
| B435 | 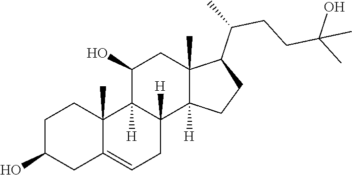 |
| B436 | 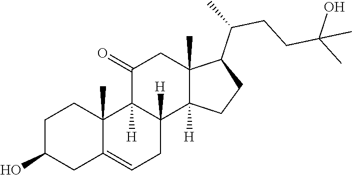 |
| B437 | 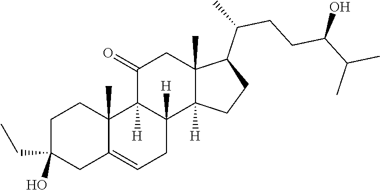 |
| B438 | 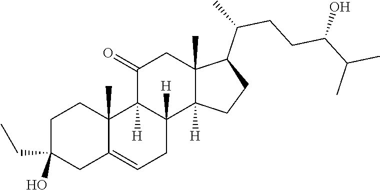 |
| B439 | 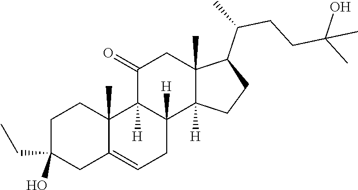 |
| B440 | 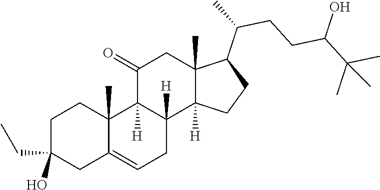 |
| B441 | 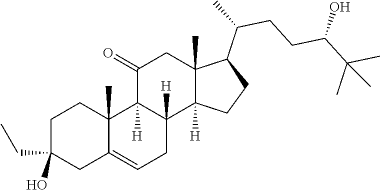 |
| B442 | 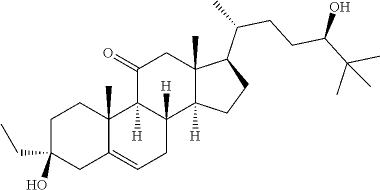 |
| B443 | 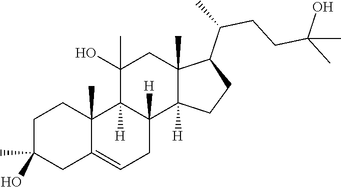 |
| B444 | 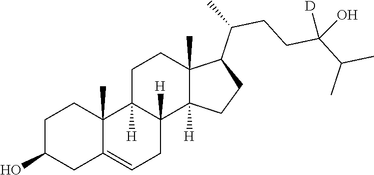 |
| B445 | 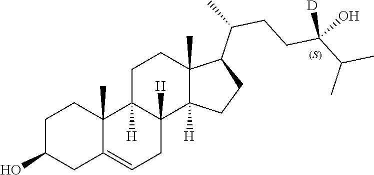 |
| B446 | 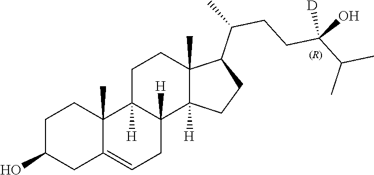 |
| B447 | 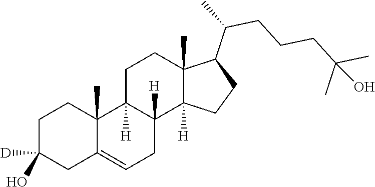 |
| B448 | 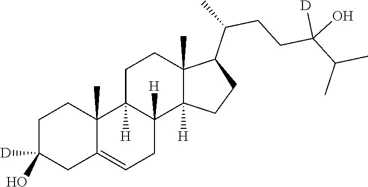 |
| B449 | 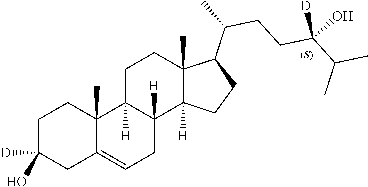 |
| B450 | 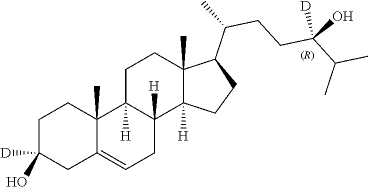 |
| B451 | 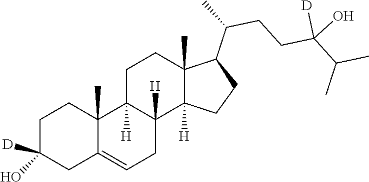 |
| B452 | 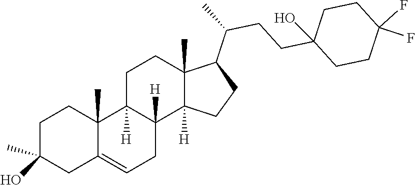 |
| B453 | 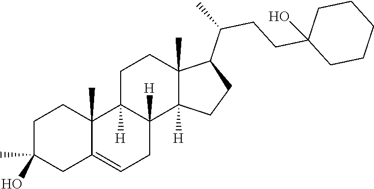 |
| B454 | 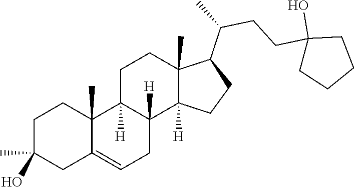 |
| B455 | 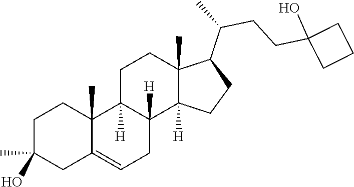 |
| B456 | 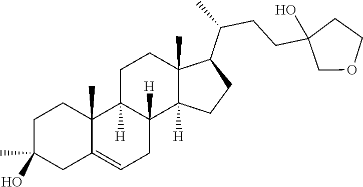 |
| B457 | 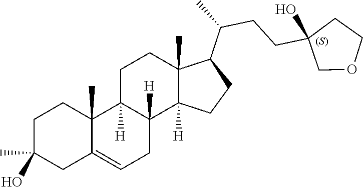 |
| B458 | 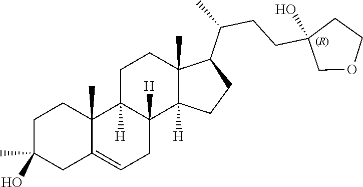 |
| B459 | 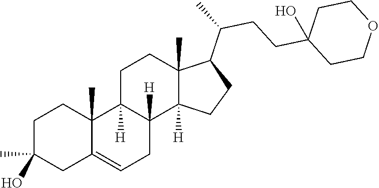 |
| B450 | 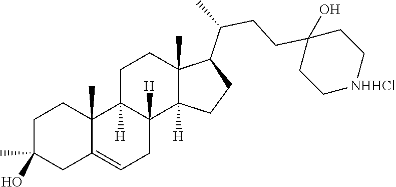 |
| B451 | 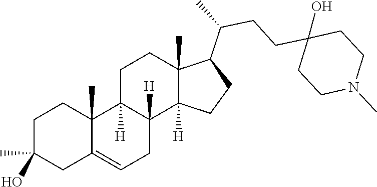 |
| B452 | 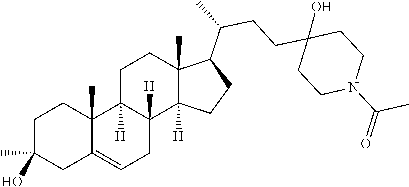 |
| B453 | 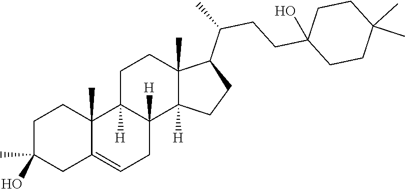 |
| B454 | 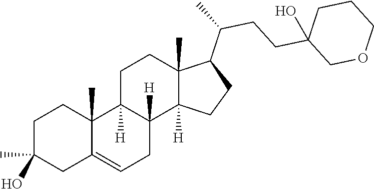 |
| B455 | 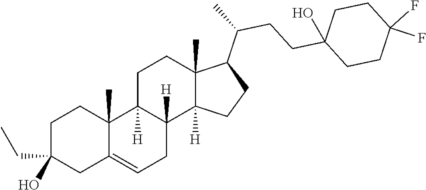 |
| B456 | 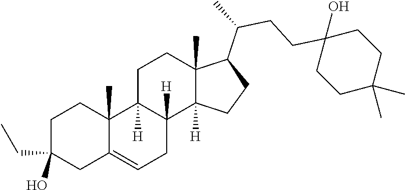 |
| B457 | 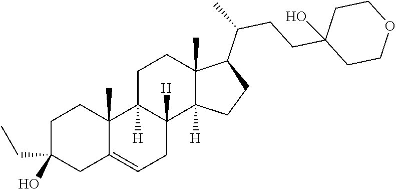 |
| B458 | 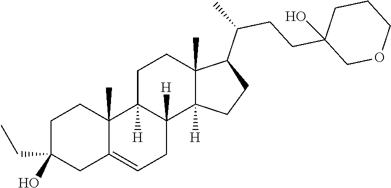 |
| B459 | 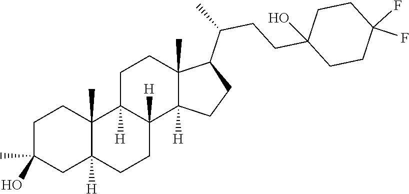 |
| B460 | 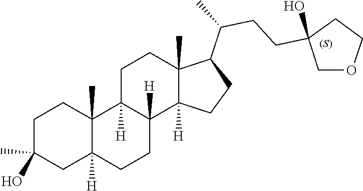 |
| B461 | 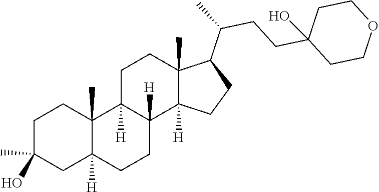 |
| B462 | 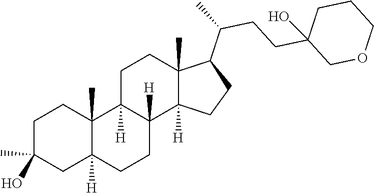 |
| B463 | 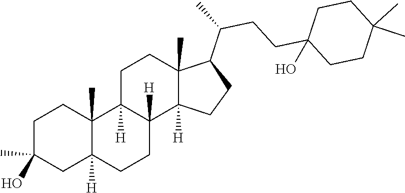 |
| B464 | 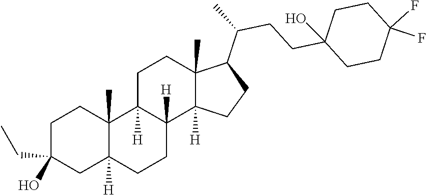 |
| B465 | 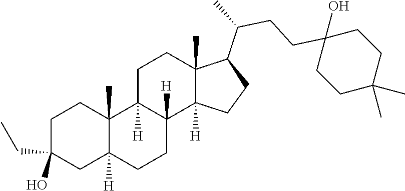 |
| B466 | 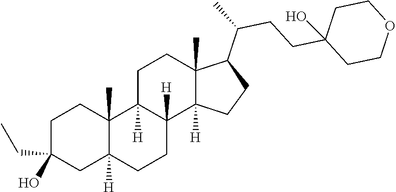 |
| B467 | 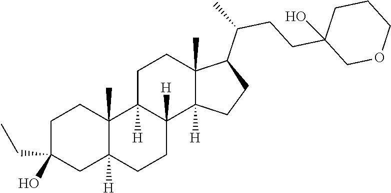 |
| B468 | 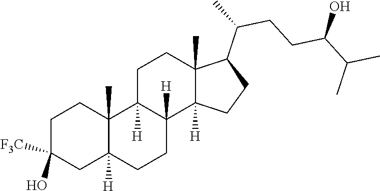 |
| B469 | 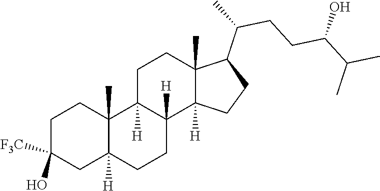 |
| B470 | 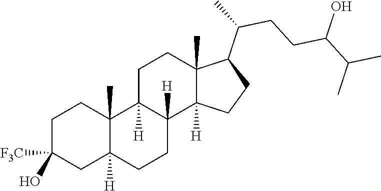 |
| B471 | 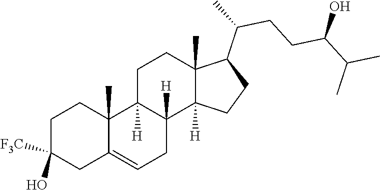 |
| B472 | 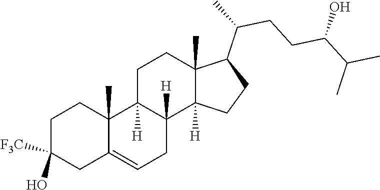 |
| B473 | 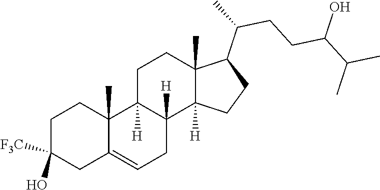 |
| B474 | 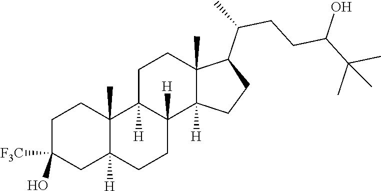 |
| B475 | 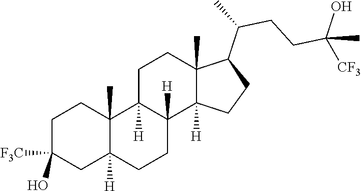 |
| B476 | 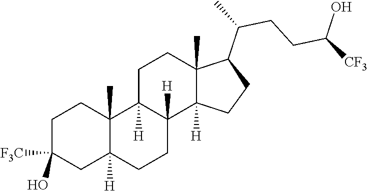 |
| B477 | 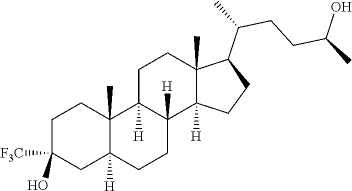 |
| B478 | 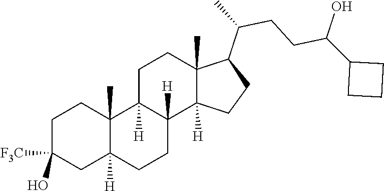 |
| B479 | 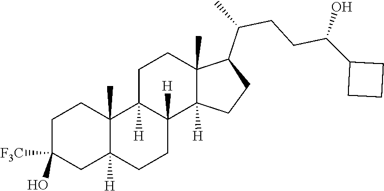 |
| B480 | 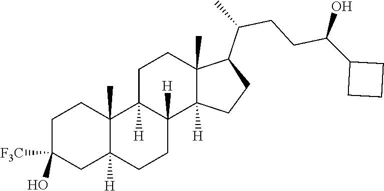 |
| B481 | 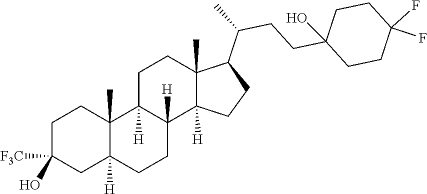 |
| B482 | 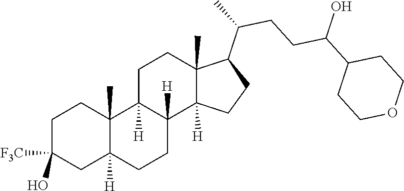 |
| B483 | 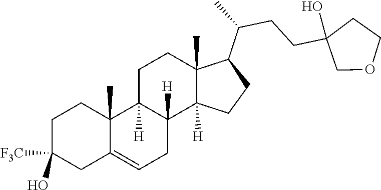 |
| B484 | 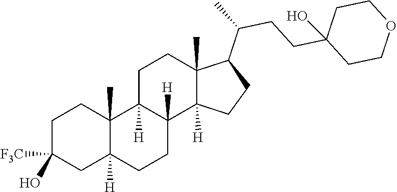 |
| B485 | 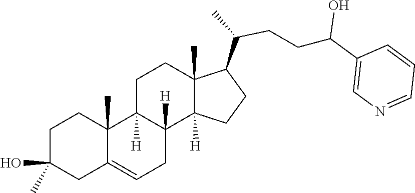 |
| B486 | 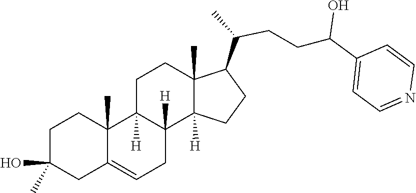 |
| B487 | 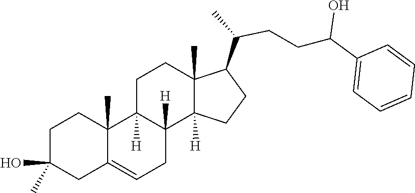 |
| B488 | 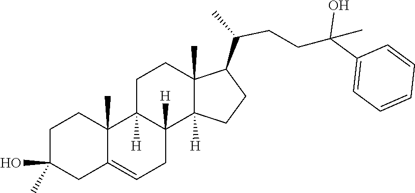 |
| B489 | 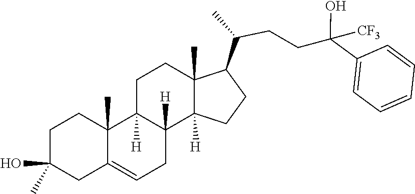 |
| B490 | 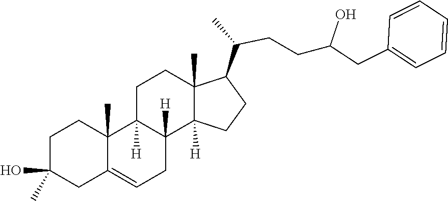 |
| B491 | 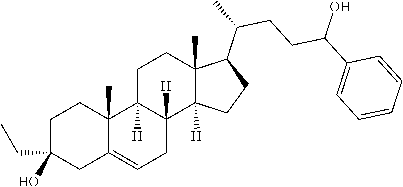 |
| B492 | 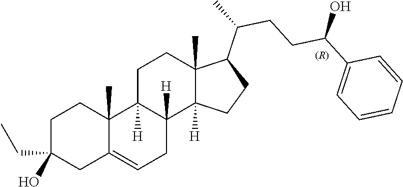 |
| B493 | 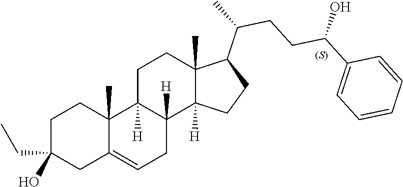 |
| B494 | 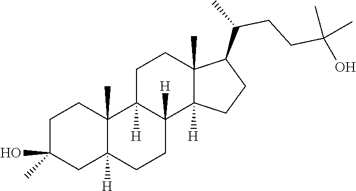 |
| B495 | 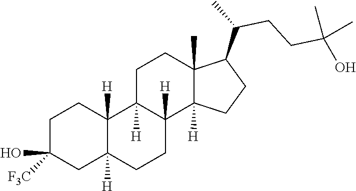 |
| B496 | 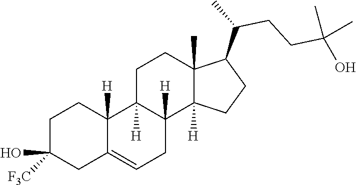 |
| B497 | 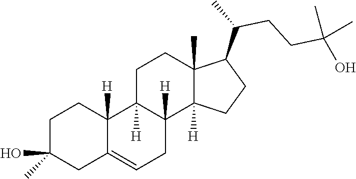 |
| B498 | 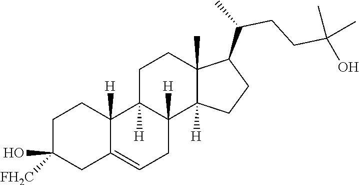 |
| B499 | 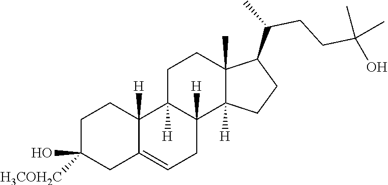 |
| B500 | 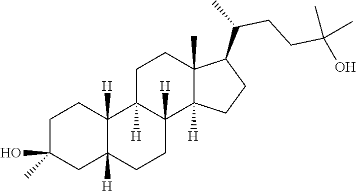 |
| B501 | 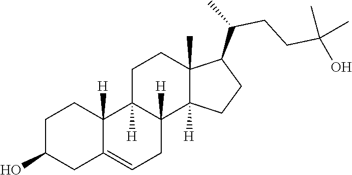 |
| B502 | 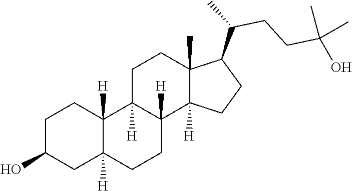 |
| B503 | 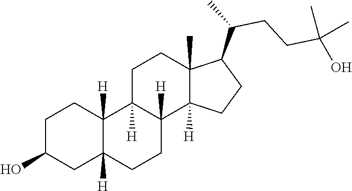 |
| B504 | 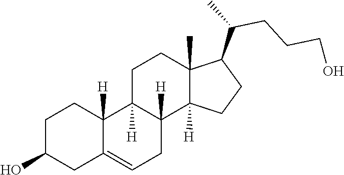 |
| B505 | 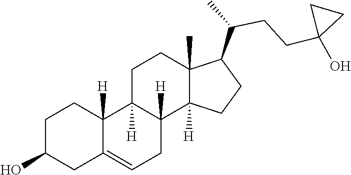 |
| B506 | 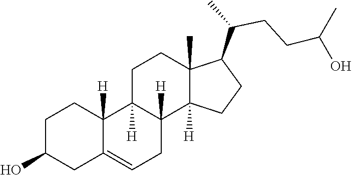 |
| B507 | 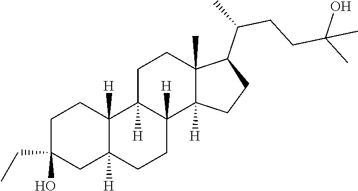 |
| B508 | 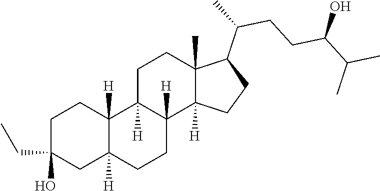 |
| B509 | 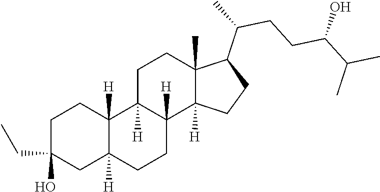 |
| B510 | 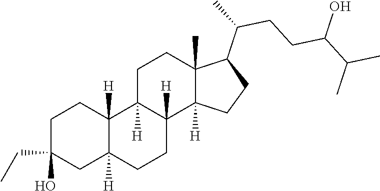 |
| B511 | 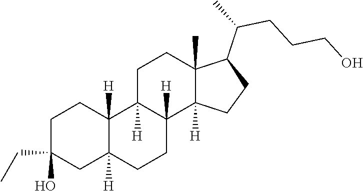 |
| B512 | 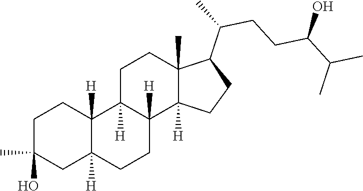 |
| B513 | 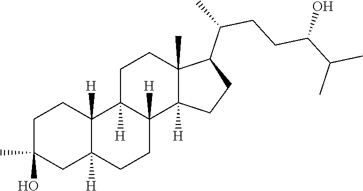 |
| B514 | 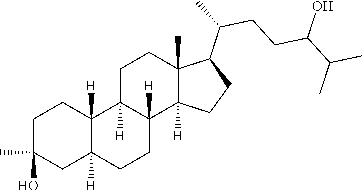 |
| B515 | 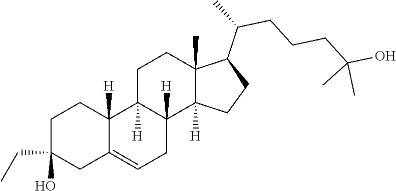 |
| B516 | 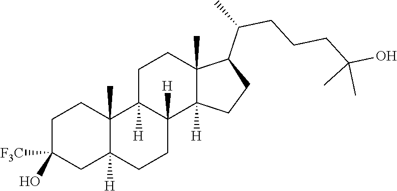 |
| B517 | 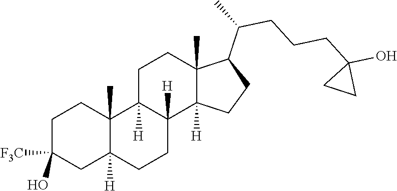 |
| B518 | 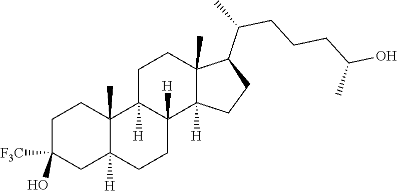 |
| B519 | 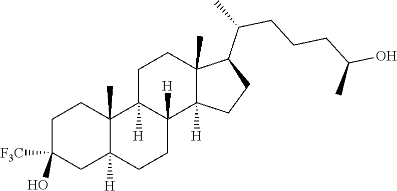 |
| B520 | 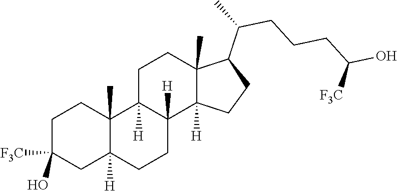 |
| B521 | 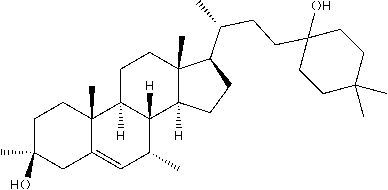 |
| B522 | 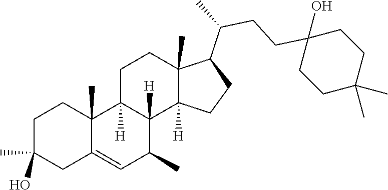 |
| B523 | 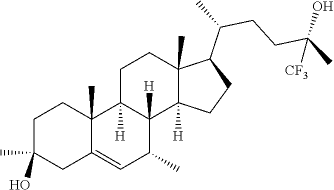 |
| B524 | 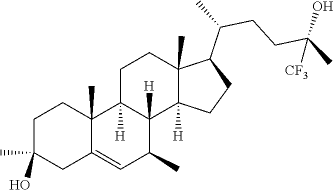 |
| B525 | 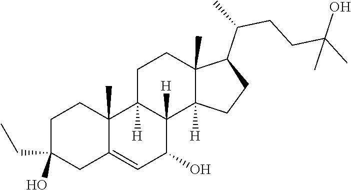 |
| B526 | 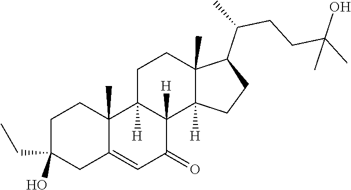 |
| B527 | 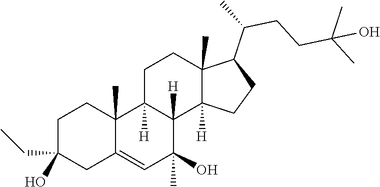 |
| B528 | 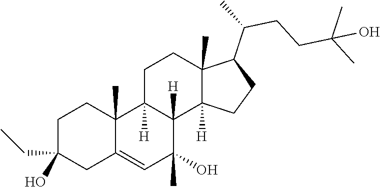 |
| B529 | 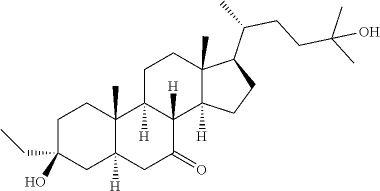 |
| B530 | 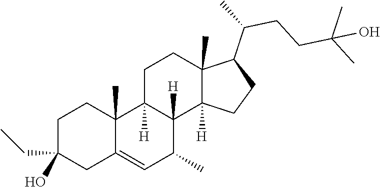 |
| B531 | 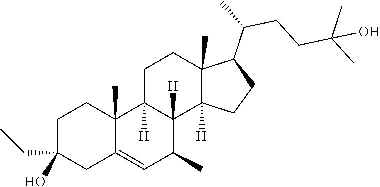 |
| B532 | 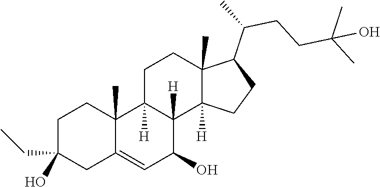 |
| B533 | 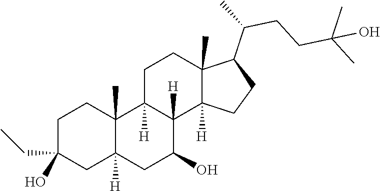 |
| B534 | 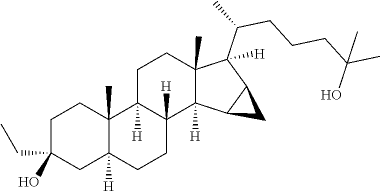 |
| B535 | 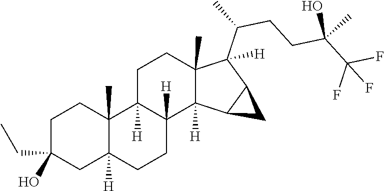 |
| B536 | 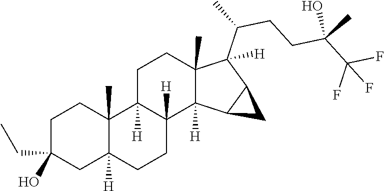 |
| B537 | 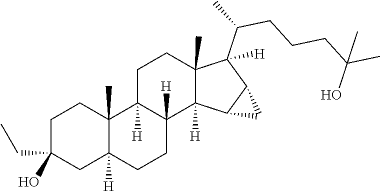 |
| B538 | 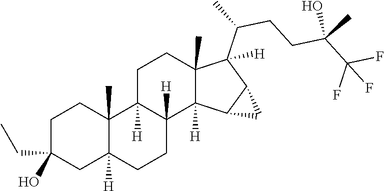 |
| B539 | 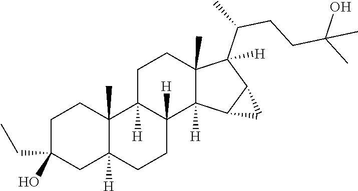 |
| B540 | 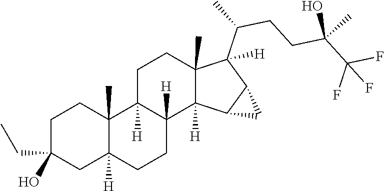 |
| B541 | 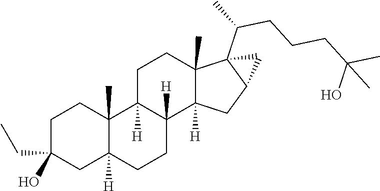 |
| B542 | 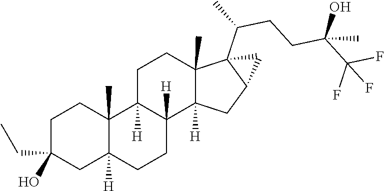 |
| B543 | 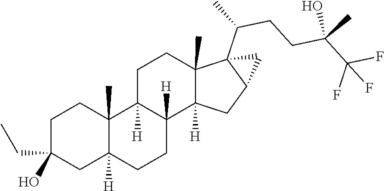 |
| B544 | 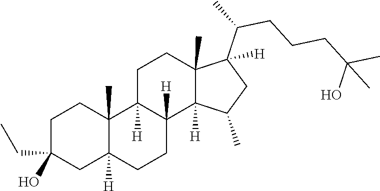 |
| B545 | 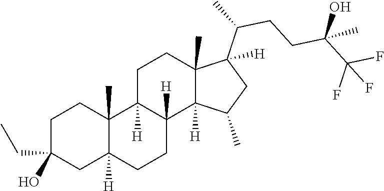 |
| B546 | 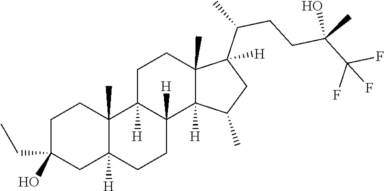 |
| B547 | 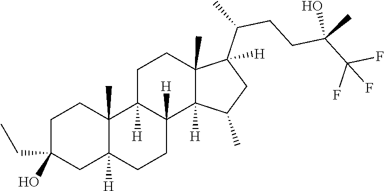 |
| B548 | 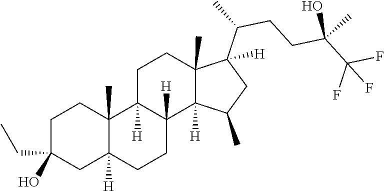 |
| B549 | 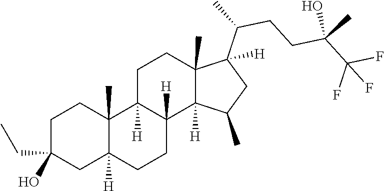 |
| B550 | 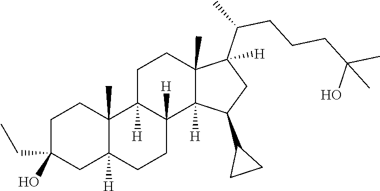 |
| B551 | 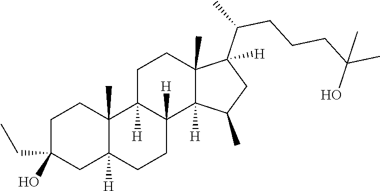 |
| B552 | 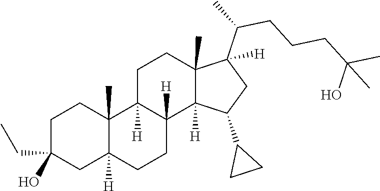 |
| B553 | 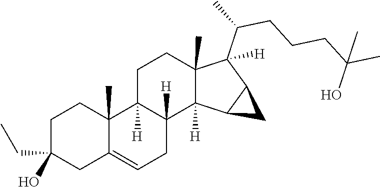 |
| B554 | 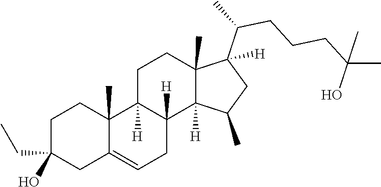 |
| B555 | 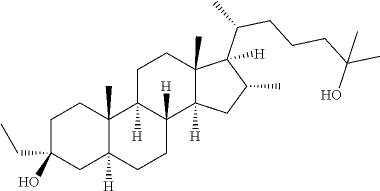 |
| B556 | 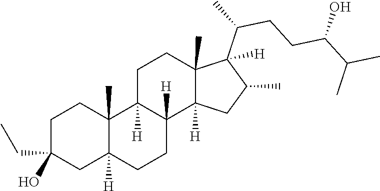 |
| B557 | 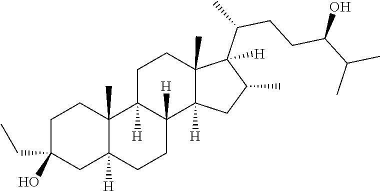 |
| B558 | 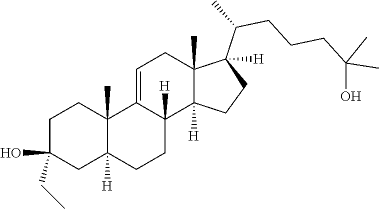 |
| B559 | 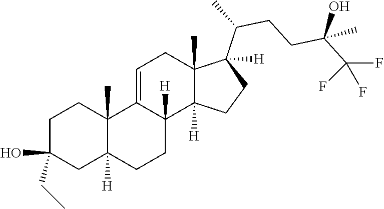 |
| B560 | 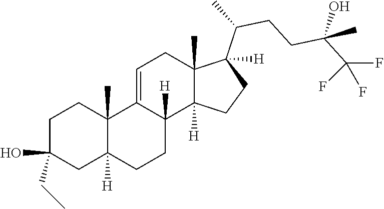 |
| B561 | 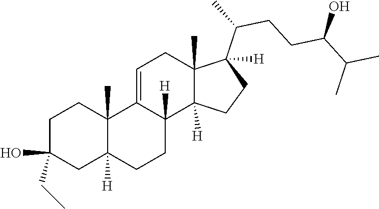 |
| B562 | 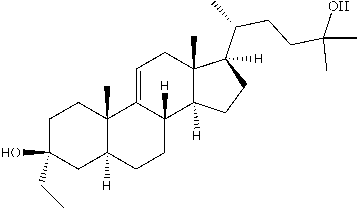 |
| B563 | 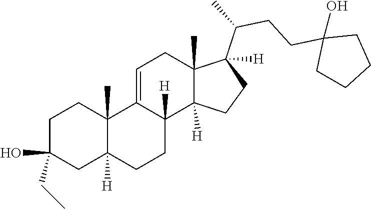 |
| B564 | 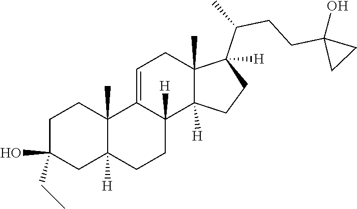 |
| B565 | 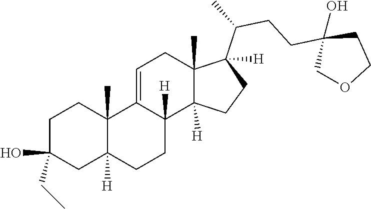 |
| B56 | 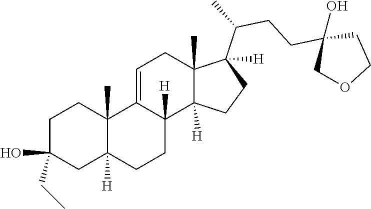 |
| B567 | 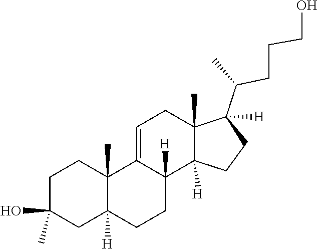 |
| B568 | 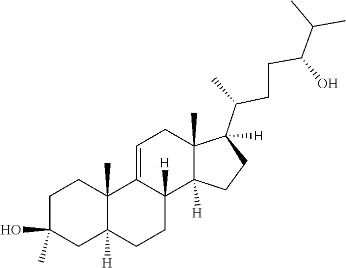 |
| B569 | 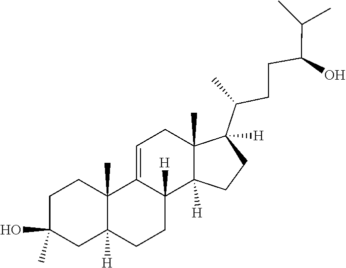 |
| B570 | 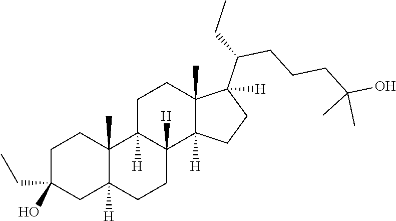 |
| B571 | 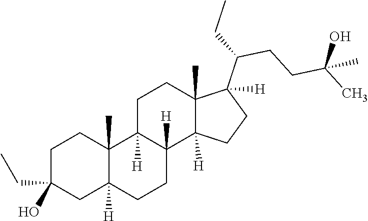 |
| B572 | 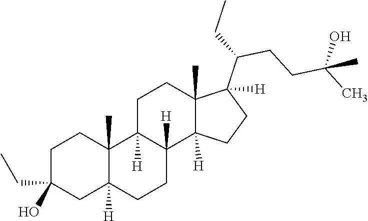 |
| B573 | 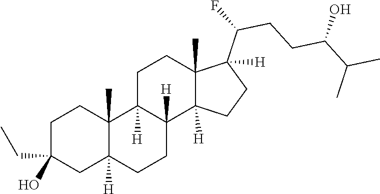 |
| B574 | 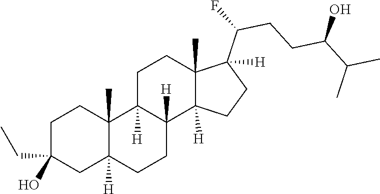 |
| B575 | 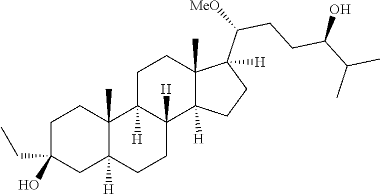 |
| B576 | 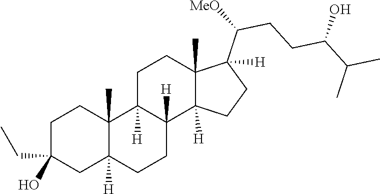 |
| B577 | 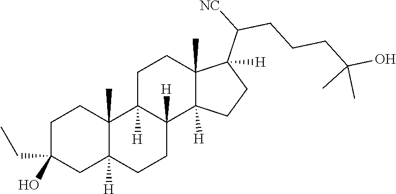 |
| B578 | 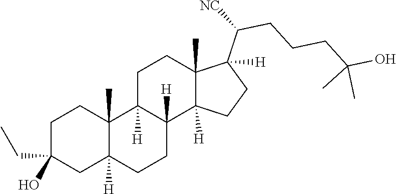 |
| B579 | 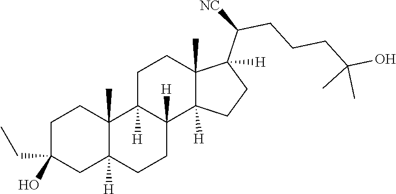 |
| B580 | 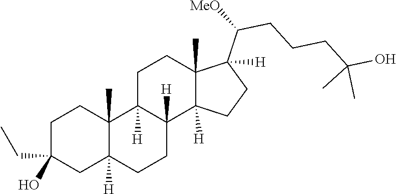 |
| B581 | 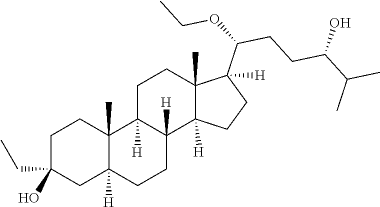 |
| B582 | 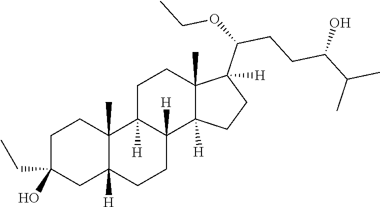 |
| B583 | 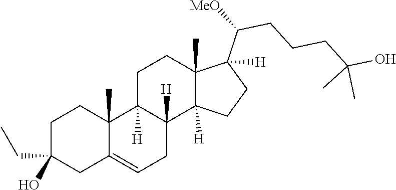 |
| B584 | 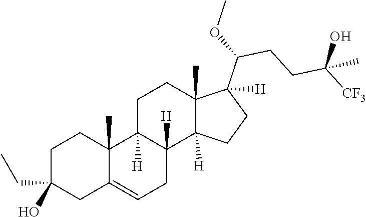 |
| B585 | 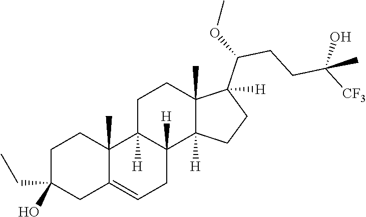 |
| B586 | 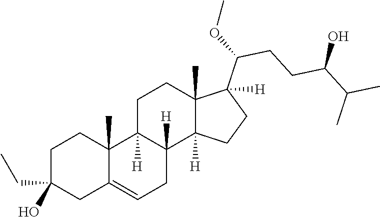 |
| B587 | 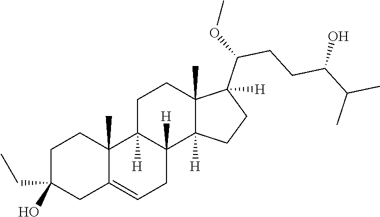 |
| B588 | 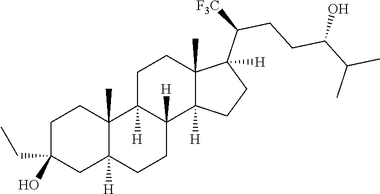 |
| B589 | 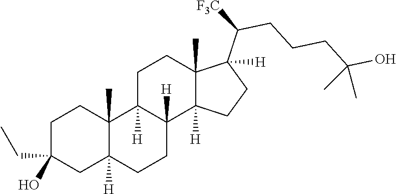 |
| B590 | 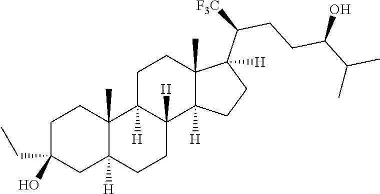 |
| B591 | 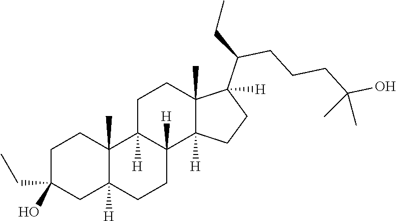 |
| B592 | 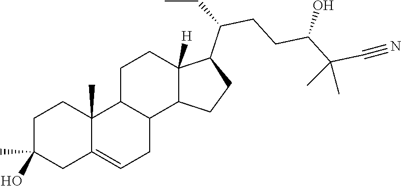 |
| B593 | 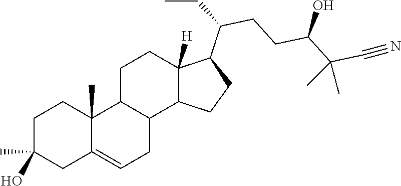 |
| B594 | 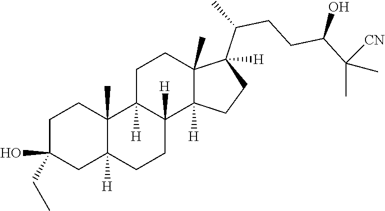 |
| B595 | 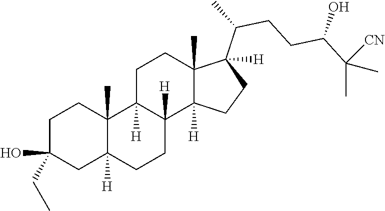 |
| B596 | 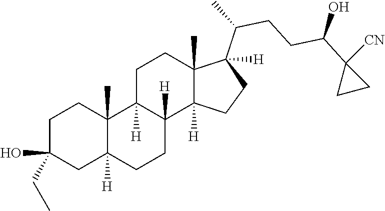 |
| B597 | 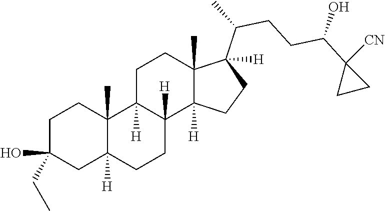 |
| B598 | 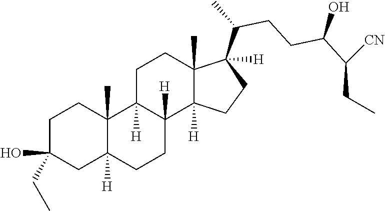 |
| B599 | 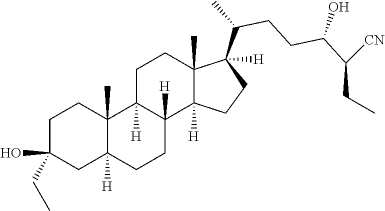 |
| B600 | 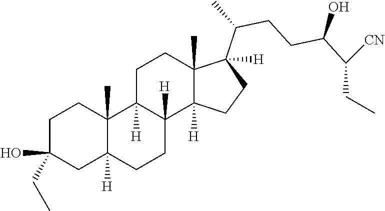 |
| B601 | 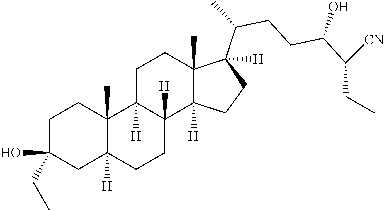 |
| B602 | 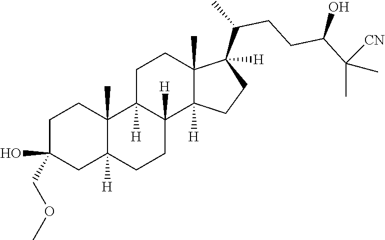 |
| B603 | 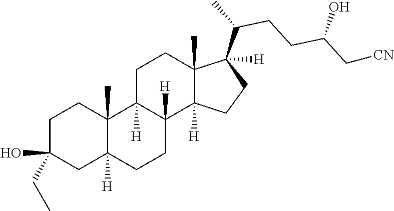 |
| B604 | 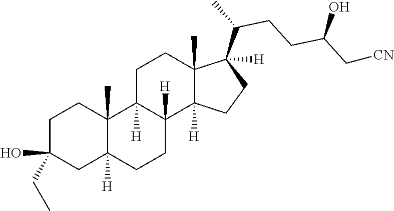 |
| B605 | 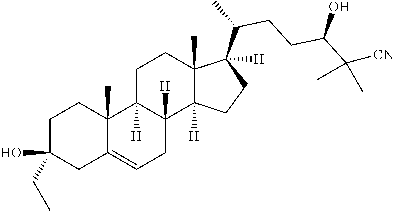 |
| B606 | 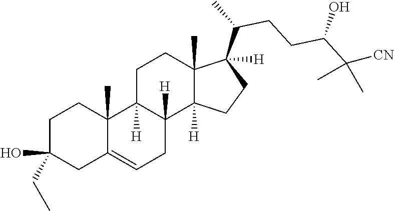 |
| B607 | 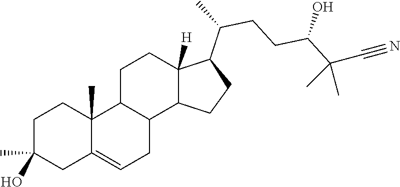 |
| B608 | 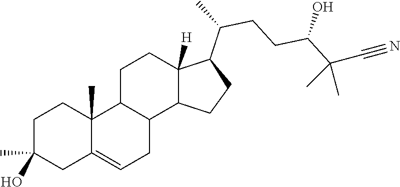 |
| B609 | 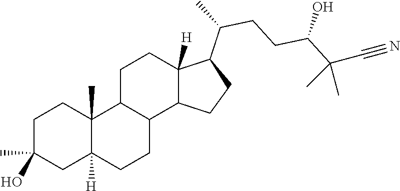 |
| B610 | 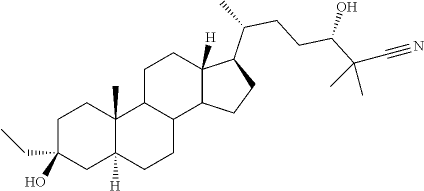 |
| B611 | 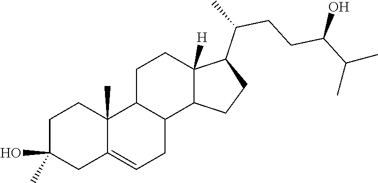 |
| B612 | 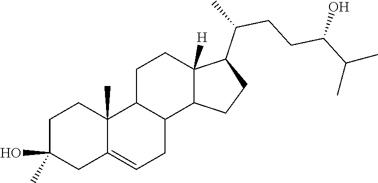 |
| B613 | 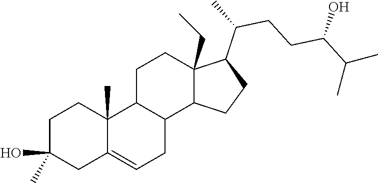 |
| B614 | 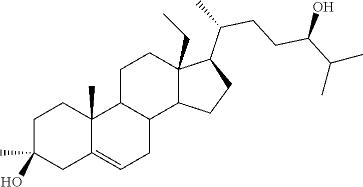 |
| B615 | 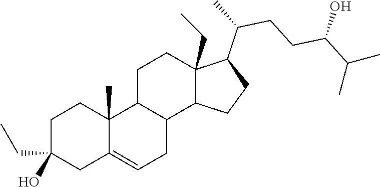 |
| B616 | 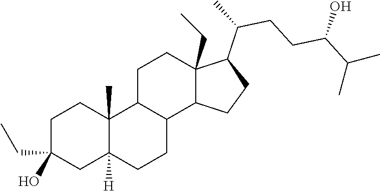 |
| B617 | 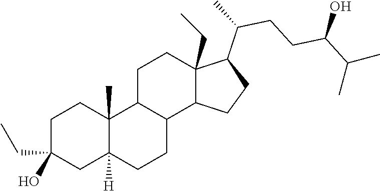 |
| B618 | 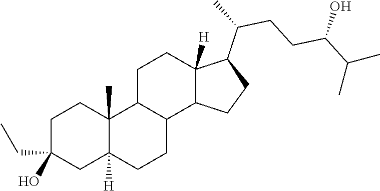 |
| B619 | 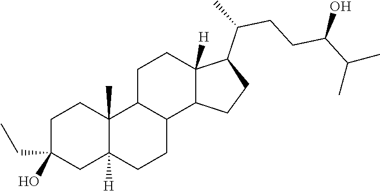 |
| B620 | 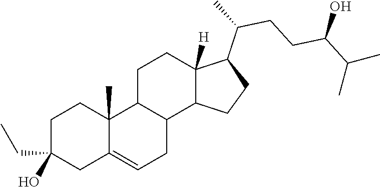 |
| B621 | 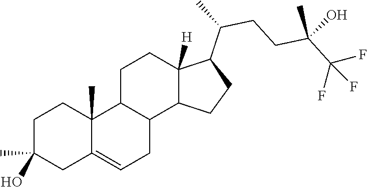 |
| B622 | 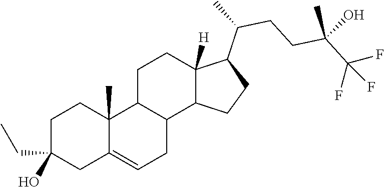 |
| B623 | 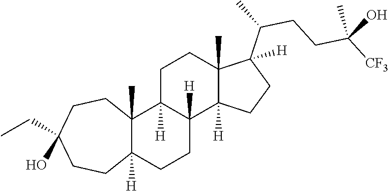 |
| B624 | 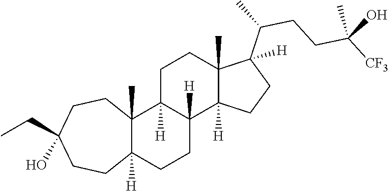 |
| B625 | 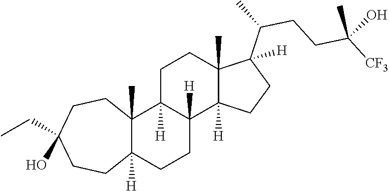 |
| B626 | 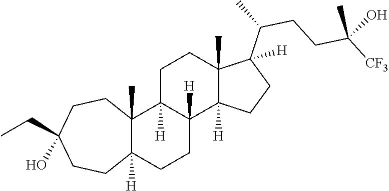 |
| B627 | 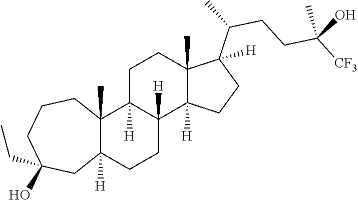 |
| B628 | 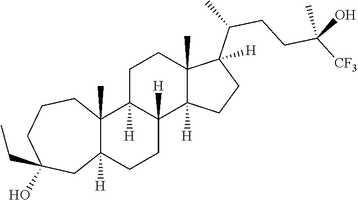 |
| B629 | 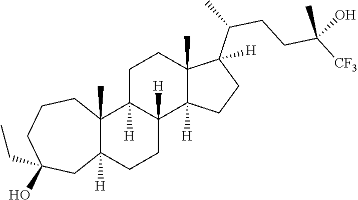 |
| B630 | 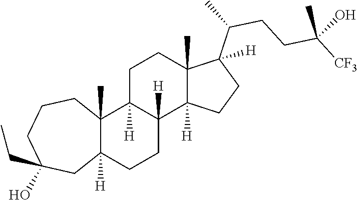 |
| B631 | 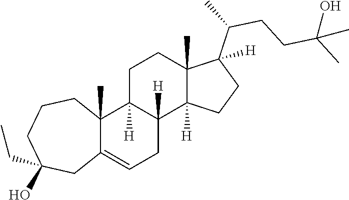 |
| B632 | 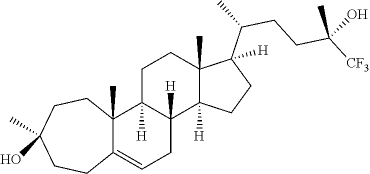 |
| B633 | 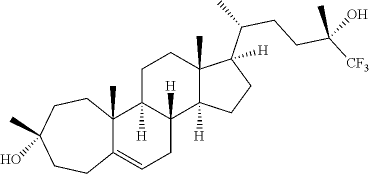 |
| B634 | 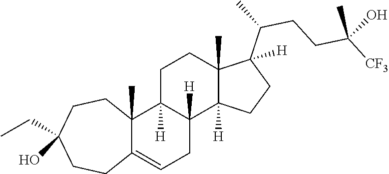 |
| B635 | 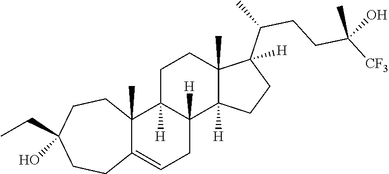 |
| B636 | 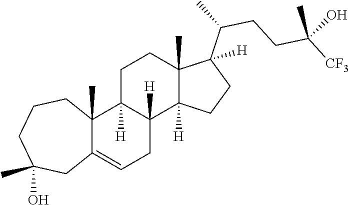 |
| B637 | 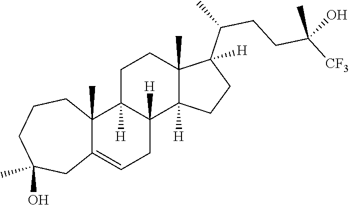 |
| B638 | 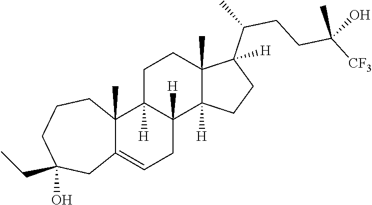 |
| B639 | 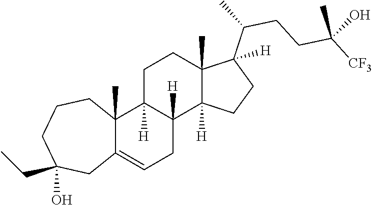 |
| B640 | 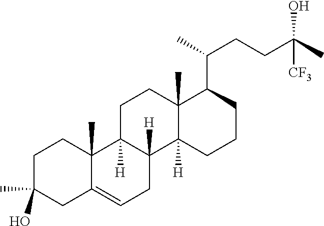 |
| B641 | 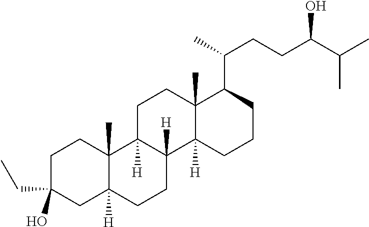 |
| B642 | 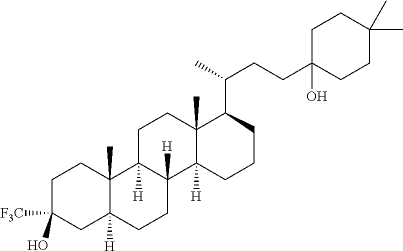 |
| B643 | 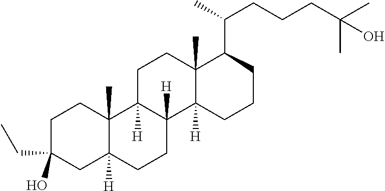 |
| B644 | 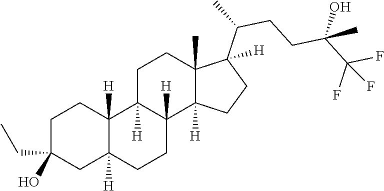 |
| B645 | 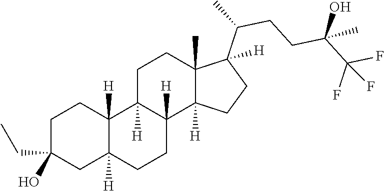 |
| B646 | 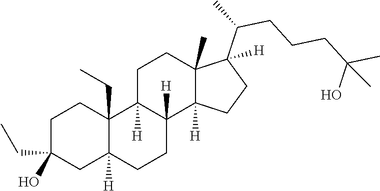 |
| B647 | 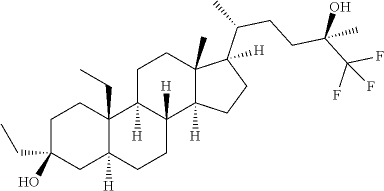 |
| B648 | 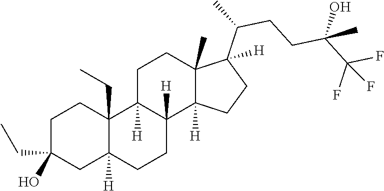 |
| B649 | 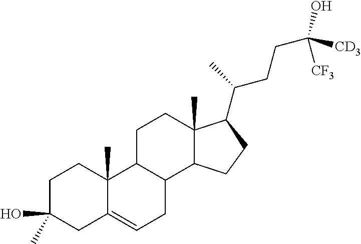 |
| B650 | 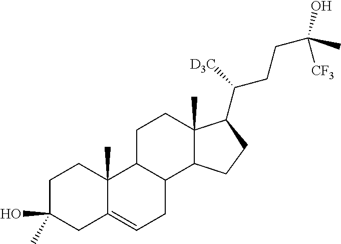 |
| B651 | 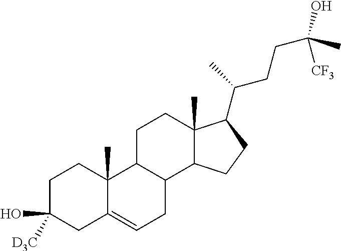 |
| B652 | 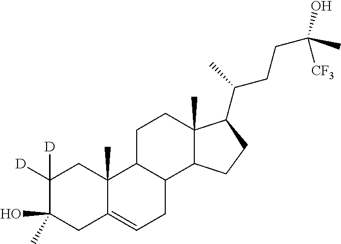 |
| B653 | 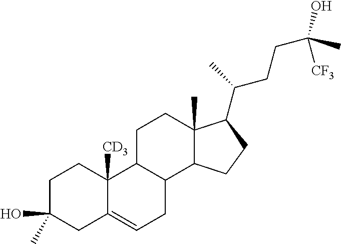 |
| B654 | 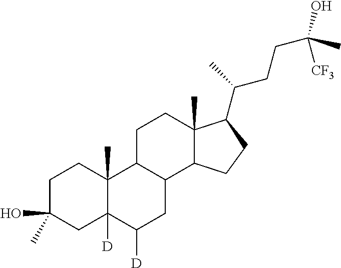 |
| B655 | 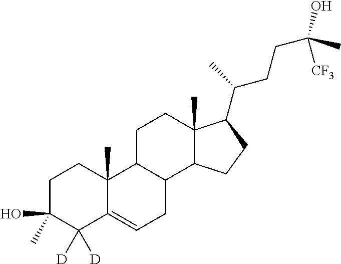 |
| B656 | 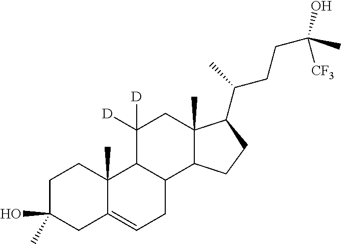 |
| B657 | 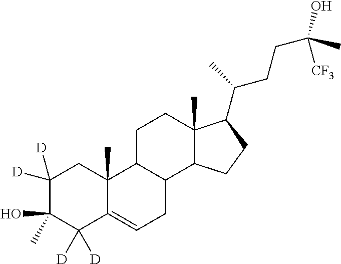 |
| B658 | 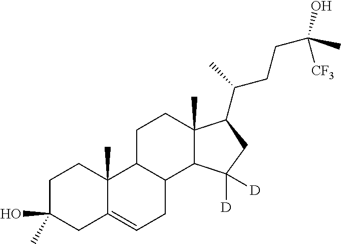 |
| B659 | 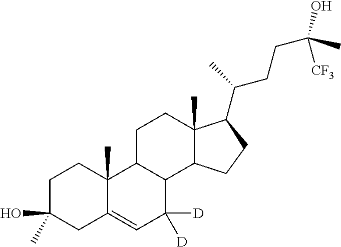 |

In some embodiments, the neuroactive steroid is a compound or pharmaceutically acceptable salt thereof as disclosed in any one of WO2013036835, WO2015195967, WO2014160480, WO2017007832, WO2017007840, WO2017007836, WO2018009867, WO2017193046, WO2017173358, WO2018075699, WO2018064659, U.S. 63/237,126, U.S. 63/237,127, U.S. 63/237,128, U.S. 63/237,129, U.S. 63/237,130, U.S. 63/240,699, U.S. 63/240,700, U.S. 63/240,701, and U.S. 63/247,052 the disclosure of each of which is incorporated by reference herein in their entirety. In some embodiments, the neuroactive steroid is selected from the group consisting of B164, B351 and B411 and pharmaceutically acceptable salts thereof. In some embodiments, the neuroactive steroid is selected from the group consisting of B164, B351 and B411. In some embodiments, the neuroactive steroid is B164 or a pharmaceutically acceptable salt thereof. In some embodiments, the neuroactive steroid is B351 or a pharmaceutically acceptable salt thereof. In some embodiments, the neuroactive steroid is B411 or a pharmaceutically acceptable salt thereof. In some embodiments, the neuroactive steroid is B164. In some embodiments, the neuroactive steroid is B351. In some embodiments, the neuroactive steroid is B411.

The synthesis of compounds B1-B648 is described in WO2013036835, WO2015195967, WO2014160480, WO2017007832, WO2017007840, WO2017007836, WO2018009867, WO2017193046, WO2017173358, WO2018075699, WO2018064659, U.S. 63/237,126, U.S. 63/237,127, U.S. 63/237,128, U.S. 63/237,129, U.S. 63/237,130, U.S. 63/240,699, U.S. 63/240,700, U.S. 63/240,701 and U.S. 63/247,052, the disclosure of each of which is incorporated by reference herein in their entirety.

### Pharmaceutical Compositions

In one aspect, provided herein is a pharmaceutical composition comprising: (a) a muscarinic receptor positive modulator; and (b) an NMDA receptor positive allosteric modulator. In some embodiments, provided herein is a pharmaceutical composition comprising: (a) a muscarinic receptor positive modulator; (b) an NMDA receptor positive allosteric modulator; and (c) a pharmaceutically acceptable carrier. In some embodiments, provided herein is a pharmaceutical composition comprising: (a) a muscarinic receptor agonist; and (b) an NMDA receptor positive allosteric modulator. In some embodiments, provided herein is a pharmaceutical composition comprising: (a) a muscarinic receptor agonist; (b) an NMDA receptor positive allosteric modulator; and (c) a pharmaceutically acceptable carrier. In some embodiments, provided herein is a pharmaceutical composition comprising: (a) a muscarinic receptor positive allosteric modulator; and (b) an NMDA receptor positive allosteric modulator. In some embodiments, provided herein is a pharmaceutical composition comprising: (a) a muscarinic receptor positive allosteric modulator; (b) an NMDA receptor positive allosteric modulator; and (c) a pharmaceutically acceptable carrier. In some embodiments, provided herein is a pharmaceutical composition comprising: (a) a muscarinic receptor partial agonist; and (b) an NMDA receptor positive allosteric modulator. In some embodiments, provided herein is a pharmaceutical composition comprising: (a) a muscarinic receptor partial agonist; (b) an NMDA receptor positive allosteric modulator, and (c) a pharmaceutically acceptable carrier. In some embodiments, provided herein is a pharmaceutical composition comprising: (a) an AChEI; and (b) an NMDA receptor positive allosteric modulator. In some embodiments, provided herein is a pharmaceutical composition comprising: (a) an AChEI; (b) an NMDA receptor positive allosteric modulator, and (c) a pharmaceutically acceptable carrier.

In some embodiments, provided herein is a pharmaceutical composition comprising: (a) an effective amount of a muscarinic receptor positive modulator; and (b) an effective amount of an NMDA receptor positive allosteric modulator. In some embodiments, provided herein is a pharmaceutical composition comprising: (a) an effective amount of a muscarinic receptor agonist; and (b) an effective amount of an NMDA receptor positive allosteric modulator. In some embodiments, provided herein is a pharmaceutical composition comprising: (a) an effective amount of a muscarinic receptor positive allosteric modulator; and (b) an effective amount of an NMDA receptor positive allosteric modulator. In some embodiments, provided herein is a pharmaceutical composition comprising: (a) an effective amount of a muscarinic receptor partial agonist; and (b) an effective amount of an NMDA receptor positive allosteric modulator. In some embodiments, provided herein is a pharmaceutical composition comprising: (a) an effective amount of an AChEI; and (b) an NMDA receptor positive allosteric modulator.

In some embodiments, provided herein is a pharmaceutical composition comprising: (a) an effective amount of a muscarinic receptor positive modulator; (b) an effective amount of an NMDA receptor positive allosteric modulator; and (c) a pharmaceutically acceptable carrier. In some embodiments, provided herein is a pharmaceutical composition comprising: (a) an effective amount of a muscarinic receptor agonist; (b) an effective amount of an NMDA receptor positive allosteric modulator; and (c) a pharmaceutically acceptable carrier. In some embodiments, provided herein is a pharmaceutical composition comprising: (a) an effective amount of a muscarinic receptor positive allosteric modulator; (b) an effective amount of an NMDA receptor positive allosteric modulator; and (c) a pharmaceutically acceptable carrier. In some embodiments, provided herein is a pharmaceutical composition comprising: (a) an effective amount of a muscarinic receptor partial agonist; (b) an effective amount of an NMDA receptor positive allosteric modulator; and (c) a pharmaceutically acceptable carrier. In some embodiments, provided herein is a pharmaceutical composition comprising: (a) an effective amount of an AChEI; (b) an NMDA receptor positive allosteric modulator; and (c) a pharmaceutically acceptable carrier.

In some embodiments, the pharmaceutical composition comprises (a) a therapeutically effective amount of a muscarinic receptor positive modulator; and (b) a therapeutically effective amount of an NMDA receptor positive allosteric modulator. In some embodiments, provided herein is a pharmaceutical composition comprising: (a) a therapeutically effective amount of a muscarinic receptor agonist; and (b) a therapeutically effective amount of an NMDA receptor positive allosteric modulator. In some embodiments, provided herein is a pharmaceutical composition comprising: (a) a therapeutically effective amount of a muscarinic receptor positive allosteric modulator; and (b) a therapeutically effective amount of an NMDA receptor positive allosteric modulator. In some embodiments, provided herein is a pharmaceutical composition comprising: (a) a therapeutically effective amount of a muscarinic receptor partial agonist; and (b) a therapeutically effective amount of an NMDA receptor positive allosteric modulator. In some embodiments, provided herein is a pharmaceutical composition comprising: (a) a therapeutically effective amount of an AChEI; and (b) a therapeutically effective amount of an NMDA receptor positive allosteric modulator.

In some embodiments, the pharmaceutical composition comprises (a) a therapeutically effective amount of a muscarinic receptor positive modulator; (b) a therapeutically effective amount of an NMDA receptor positive allosteric modulator; and (c) a pharmaceutically acceptable carrier. In some embodiments, provided herein is a pharmaceutical composition comprising: (a) a therapeutically effective amount of a muscarinic receptor agonist; (b) a therapeutically effective amount of an NMDA receptor positive allosteric modulator; and (c) a pharmaceutically acceptable carrier. In some embodiments, provided herein is a pharmaceutical composition comprising: (a) a therapeutically effective amount of a muscarinic receptor positive allosteric modulator; (b) a therapeutically effective amount of an NMDA receptor positive allosteric modulator; and (c) a pharmaceutically acceptable carrier. In some embodiments, provided herein is a pharmaceutical composition comprising: (a) a therapeutically effective amount of a muscarinic receptor partial agonist; (b) a therapeutically effective amount of an NMDA receptor positive allosteric modulator; and (c) a pharmaceutically acceptable carrier. In some embodiments, provided herein is a pharmaceutical composition comprising: (a) a therapeutically effective amount of an AChEI; (b) a therapeutically effective amount of an NMDA receptor positive allosteric modulator; and (c) a pharmaceutically acceptable carrier.

The pharmaceutical compositions provided herein can be administered by a variety of routes including, but not limited to, oral (enteral) administration, parenteral (by injection) administration, rectal administration, transdermal administration, intradermal administration, intrathecal administration, subcutaneous (SC) administration, intravenous (IV) administration, intramuscular (IM) administration, and intranasal administration. In some embodiments, the compound or pharmaceutical composition is administered to a subject orally. In some embodiments, the compound or pharmaceutical composition is administered to a subject parenterally. In some embodiments, the compound or pharmaceutical composition is administered to a subject rectally. In some embodiments, the compound or pharmaceutical composition is administered to a subject transdermally. In some embodiments, the compound or pharmaceutical composition is administered to a subject intradermally. In some embodiments, the compound or pharmaceutical composition is administered to a subject intrathecally. In some embodiments, the compound or pharmaceutical composition is administered to a subject subcutaneously. In some embodiments, the compound or pharmaceutical composition is administered to a subject intravenously. In some embodiments, the compound or pharmaceutical composition is administered to a subject intramuscularly. In some embodiments, the compound or pharmaceutical composition is administered to a subject intranasally.

Generally, the compounds (i.e., muscarinic receptor positive modulators and the NMDA PAMs) provided herein are administered in an effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions of the present invention may be further delivered using a variety of dosing methods. For example, in certain embodiments, the pharmaceutical composition may be given as a bolus, e.g., in order to raise the concentration of the compound in the blood to an effective level. The placement of the bolus dose depends on the systemic levels of the active ingredient desired throughout the body, e.g., an intramuscular or subcutaneous bolus dose allows a slow release of the active ingredient, while a bolus delivered directly to the veins (e.g., through an IV drip) allows a much faster delivery which quickly raises the concentration of the active ingredient in the blood to an effective level. In other embodiments, the pharmaceutical composition may be administered as a continuous infusion, e.g., by IV drip, to provide maintenance of a steady-state concentration of the active ingredient in the subject's body. Furthermore, in still yet other embodiments, the pharmaceutical composition may be administered as first as a bolus dose, followed by continuous infusion.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or preferably from about 1 to about 40% by weight) with the remainder being various vehicles or excipients and processing aids helpful for forming the desired dosing form. The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

The compositions may be in sustained release forms or from sustained release drug delivery systems. A description of representative sustained release materials can be found in Remington's Pharmaceutical Sciences.

The present invention also relates to the pharmaceutically acceptable acid addition salt of the compounds (i.e., muscarinic receptor positive modulator inhibitors and the NMDA PAMs) of the disclosure. The acid which may be used to prepare the pharmaceutically acceptable salt is that which forms a non-toxic acid addition salt, i.e., a salt containing pharmacologically acceptable anions such as the hydrochloride, hydroiodide, hydrobromide, nitrate, sulfate, bisulfate, phosphate, acetate, lactate, citrate, tartrate, succinate, maleate, fumarate, benzoate, para-toluenesulfonate, and the like.

### Kits

In one aspect, provided herein is a kit comprising a composition comprising a muscarinic receptor positive modulator (e.g., muscarinic receptor agonist, muscarinic receptor positive allosteric modulator, muscarinic receptor partial agonist, and/or indirect AChEI) as disclosed herein; a composition comprising an NMDA receptor positive allosteric modulator as disclosed herein; and a package insert. In some embodiments, provided herein is a kit comprising a first container, a second container and a package insert, wherein the first container comprises a composition comprising a muscarinic receptor positive modulator (e.g., muscarinic receptor agonist, muscarinic receptor positive allosteric modulator, muscarinic receptor partial agonist, and/or AChEI) as disclosed herein; the second container comprises composition comprising an NMDA receptor PAM as disclosed herein; and the package insert comprises instructions for treating a CNS disease or disorder in a subject. It is understood that the kit may comprise one distinct composition or two or more distinct compositions wherein the first composition comprises a muscarinic receptor positive modulator (e.g., muscarinic receptor agonist, muscarinic receptor positive allosteric modulator, muscarinic receptor partial agonist, and/or AChEI) and second composition comprises an NMDA PAM.

It is understood that the muscarinic receptor positive modulator inhibitor and NMDA PAM compositions may independently have any dosage form. For example, the muscarinic receptor positive modulator composition may be a liquid dosage form, whereas the NMDA PAM composition may be a solid dosage form.

It is understood that the instructions for treating a CNS disease or disorder are intended for treating any one of the diseases or disorders disclosed herein. In some embodiments, the kit comprises instructions for administering the muscarinic receptor positive modulator (e.g., muscarinic receptor agonist, muscarinic receptor positive allosteric modulator, muscarinic receptor partial agonist, and/or AChEI) and the NMDA PAM simultaneously. In some embodiments, the kit comprises instructions for administering the muscarinic receptor positive modulator (e.g., muscarinic receptor agonist, muscarinic receptor positive allosteric modulator, muscarinic receptor partial agonist, and/or AChEI) and the NMDA PAM sequentially. In some embodiments, the kit comprises instructions for administering the muscarinic receptor positive modulator (e.g., muscarinic receptor agonist, muscarinic receptor positive allosteric modulator, muscarinic receptor partial agonist, and/or AChEI) and the NMDA PAM separately. In some embodiments, the kit comprises instructions for administering the muscarinic receptor positive modulator (e.g., muscarinic receptor agonist, muscarinic receptor positive allosteric modulator, muscarinic receptor partial agonist, and/or AChEI) before the NMDA PAM. In some embodiments, the kit comprises instructions for administering the NMDA PAM before the muscarinic receptor positive modulator (e.g., muscarinic receptor agonist, muscarinic receptor positive allosteric modulator, muscarinic receptor partial agonist, and/or AChEI). In some embodiments, the kit comprises instructions for administering the muscarinic receptor positive modulator (e.g., muscarinic receptor agonist, muscarinic receptor positive allosteric modulator, muscarinic receptor partial agonist, and/or AChEI) and the NMDA PAM with the same frequency. In some embodiments, the kit comprises instructions for administering the muscarinic receptor positive modulator (e.g., muscarinic receptor agonist, muscarinic receptor positive allosteric modulator, muscarinic receptor partial agonist, and/or AChEI) and the NMDA PAM with different frequencies.

The kits of the disclosure may be in any suitable packaging. Suitable packaging includes, but is not limited to, vials, bottles, ampoules, jars, syringes, flexible packaging (e.g., sealed Mylar or plastic bags), and the like. Kits may optionally provide additional components such as buffers and interpretive information.

The first and second containers may be of the same or different shape (e.g., vials, syringes and bottles) and/or material (e.g., plastic or glass). The kit may further comprise other materials that may be useful in administering the medicaments, such as diluents, filters, IV bags and lines, needles and syringes.

In some embodiments, the invention provides articles of manufacture comprising contents of the kits described herein.

### Methods of Treatment and Use

The pharmaceutical compositions of the present disclosure are useful for treating various disorders or conditions. In one aspect, the disclosure provides a method of treating a disorder described herein, comprising administering to a subject in need thereof a composition as disclosed herein. In one aspect, the disclosure provides a method for preventing a disorder described herein, comprising administering to a subject in need thereof a composition as disclosed herein.

In one aspect, the disclosure provides a pharmaceutical composition as disclosed herein, for use in treating a disorder described herein. In one aspect, the disclosure provides a pharmaceutical composition as disclosed herein, for use in preventing a disorder described herein.

In one aspect, the disclosure provides the use of a pharmaceutical composition as disclosed herein, for the manufacture of a medicament for treating a disorder described herein. In one aspect, the disclosure provides the use of a pharmaceutical composition as disclosed herein, for the manufacture of a medicament for preventing a disorder described herein.

In one aspect, provided herein are methods of treating a CNS disease or disorder in a subject, comprising administering to the subject a pharmaceutical composition or compounds (i.e., combination of muscarinic receptor positive modulator (e.g., muscarinic receptor agonist, muscarinic receptor positive allosteric modulator, muscarinic receptor partial agonist, and/or indirect-acting (reversible or irreversible) cholinergic agents (e.g., AChEI) and an NMDA receptor positive allosteric modulator) disclosed herein. In some embodiments, provided herein is a method for treating a CNS disease or disorder in a subject, comprising administering to the subject a pharmaceutical composition of the disclosure. In some embodiments, provided herein is a method for treating a CNS disease or disorder in a subject, comprising administering to the subject (a) a muscarinic receptor positive modulator (e.g., muscarinic receptor agonist, muscarinic receptor positive allosteric modulator, muscarinic receptor partial agonist, and/or indirect-acting (reversible or irreversible) cholinergic agents (e.g., AChEI); and (b) an NMDA receptor positive allosteric modulator. In some embodiments, provided herein is a method for treating a CNS disease or disorder in a subject, comprising administering to the subject (a) an effective amount of a muscarinic receptor positive modulator (e.g., muscarinic receptor agonist, muscarinic receptor positive allosteric modulator, muscarinic receptor partial agonist, and/or indirect-acting (reversible or irreversible) cholinergic agents (e.g., AChEI); and (b) an effective amount of an NMDA receptor positive allosteric modulator. In some embodiments, provided herein is a method for treating a CNS disease or disorder in a subject, comprising administering to the subject (a) a therapeutically effective amount of a muscarinic receptor positive modulator (e.g., muscarinic receptor agonist, muscarinic receptor positive allosteric modulator, muscarinic receptor partial agonist, and/or indirect-acting (reversible or irreversible) cholinergic agents (e.g., AChEI); and (b) a therapeutically effective amount of an NMDA receptor positive allosteric modulator.

In another aspect, provided herein are pharmaceutical composition or compounds (i.e., combination of muscarinic receptor positive modulator (e.g., muscarinic receptor agonist, muscarinic receptor positive allosteric modulator, muscarinic receptor partial agonist, and/or indirect-acting (reversible or irreversible) cholinergic agents (e.g., AChEI) and an NMDA receptor positive allosteric) as disclosed herein for use in treating a CNS disease or disorder in a subject. In some embodiments, provided herein is a pharmaceutical composition as disclosed herein for use in treating a CNS disease or disorder in a subject. In some embodiments, provided herein is a combination of muscarinic receptor positive modulator (e.g., muscarinic receptor agonist, muscarinic receptor positive allosteric modulator, muscarinic receptor partial agonist, and/or indirect-acting (reversible or irreversible) cholinergic agents (e.g., AChEI) and an NMDA receptor positive allosteric modulator for use in treating a CNS disease or disorder in a subject. In some embodiments, provided herein is a combination of an effective amount of muscarinic receptor positive modulator (e.g., muscarinic receptor agonist, muscarinic receptor positive allosteric modulator, muscarinic receptor partial agonist, and/or indirect-acting (reversible or irreversible) cholinergic agents (e.g., AChEI) and an effective amount of an NMDA receptor positive allosteric modulator for use in treating a CNS disease or disorder in a subject. In some embodiments, provided herein is a combination of a therapeutically effective amount of muscarinic receptor positive modulator (e.g., muscarinic receptor agonist, muscarinic receptor positive allosteric modulator, muscarinic receptor partial agonist, and/or indirect-acting (reversible or irreversible) cholinergic agents (e.g., AChEI) and a therapeutically effective amount of an NMDA receptor positive allosteric modulator for use in treating a CNS disease or disorder in a subject.

In another aspect, provided herein is the use of a pharmaceutical composition or compounds (i.e., combination of muscarinic receptor positive modulator (e.g., muscarinic receptor agonist, muscarinic receptor positive allosteric modulator, muscarinic receptor partial agonist, and/or indirect-acting (reversible or irreversible) cholinergic agents (e.g., AChEI) and an NMDA receptor positive allosteric modulator) as disclosed herein in the manufacture of a medicament for treating a CNS disease or disorder in a subject. In some embodiments, provided herein is the use of a pharmaceutical composition as disclosed herein in the manufacture of a medicament for treating a CNS disease or disorder in a subject. In some embodiments, provided herein is the use of a combination of muscarinic receptor positive modulator (e.g., muscarinic receptor agonist, muscarinic receptor positive allosteric modulator, muscarinic receptor partial agonist, and/or indirect-acting (reversible or irreversible) cholinergic agents (e.g., AChEI) and an NMDA receptor positive allosteric modulator in the manufacture of a medicament for treating a CNS disease or disorder in a subject. In some embodiments, provided herein is the use of a combination of an effective amount of muscarinic receptor positive modulator (e.g., muscarinic receptor agonist, muscarinic receptor positive allosteric modulator, muscarinic receptor partial agonist, and/or indirect-acting (reversible or irreversible) cholinergic agents (e.g., AChEI) and an effective amount of an NMDA receptor positive allosteric modulator in the manufacture of a medicament for treating a CNS disease or disorder in a subject. In some embodiments, provided herein is the use of a combination of a therapeutically effective amount of muscarinic receptor positive modulator (e.g., muscarinic receptor agonist, muscarinic receptor positive allosteric modulator, muscarinic receptor partial agonist, and/or indirect-acting (reversible or irreversible) cholinergic agents (e.g., AChEI) and a therapeutically effective amount of an NMDA receptor positive allosteric modulator in the manufacture of a medicament for treating a CNS disease or disorder in a subject.

In some embodiments, the disorder is cancer. In some embodiments, the disorder is diabetes. In some embodiments, the disorder is a sterol synthesis disorder. In some embodiments, the disorder is a gastrointestinal (GI) disorder, e.g., constipation, irritable bowel syndrome (IBS), inflammatory bowel disease (IBD) (e.g., ulcerative colitis, Crohn's disease), structural disorders affecting the GI, anal disorders (e.g., hemorrhoids, internal hemorrhoids, external hemorrhoids, anal fissures, perianal abscesses, anal fistula), colon polyps, cancer, colitis. In some embodiments, the disorder is inflammatory bowel disease.

In some embodiments, provided herein are methods of treating a subject suffering from a sterol synthesis disorder or a sterol deficiency disorder, or a CNS-related disorder, comprising administering to the subject a pharmaceutical composition or compounds (i.e., combination of muscarinic receptor positive modulator (e.g., muscarinic receptor agonist, muscarinic receptor positive allosteric modulator, muscarinic receptor partial agonist, and/or indirect-acting (reversible or irreversible) cholinergic agents (e.g., AChEI) and NMDA PAM) disclosed herein. In some embodiments, provided herein is a method of treating a subject suffering from a sterol synthesis disorder or a sterol deficiency disorder, or a CNS-related disorder, comprising administering to the subject a pharmaceutical composition of the disclosure. In some embodiments, provided herein is a method for treating a subject suffering from a sterol synthesis disorder or a sterol deficiency disorder, or a CNS-related disorder, comprising administering to the subject (a) a muscarinic receptor positive modulator (e.g., muscarinic receptor agonist, muscarinic receptor positive allosteric modulator, muscarinic receptor partial agonist, and/or indirect-acting (reversible or irreversible) cholinergic agents (e.g., AChEI); and (b) an NMDA PAM. In some embodiments, provided herein is a method for treating a subject suffering from a sterol synthesis disorder or a sterol deficiency disorder, or a CNS-related disorder, comprising administering to the subject (a) an effective amount of a muscarinic receptor positive modulator (e.g., muscarinic receptor agonist, muscarinic receptor positive allosteric modulator, muscarinic receptor partial agonist, and/or indirect-acting (reversible or irreversible) cholinergic agents (e.g., AChEI); and (b) an effective amount of an NMDA PAM. In some embodiments, provided herein is a method for treating a subject suffering from a sterol synthesis disorder or a sterol deficiency disorder, or a CNS-related disorder, comprising administering to the subject (a) a therapeutically effective amount of a muscarinic receptor positive modulator (e.g., muscarinic receptor agonist, muscarinic receptor positive allosteric modulator, muscarinic receptor partial agonist, and/or indirect-acting (reversible or irreversible) cholinergic agents (e.g., AChEI); and (b) a therapeutically effective amount of an NMDA PAM.

Exemplary CNS conditions related to NMDA-modulation include, but are not limited to, adjustment disorders, anxiety disorders (including obsessive-compulsive disorder, posttraumatic stress disorder, social phobia, generalized anxiety disorder), cognitive disorders (including Alzheimer's disease, cancer-related cognitive impairment, treatment of residual cognitive symptoms, and other forms of dementia (e.g., frontotemporal dementia)), dissociative disorders, eating disorders, mood disorders (including depression (e.g., postpartum depression), bipolar disorder, dysthymic disorder, major depressive disorder, suicidality), schizophrenia or other psychotic disorders (including schizoaffective disorder), sleep disorders (including insomnia), substance abuse-related disorders, personality disorders (including obsessive-compulsive personality disorder), autism spectrum disorders (including those involving mutations to the Shank group of proteins (e.g., Shank3)), neurodevelopmental disorders (including Rett syndrome), multiple sclerosis, sterol synthesis disorders, pain (including acute and chronic pain; headaches, e.g., migraine headaches), encephalopathy secondary to a medical condition (including hepatic encephalopathy and anti-NMDA receptor encephalitis), seizure disorders (including status epilepticus and monogenic forms of epilepsy such as Dravet's disease, and Tuberous Sclerosis Complex (TSC)), stroke, traumatic brain injury, movement disorders (including Huntington's disease and Parkinson's disease) and tinnitus. In certain embodiments, the compound of the present invention, e.g., a compound of Formula (I), and pharmaceutically acceptable salts thereof, can be used to induce sedation or anesthesia. In certain embodiments, the compound described herein (e.g., a compound of Formula (I), and pharmaceutically acceptable salts thereof), is useful in the treatment or prevention of adjustment disorders, anxiety disorders, cognitive disorders, dissociative disorders, eating disorders, mood disorders, schizophrenia or other psychotic disorders, sleep disorders, substance-related disorders, personality disorders, autism spectrum disorders, neurodevelopmental disorders, sterol synthesis disorders, pain, seizure disorders, stroke, traumatic brain injury, movement disorders and vision impairment, hearing loss, and tinnitus. In some embodiments, the disorder is Huntington's disease. In some embodiments, the disorder is Parkinson's disease. In some embodiments, the disorder is an inflammatory disease (e.g., lupus).

In another aspect, provided herein is the use of a pharmaceutical composition or compounds (i.e., combination of muscarinic receptor positive modulator (e.g., muscarinic receptor agonist, muscarinic receptor positive allosteric modulator, muscarinic receptor partial agonist, and/or indirect-acting (reversible or irreversible) cholinergic agents (e.g., AChEI) and NMDA PAM) as disclosed herein in the manufacture of a medicament for treating a subject suffering from a sterol synthesis disorder or a sterol deficiency disorder. In some embodiments, provided herein is the use of a pharmaceutical composition as disclosed herein in the manufacture of a medicament for treating a subject suffering from a sterol synthesis disorder or a sterol deficiency disorder. In some embodiments, provided herein is the use of a combination of muscarinic receptor positive modulator (e.g., muscarinic receptor agonist, muscarinic receptor positive allosteric modulator, muscarinic receptor partial agonist, and/or indirect-acting (reversible or irreversible) cholinergic agents (e.g., AChEI) and an NMDA PAM in the manufacture of a medicament for treating a subject suffering from a sterol synthesis disorder or a sterol deficiency disorder. In some embodiments, provided herein is the use of a combination of an effective amount of muscarinic receptor positive modulator (e.g., muscarinic receptor agonist, muscarinic receptor positive allosteric modulator, muscarinic receptor partial agonist, and/or indirect-acting (reversible or irreversible) cholinergic agents (e.g., AChEI) and an effective amount of an NMDA PAM in the manufacture of a medicament for treating a subject suffering from a sterol synthesis disorder or a sterol deficiency disorder. In some embodiments, provided herein is the use of a combination of a therapeutically effective amount of muscarinic receptor positive modulator (e.g., muscarinic receptor agonist, muscarinic receptor positive allosteric modulator, muscarinic receptor partial agonist, and/or indirect-acting (reversible or irreversible) cholinergic agents (e.g., AChEI) and a therapeutically effective amount of an NMDA PAM in the manufacture of a medicament for treating a subject suffering from a sterol synthesis disorder or a sterol deficiency disorder.

### Diseases and Disorders

Described herein are methods of treating a sterol synthesis disorder. Cholesterol has an essential rule in growth and development. It is a membrane lipid and a precursor to many molecules that play important roles in cellular growth and differentiation, protein glycosylation, and signaling pathways. Biosynthesis of cholesterol involves a number of enzymes and intermediates. Disorders resulting from a deficiency in any of the enzymes involved in cholesterol biosynthesis lead to the accumulation of intermediates and imbalance in biomolecules, resulting in disorders including congenital skeletal malformations, dysmorphic facial features, psychomotor retardation, and failure to thrive. In an embodiment, a sterol synthesis disorder or symptom of a sterol synthesis disorder can be treated by administering to a subject suffering from a sterol synthesis disorder a compound described herein, such as a NMDA receptor modulating compound as described herein. Additional disorders are described below.

### Smith-Lemli-Opitz Syndrome

In one aspect, described herein are methods for treating Smith-Lemli-Opitz Syndrome (or SLOS, or 7-dehydrocholesterol reductase deficiency). SLOS is an inborn error of cholesterol synthesis. In addition to microcephaly, moderate to severe intellectual disability, sensory hypersensitivity, stereotyped behaviors, dysmorphic facial features, and syndactyly of the second/third toes, a feature of the disease is reduced cerebrosterol (24 (S)-hydroxycholesterol) levels. SLOS is an autosomal recessive genetic condition resulting from deficiency in the final enzyme of the cholesterol synthesis pathway and causes low or low-normal plasma cholesterol levels and increased 7- and 8-dehydrocholesterol (DHC; 7DHC and 8DHC) levels. Common therapies currently used include dietary cholesterol supplementation, treatment with 3-hydroxy-3-methylglutaryl coenzyme A reductase inhibitors (HMG CoA reductase inhibitors, also known as statins), and treatment with agents that enhance cholesterol production and/or accretion; and to decrease the accumulation of 7DHC and 8DHC, the potentially toxic precursors of cholesterol.

### Desmosterolosis

Desmosterolosis is a deficiency in desmosterol reductase and has a similar phenotype to SLOS. In one aspect, described herein are methods for treating desmosterolosis.

### Sitosterolemia

Sitosterolemia is a rare autosomal recessive disorder caused by mutations in two ATP-binding cassette (ABC) transporter genes (ABCG5 and ABCG8). Sitosterolemia enhances the absorption of plant sterols and cholesterol from the intestines. Patients typically present with tendon and tuberous xanthomas and premature coronary artery disease. In one aspect, described herein are methods for treating sitosterolemia.

### Cerebrotendinous Xanthomatosis ((TX)

In one aspect, described herein are methods for treating cerebrotendinous xanthomatosis (also referred to as cerebral cholesterosis, or Van Bogaert-Scherer-Epstein syndrome). CTX can be caused by a mutation in the CYP27A1 gene, which produces the sterol 27-hydroxylase enzyme. Sterol 27-hydroxylase metabolizes cholesterol into bile acids (e.g., chenodeoxycholic acid) that are important in the absorption of fats in the intestine. Enzyme dysfunction can lead to cholesterol accumulation in tissues. CTX is characterized by childhood diarrhea, cataracts, tendon xanthomas, reduced mental capability and abnormal movements in adults.

### Mevalonate Kinase Deficiency Syndromes (MKD))

Mevalonate Kinase Deficiency (also referred to as mevalonic aciduria (a more severe form of MKD), or Hyper IgD Syndrome (HIDS, or hyperimmunoglobulinemia D) with period fever syndrome (a more benign form of MKD)) causes an accumulation of mevalonic acid in the urine as a result of insufficient activity of mevalonate kinase. MKD can result in developmental delay, hypotonia, anemia, hepatosplenomegaly, dysmorphic features, mental retardation, and overall failure to thrive. Mevalonic aciduria is characterized by delayed physical and mental development, failure to thrive, recurrent episodes of fever with vomiting and diarrhea, enlarged liver, spleen and lymph nodes, microcephaly (small head size), cataract, low muscle tone, short statute, distinct facial features, ataxia, and anemia. HIDS is characterized by recurrent episodes of fever associated with swollen lymph nodes, joint pain, gastrointestinal issues and skin rash. In one aspect, described herein are methods for treating MKD.

### SC4MOL Gene Mutation (SMO Deficiency)

SC4MOL gene deficiency is a genetic disorder in the cholesterol biosynthesis pathway (e.g., mutations in the SC4MOL gene encoding a novel sterol oxidase). SC$MOL deficiency is characterized by the accumulation of dimethyl and monomethyl sterols that can be detected in blood, skin flakes or primary skin fibroblasts. In one aspect, described herein are methods for treating SMO deficiency.

### Niemann-Pick Disease

Niemann-Pick disease is a lysosomal storage disease resulting from a genetic mutation that affects metabolism. Niemann-Pick disease leads to abnormal accumulation of cholesterol and other fatty substances (lipids) due to an inability of the body to transport the substances. The accumulation damages the affected areas.

### Autism

In one aspect, described herein are methods for treating autism spectrum disorder or autism. Autism spectrum disorder (ASD) and autism refer to a group of complex disorders of brain development. Autism is typically characterized by difficulties in social interaction, for example in verbal and nonverbal communication. Repetitive behaviors are also often seen in individuals having autism. Autism can be associated with intellectual disability, difficulties in motor coordination and attention and physical health issues, e.g., sleep and gastrointestinal disturbances. Individuals having autism can also excel in visual skills, music, math and art. Autism can refer to autistic disorder, childhood disintegrative disorder, pervasive developmental disorder-not otherwise specified (PDD-NOS), and Asperger syndrome. Autism also refers to monogenetic causes of autism such as synaptopathies, e.g., Rett syndrome, Fragile X syndrome, Angelman syndrome.

Disorders Associated with Phenylketonuria

In one aspect, described herein are methods for treating disorders associated with phenylketonuria (e.g., cognitive disorders). Phenylketonuria can lead to hypocholesterolemia and lowered vitamin D status. Total and low-density cholesterols and 25-hydroxy vitamin D have been found to be decreased in subjects suffering from phenylketonuria as compared with subjects not suffering from phenylketonuria ((′lin. Chim. Acta 2013, 416:54-59). 24S-hydroxycholesterol and 27S-hydroxycholesterol and 7a-hydroxycholesterol (e.g., representing peripheral and hepatic cholesterol elimination, respectively) have been shown to be significantly decreased in subjects suffering from phenylketonuria, while 7β-hydroxycholesterol (e.g., reflecting oxidative stress) was increased significantly in subjects suffering from phenylketonuria. Changes in the levels of 24S-OHC and 7β-hydroxycholesterol correlate with phenylalanine level, and 27S-hydroxycholesterol levels may correlate with the 25-hydroxy vitamin D level in subjects suffering from phenylketonuria.

In some embodiments the subject is suffering from a sterol disorder. In some embodiments, the sterol disorder is a sterol synthesis disorder. In some embodiments, the sterol synthesis disorder is a disorder of cholesterol biosynthesis. In some embodiments, the sterol synthesis disorder is characterized by a significant disruption of sterol biosynthesis. In some embodiments, the subject is suffering from a sterol deficiency disorder. In some embodiments the sterol deficiency disorder is characterized by abnormally low levels of sterol. In some embodiments, the sterol deficiency disorder is a 24 (S)-hydroxycholesterol deficiency disorder. In some embodiments, the sterol deficiency disorder is characterized by the presence of 24 (S)-hydroxycholesterol in the plasma of the subject at significantly reduced levels (e.g., at least 1 or 2 standard deviations below) compared with the plasma of a subject not suffering from a sterol deficiency disorder).

In some embodiments, the sterol disorder is selected from: Smith-Lemli-Opitz syndrome; Conradi-Hunermann syndrome; Greenberg dysplasia; Desmosterolosis; Cerebrotendinous Xanthomatosis (CTX); Mevalonate Kinase Deficiency Syndromes (MKD); SC4MOL gene mutation (SMO Deficiency); lathosterolosis; X-linked dominant chondrodysplasia puncata; CHILD syndrome or CK-syndrome; autism spectrum disorder; Niemann-Pick disease; and disorders of dolichol synthesis or metabolism.

In some embodiments, the sterol disorder is Smith-Lemli-Opitz syndrome.

In some embodiments, the sterol disorder is desmosterolosis.

In some embodiments, the sterol disorder is sitosterolemia.

In some embodiments, the sterol disorder is cerebrotendinous xanthomatosis.

In some embodiments, the sterol disorder is mevalonate kinase deficiency syndromes (MKD).

In some embodiments, the sterol disorder is SC4MOL gene deficiency.

In some embodiments, the sterol disorder is Niemann-Pick disease.

In some embodiments, the sterol disorder is an autism spectrum disorder.

In some embodiments, the autism spectrum disorder is autism.

In some embodiments, the sterol disorder are disorders associated with phenylketonuria (e.g., cognitive disorders).

In some embodiments, the muscarinic receptor positive modulator and the NMDA PAM are administered simultaneously. In some embodiments, the muscarinic receptor positive modulator and the NMDA PAM are administered sequentially. In some embodiments, the muscarinic receptor positive modulator and the NMDA PAM are administered separately. In some embodiments, the muscarinic receptor positive modulator is administered before the NMDA PAM. In some embodiments, the NMDA PAM is administered before the muscarinic receptor positive modulator.

In some embodiments, the frequency of administration of the muscarinic receptor positive modulator and the NMDA PAM is the same. In some embodiments, the frequency of administration of the muscarinic receptor positive modulator and the NMDA PAM is different.

### Variations and Modifications

Variations, modifications, and other implementations of what is described herein will be readily apparent to those of ordinary skill in the art without departing from the spirit and scope of the invention. Accordingly, the invention is not to be limited to the preceding description or the following examples.

### OTHER EMBODIMENTS

Certain embodiments of the disclosure are set forth in the following numbered paragraphs:

1. A pharmaceutical composition comprising:
(a) a muscarinic receptor positive modulator; and
(b) an NMDA receptor positive allosteric modulator.

2. The pharmaceutical composition according to embodiment 1, wherein the muscarinic receptor positive modulator is a muscarinic receptor agonist.

3. The pharmaceutical composition according to embodiment 2, wherein the muscarinic receptor agonist is an M1 muscarinic receptor agonist.

4. The pharmaceutical composition according to embodiment 3, wherein the M1 muscarinic receptor agonist is selected from the group consisting of Xanomeline, Cevimeline, AF267B, HTL-9936, AC-260584, AE51090, compounds A1-A5, 77-LH-28-1, EUK1001, RS-86, Talsaclidine, LY-287041 and pharmaceutically acceptable salts thereof.

5. The pharmaceutical composition according to embodiment 2, wherein the muscarinic receptor agonist is an M2 muscarinic receptor agonist.

6. The pharmaceutical composition according to embodiment 5, wherein the M2 muscarinic receptor agonist is selected from the group consisting of Berberine, Arecaidine Propargyl Ester (APE), Oxotremorine, iperoxo and pharmaceutically acceptable salts thereof.

7. The pharmaceutical composition according to embodiment 2, wherein the muscarinic receptor agonist is an M3 muscarinic receptor agonist.

8 The pharmaceutical composition according to embodiment 7, wherein the M3 muscarinic receptor agonist, is selected from the group consisting of acetylcholine, bethanechol, carbachol, L-689660, oxotremorine, pilocarpine, muscarine and pharmaceutically acceptable salts thereof.

9. The pharmaceutical composition according to embodiment 2, wherein the muscarinic receptor agonist is an M4 muscarinic receptor agonist.

10. The pharmaceutical composition according to embodiment 9, wherein the M4 muscarinic receptor agonist is selected from the group consisting of acetylcholine, carbachol. Oxotremorine, Xanomeline, clozapine, EUK1001, and pharmaceutically acceptable salts thereof.

11. The pharmaceutical composition according to embodiment 2, wherein the muscarinic receptor modulator is an M5 muscarinic receptor agonist.

12. The pharmaceutical composition according to embodiment 11, wherein the M5 muscarinic receptor agonist, is selected from the group consisting of Milameline, Sabcomeline, and pharmaceutically acceptable salts thereof.

13. The pharmaceutical composition according to embodiment 1, wherein the muscarinic receptor positive modulator is selected from the group consisting Methacholine, Xanomeline, Cevimeline, AF267B, HTL-9936, AC-260584, AE51090, compounds A1-A5, 77-LH-28-1, Berberine, Arecaidine Propargyl Ester (APE), Oxotremorine, iperoxo, acetylcholine, bethanechol, carbachol, L-689660, Pilocarpine, muscarine, Xanomeline, clozapine, Milameline, Sabcomeline, LY-287041, Talsaclidine, EUK1001, RS-86 and pharmaceutically acceptable salts thereof.

14. The pharmaceutical composition according to embodiment 1, wherein the muscarinic receptor positive modulator is a muscarinic receptor positive allosteric modulator.

15. The pharmaceutical composition according to embodiment 14, wherein the muscarinic receptor positive allosteric modulator is an M1 muscarinic receptor positive allosteric modulator.

16. The pharmaceutical composition according to embodiment 15, wherein the M1 muscarinic receptor positive allosteric modulator is selected from the group consisting of GSK-1034702, TBPB, PQCA, BQCA, BQZ12, MIPS1674, VU0029767, VU0090157, VU0027414, VU0119498, VU0366369, VU0405562, VU0486846, VU0453595, VU0405652, and VU0405645, N-desmethylclozapine, 77-LH-28-1, AC-42, Brucine, ASN-408, TAK-071, 1-(3-[4-butylpiperidino]propyl)-1,3-dihydrobenzimidazol-2-one, compounds A6-A51, MK-7622, and pharmaceutically acceptable salts thereof.

17. The pharmaceutical composition according to embodiment 14, wherein the muscarinic receptor positive allosteric modulator is an M2 muscarinic receptor positive allosteric modulator.

18. The pharmaceutical composition according to embodiment 17, wherein the M2 muscarinic receptor positive allosteric modulator is selected from the group consisting of LY2119620, and pharmaceutically acceptable salts thereof.

19. The pharmaceutical composition according to embodiment 14, wherein the muscarinic receptor positive allosteric modulator is an M3 muscarinic receptor positive allosteric modulator.

20. The pharmaceutical composition according to embodiment 19, wherein the M3 muscarinic receptor allosteric modulator is selected from the group consisting of Amiodarone, N-Ethylamiodarone, and pharmaceutically acceptable salts thereof.

21. The pharmaceutical composition according to embodiment 14, wherein the muscarinic receptor positive allosteric modulator is an M4 muscarinic receptor positive allosteric modulator.

22. The pharmaceutical composition according to embodiment 21, wherein the M4 muscarinic receptor positive allosteric modulator is selected from the group consisting of LY2033298, LY2119620, NMRA-266, VU-0152100, VU00152099, VU0467485, VU10010, and pharmaceutically acceptable salts thereof.

23. The pharmaceutical composition according to embodiment 14, wherein the muscarinic receptor positive allosteric modulator is an M5 muscarinic receptor positive allosteric modulator

24. The pharmaceutical composition according to embodiment 14, wherein the M5 muscarinic receptor allosteric modulatori s selected from the group consisting of ML-380, ML-326, VU00238429, VU0365114, and pharmaceutically acceptable salts thereof.

25. The pharmaceutical composition according to embodiment 1, wherein the muscarinic receptor positive modulator is selected from the group consisting of, GSK-1034702, TBPB, PQCA, BQCA, BQZ12, MIPS1674, VU0029767, VU0090157, VU0027414, VU0119498, VU0366369, VU0405562, VU0486846, VU0453595, VU0405652, and VU0405645, N-desmethylclozapine, 77-LH-28-1, AC-42, Brucine, ASN-408, TAK-071, 1-(3-[4-butylpiperidino]propyl)-1,3-dihydrobenzimidazol-2-one, compounds A6-A51, MK-7622, LY2119620, Amiodarone, N-Ethylamiodarone, LY2033298, VU-0152100, VU00152099, VU0467485, VU10010, ML-380, ML-326, VU00238429, VU0365114_and pharmaceutically acceptable salts thereof.

26. The pharmaceutical composition according to embodiment 1, wherein the muscarinic receptor positive modulator is a muscarinic receptor partial agonist.

27. The pharmaceutical composition according to embodiment 26, wherein the muscarinic receptor partial agonist is an M1 muscarinic receptor partial agonist.

28. The pharmaceutical composition according to embodiment 26, wherein the muscarinic receptor partial agonist is an M2 muscarinic receptor partial agonist.

29. The pharmaceutical composition according to embodiment 26, wherein the muscarinic receptor partial agonist is an M3 muscarinic receptor partial agonist.

30. The pharmaceutical composition according to embodiment 26, wherein the muscarinic receptor partial agonist is an M4 muscarinic receptor partial agonist.

31. The pharmaceutical composition according to embodiment 26, wherein the muscarinic receptor partial agonist is an M5 muscarinic receptor partial agonist.

32. The pharmaceutical composition according to embodiment 26, wherein the muscarinic receptor partial agonist is selected from the group consisting of AF150 (S), SB 202026, (2S,2′R,3'S,5′R)-1-methyl-2-(2-methyl-1,3-oxathiolan-5-yl) pyrrolidine 3-sulfoxide methyl iodide, Iper-8-Naph (N-8-Iper), oxotremorine, Talsaclidine and pilocarpine and pharmaceutically acceptable salts thereof.

33. The pharmaceutical composition according to embodiment 1, wherein the muscarinic receptor positive modulator is selected from the group consisting of Methacholine, Xanomeline, Cevimeline, AF267B, HTL-9936, AC-260584, AE51090, compounds A1-A5, 77-LH-28-1, Berberine, Arecaidine Propargyl Ester (APE), Oxotremorine, iperoxo, acetylcholine, bethanechol, carbachol, L-689660, Pilocarpine, muscarine, LY-287041, RS-86, EUK1001, clozapine, Milameline, Sabcomeline, GSK-1034702, TBPB, PQCA, BQCA, BQZ12, MIPS1674, VU0029767, VU0090157, VU0027414, VU0119498, VU0366369, VU0405562, VU0486846, VU0453595, VU0405652, and VU0405645, N-desmethylclozapine, 77-LH-28-1, AC-42, Brucine, ASN-408, TAK-071, 1-(3-[4-butylpiperidino]propyl)-1,3-dihydrobenzimidazol-2-one, compounds A6-A51, MK-7622, LY2119620, Amiodarone, N-Ethylamiodarone, LY2033298, VU-0152100, VU00152099, VU0467485, VU10010, ML-380, ML-326, VU00238429, VU0365114, AF150 (S), SB 202026, (2S,2′R,3'S,5′R)-1-methyl-2-(2-methyl-1,3-oxathiolan-5-yl) pyrrolidine 3-sulfoxide methyl iodide, Iper-8-Naph (N-8-Iper), oxotremorine, Talsaclidine, and pilocarpine and pharmaceutically acceptable salts thereof.

34. The pharmaceutical composition according to any one of embodiments 1-33, wherein the NMDA receptor positive allosteric modulator is selected from the group consisting of phenanthrene carboxylic acids, naphthoic acids, benzenesulfonamides, thiazoles, pyrrolidinones, tetrahydroisoquinolines, neuroactive steroids, and pharmaceutically acceptable salts thereof.

35. The pharmaceutical composition according to embodiment 34, wherein the NMDA receptor positive allosteric modulator is a phenanthrene or a pharmaceutically acceptable salt thereof.

36. The pharmaceutical composition according to embodiment 35, wherein the phenanthrene is UBP512, and pharmaceutically acceptable salts thereof.

37. The pharmaceutical composition according to embodiment 34, wherein the NMDA receptor positive modulator is a naphthoic acid or a pharmaceutically acceptable salt thereof.

38. The pharmaceutical composition according to embodiment 37, wherein the naphthoic acid is UBP684, and pharmaceutically acceptable salts thereof.

39. The pharmaceutical composition according to embodiment 34, wherein the NMDA receptor positive modulator is a benzenesulfonamide or a pharmaceutically acceptable salt thereof.

40. The pharmaceutical composition according to embodiment 39, wherein the benzenesulfonamide is GNE-9278, and pharmaceutically acceptable salts thereof.

41. The pharmaceutical composition according to embodiment 34, wherein the NMDA receptor positive modulator is a thiazole or a pharmaceutically acceptable salt thereof.

42. The pharmaceutical composition according to embodiment 41, wherein the thiazole is GNE-4376, and pharmaceutically acceptable salts thereof.

43. The pharmaceutical composition according to embodiment 34, wherein the NMDA receptor positive allosteric modulator is a pyrrolidinone or a pharmaceutically acceptable salt thereof.

44. The pharmaceutical composition according to embodiment 43, wherein the pyrrolidinone is selected from a group consisting of PYD-111 and PYD-106, and pharmaceutically acceptable salts thereof.

45. The pharmaceutical composition according to embodiment 34, wherein the NMDA receptor positive allosteric modulator is a tetrahydroisoquinoline, and pharmaceutically acceptable salts thereof.

46. The pharmaceutical composition according to embodiment 45, wherein the tetrahydroisoquinoline is CIQ, and pharmaceutically acceptable salts thereof.

47. The pharmaceutical composition according to embodiment 46, wherein the NMDA receptor positive allosteric modulator is a neuroactive steroid or a pharmaceutically acceptable salt thereof.

48. The pharmaceutical composition according to embodiment 47, wherein the neuroactive steroid is selected from any one of the compounds B1-B659, and pharmaceutically acceptable salts thereof.

49. The pharmaceutical composition according to embodiment 48, wherein the neuroactive steroid is selected from Compounds B164, B351, B411, and pharmaceutically acceptable salts thereof.

50. The pharmaceutical composition according to embodiment 49, wherein the neuroactive steroid is B164, or a pharmaceutically acceptable salt thereof.

51. The pharmaceutical composition according to embodiment 49, wherein the neuroactive steroid is B351, or a pharmaceutically acceptable salt thereof.

52. The pharmaceutical composition according to embodiment 49, wherein the neuroactive steroid is B411, or a pharmaceutically acceptable salt thereof.

53. A kit comprising a first container, a second container and a package insert, wherein:
the first container comprises a composition comprising a muscarinic receptor positive modulator;
the second container comprises composition comprising an NMDA receptor positive allosteric modulator; and
the package insert comprises instructions for treating a CNS disease or disorder in a subject.

54. The kit according to embodiment 53, wherein the muscarinic receptor positive modulator is a muscarinic receptor agonist.

55. The kit according to embodiment 54, wherein the muscarinic receptor agonist is an M1 muscarinic receptor agonist.

56. The kit according to embodiment 55, wherein the M1 muscarinic receptor agonist, is selected from the group consisting of Xanomeline, Cevimeline, AF267B, HTL-9936, AC-260584, AE51090, compounds A1-A5, 77-LH-28-1, EUK 1001, RS-86, Talsaclidine, LY-287041 and pharmaceutically acceptable salts thereof.

57. The kit according to embodiment 54, wherein the muscarinic receptor agonist is an M2 muscarinic receptor agonist.

58. The kit according to embodiment 57, wherein the M2 muscarinic receptor agonist is selected from the group consisting of Berberine, Arecaidine Propargyl Ester (APE), Oxotremorine, iperoxo and pharmaceutically acceptable salts thereof.

59 The kit according to embodiment 54, wherein the muscarinic receptor agonist is an M3 muscarinic receptor agonist.

60. The kit according to embodiment 59, wherein the M3 muscarinic receptor agonist, is selected from the group consisting of acetylcholine, bethanechol, carbachol, L-689660, oxotremorine, pilocarpine, muscarine and pharmaceutically acceptable salts thereof.

61. The kit according to embodiment 54, wherein the muscarinic receptor agonist is an M4 muscarinic receptor agonist.

62. The kit according to embodiment 61, wherein the M4 muscarinic receptor agonist, is selected from the group consisting of acetylcholine, carbachol. Oxotremorine, Xanomeline, clozapine, EUK1001, and pharmaceutically acceptable salts thereof.

63. The kit according to embodiment 54, wherein the muscarinic receptor agonist is an M5 muscarinic receptor agonist.

64. The kit according to embodiment 63, wherein the M5 muscarinic receptor agonist, is selected from the group consisting of Milameline, Sabcomeline, and pharmaceutically acceptable salts thereof.

65. The kit according to embodiment 54, wherein the muscarinic receptor positive modulator is selected from the group consisting Methacholine, Xanomeline, Cevimeline, AF267B, HTL-9936, AC-260584, AE51090, compounds A1-A5, 77-LH-28-1, Berberine, Arecaidine Propargyl Ester (APE), Oxotremorine, iperoxo, acetylcholine, bethanechol, carbachol, L-689660, Pilocarpine, muscarine, clozapine, Milameline, Sabcomeline, LY-287041, Talsaclidine, EUK1001, RS-86 and pharmaceutically acceptable salts thereof.

66. The kit according to embodiment 53, wherein the muscarinic receptor positive modulator is a muscarinic receptor positive allosteric modulator.

67. The kit according to embodiment 66, wherein the muscarinic receptor positive allosteric modulator is an M1 muscarinic receptor positive allosteric modulator.

68. The kit according to embodiment 67, wherein the M1 muscarinic receptor positive allosteric modulator, is selected from the group consisting of GSK-1034702, TBPB, PQCA, BQCA, BQZ12, MIPS1674, VU0029767, VU0090157, VU0027414, VU0119498, VU0366369, VU0405562, VU0486846, VU0453595, VU0405652, and VU0405645, N-desmethylclozapine, 77-LH-28-1, AC-42, Brucine, ASN-408, TAK-071, 1-(3-[4-butylpiperidino]propyl)-1,3-dihydrobenzimidazol-2-one, compounds A6-A51, MK-7622, and pharmaceutically acceptable salts thereof.

69. The kit according to embodiment 66, wherein the muscarinic receptor positive allosteric modulator is an M2 muscarinic receptor positive allosteric modulator.

70 The kit according to embodiment 69, wherein the M2 muscarinic receptor positive allosteric modulator is selected from the group consisting of LY2119620, and pharmaceutically acceptable salts thereof.

71. The kit according to embodiment 66, wherein the muscarinic receptor positive allosteric modulator is an M3 muscarinic receptor positive allosteric modulator.

72. The kit according to embodiment 71, wherein the M3 muscarinic receptor positive allosteric modulator is selected from the group consisting of Amiodarone, N-Ethylamiodarone, and pharmaceutically acceptable salts thereof.

73. The kit according to embodiment 66, wherein the muscarinic receptor positive allosteric modulator is an M4 muscarinic receptor positive allosteric modulator.

74. The kit according to embodiment 73, wherein the M4 muscarinic receptor positive allosteric modulator is selected from the group consisting of LY2033298, LY2119620, NMRA-266, VU-0152100, VU00152099, VU0467485, VU10010 and pharmaceutically acceptable salts thereof.

75. The kit according to embodiment 66, wherein the muscarinic receptor positive allosteric modulator is an M5 muscarinic receptor positive allosteric modulator.

76. The kit according to embodiment 75, wherein the M5 muscarinic receptor positive allosteric modulator, is selected from the group consisting of ML-380, ML-326, VU00238429, and pharmaceutically acceptable salts thereof.

77. The kit according to embodiment 66, wherein the muscarinic receptor positive modulator, is selected from the group consisting of, GSK-1034702, TBPB, PQCA, BQCA, BQZ12, MIPS1674, VU0029767, VU0090157, VU0027414, VU0119498, VU0366369, VU0405562, VU0486846, VU0453595, VU0405652, and VU0405645, N-desmethylclozapine, 77-LH-28-1, AC-42, Brucine, ASN-408, TAK-071, 1-(3-[4-butylpiperidino]propyl)-1,3-dihydrobenzimidazol-2-one, compounds A6-A51, MK-7622, LY2119620, Amiodarone, N-Ethylamiodarone, LY2033298, VU-0152100, VU00152099, VU0467485, VU10010, ML-380, ML-326, VU00238429, VU0365114, and pharmaceutically acceptable salts thereof.

78. The kit according to embodiment 53, wherein the muscarinic receptor positive modulator is a muscarinic receptor partial agonist.

79. The kit according to embodiment 78, wherein the muscarinic receptor partial agonist is an M1 muscarinic receptor partial agonist.

80. The kit according to embodiment 78, wherein the muscarinic receptor partial agonist is an M2 muscarinic receptor partial agonist.

81. The kit according to embodiment 78, wherein the muscarinic receptor partial agonist is an M3 muscarinic receptor partial agonist.

82. The kit according to embodiment 78, wherein the muscarinic receptor partial agonist is an M4 muscarinic receptor partial agonist.

83. The kit according to embodiment 78, wherein the muscarinic receptor partial agonist is an M5 muscarinic receptor partial agonist.

84. The kit according to embodiment 78, AF150 (S), SB 202026, (2S,2′R,3'S,5′R)-1-methyl-2-(2-methyl-1,3-oxathiolan-5-yl) pyrrolidine 3-sulfoxide methyl iodide, Iper-8-Naph (N-8-Iper), talsaclidine, oxotremorine and pilocarpine and pharmaceutically acceptable salts thereof.

85. The kit according to embodiment 53, wherein the muscarinic receptor positive modulator is, selected from the group consisting of Methacholine, Xanomeline, Cevimeline, AF267B, HTL-9936, AC-260584, AE51090, compounds A1-A5, 77-LH-28-1, Berberine, Arecaidine Propargyl Ester (APE), Oxotremorine, iperoxo, acetylcholine, bethanechol, carbachol, L-689660, Pilocarpine, muscarine, clozapine, Milameline, Sabcomeline, LY-287041, Talsaclidine, EUK1001, RS-86 GSK-1034702, TBPB, PQCA, BQCA, BQZ12, MIPS1674, VU0029767, VU0090157, VU0027414, VU0119498, VU0366369, VU0405562, VU0486846, VU0453595, VU0405652, and VU0405645, N-desmethylclozapine, 77-LH-28-1, AC-42, Brucine, ASN-408, TAK-071, 1-(3-[4-butylpiperidino]propyl)-1,3-dihydrobenzimidazol-2-one, compounds A6-A51, MK-7622, LY2119620, Amiodarone, N-Ethylamiodarone, LY2033298, VU-0152100, VU00152099, VU0467485, VU10010, ML-380, ML-326, VU00238429, VU0365114, AF150 (S), SB 202026, (2S,2′R,3'S,5′R)-1-methyl-2-(2-methyl-1,3-oxathiolan-5-yl) pyrrolidine 3-sulfoxide methyl iodide, Iper-8-Naph (N-8-Iper), oxotremorine and pilocarpine and pharmaceutically acceptable salts thereof. 86 The kit according to any one of embodiments 54-85, wherein the NMDA receptor positive allosteric modulator is selected from the group consisting of phenanthrene carboxylic acids, naphthoic acids, benzenesulfonamides, thiazoles, pyrrolidinones, tetrahydroisoquinolines, neuroactive steroids, and pharmaceutically acceptable salts thereof.

87. The kit according to embodiment 86, wherein the NMDA receptor positive allosteric modulator is a phenanthrene or a pharmaceutically acceptable salt thereof.

88. The kit according to embodiment 87, wherein the phenanthrene is UBP512, and pharmaceutically acceptable salts thereof.

89. The kit according to embodiment 86, wherein the NMDA receptor positive allosteric modulator is a naphthoic acid or a pharmaceutically acceptable salt thereof.

90. The kit according to embodiment 89, wherein the naphthoic acid is UBP684, and pharmaceutically acceptable salts thereof.

91. The kit according to embodiment 86, wherein the NMDA receptor positive allosteric modulator is a benzenesulfonamide or a pharmaceutically acceptable salt thereof.

92. The kit according to embodiment 91, wherein the benzenesulfonamide is GNE-9278, and pharmaceutically acceptable salts thereof.

93. The kit according to embodiment 87, wherein the NMDA receptor positive allosteric modulator is a thiazole or a pharmaceutically acceptable salt thereof.

94. The kit according to embodiment 93, wherein the thiazole is GNE-4376, and pharmaceutically acceptable salts thereof.

95. The kit according to embodiment 86, wherein the NMDA receptor positive allosteric modulator is a pyrrolidinone or a pharmaceutically acceptable salt thereof.

96. The kit according to embodiment 94, wherein the pyrrolidinone is selected from a group consisting of PYD-111 and PYD-106, and pharmaceutically acceptable salts thereof.

97. The kit according to embodiment 86, wherein the NMDA receptor positive allosteric modulator is a tetrahydroisoquinoline, and pharmaceutically acceptable salts thereof.

98. The kit according to embodiment 97, wherein the tetrahydroisoquinoline is CIQ, and pharmaceutically acceptable salts thereof.

99. The kit according to embodiment 86, wherein the NMDA receptor positive allosteric modulator is a neuroactive steroid or a pharmaceutically acceptable salt thereof.

100. The kit according to embodiment 99, wherein the neuroactive steroid is selected from any one of the compounds B1-B659, and pharmaceutically acceptable salts thereof.

101. The kit according to embodiment 99, wherein the neuroactive steroid is selected from the group consisting of Compound B164, B351, B411, and pharmaceutically acceptable salts thereof.

102. The kit according to embodiment 101, wherein the neuroactive steroid is B164 or a pharmaceutically acceptable salt thereof.

103. The kit according to embodiment 101, wherein the neuroactive steroid is B351 or a pharmaceutically acceptable salt thereof.

104. The kit according to embodiment 101, wherein the neuroactive steroid is B411 or a pharmaceutically acceptable salt thereof.

105. A method for treating a disorder described herein, comprising administering to a subject in need thereof an effective amount of a muscarinic receptor positive modulator and an NMDA receptor positive allosteric modulator.

106. A method for preventing a disorder described herein, comprising administering to a subject in need thereof an effective amount of a muscarinic receptor positive modulator and an NMDA receptor positive allosteric modulator.

107. The method according to embodiment 105 or 106, wherein the muscarinic receptor positive modulator is a muscarinic receptor agonist.

108. The method according to embodiment 107, wherein the muscarinic receptor agonist is an M1 muscarinic receptor agonist.

109. The method according to embodiment 108, wherein the M1 muscarinic receptor agonist is selected from the group consisting of Xanomeline, Cevimeline, AF267B, HTL-9936, AC-260584, AE51090, compounds A1-A5, 77-LH-28-1, EUK1001, RS-86, Talsaclidine, LY-287041 and pharmaceutically acceptable salts thereof.

110. The method according to embodiment 107, wherein the muscarinic receptor agonist is an M2 muscarinic receptor agonist.

111. The method according to embodiment 110, wherein the M2 muscarinic receptor agonist is selected from the group consisting of Berberine, Arecaidine Propargyl Ester (APE), Oxotremorine, iperoxo and pharmaceutically acceptable salts thereof.

112. The method according to embodiment 107, wherein the muscarinic receptor agonist is an M3 muscarinic receptor agonist.

113. The method according to embodiment 112, wherein the M3 muscarinic receptor agonist, is selected from the group consisting of acetylcholine, bethanechol, carbachol, L-689660, oxotremorine, pilocarpine, muscarine and pharmaceutically acceptable salts thereof.

114. The method according to embodiment 107, wherein the muscarinic receptor agonist is an M4 muscarinic receptor agonist.

115. The method according to embodiment 114, wherein the M4 muscarinic receptor agonist is selected from the group consisting of acetylcholine, carbachol. Oxotremorine, Xanomeline, clozapine, EUK1001_and pharmaceutically acceptable salts thereof.

116. The method according to embodiment 107, wherein the muscarinic receptor modulator is an M5 muscarinic receptor agonist.

117. The method according to embodiment 116, wherein the M5 muscarinic receptor agonist, is selected from the group consisting of Milameline, Sabcomeline, and pharmaceutically acceptable salts thereof.

118. The method according to embodiment 105 or 106, wherein the muscarinic receptor positive modulator is selected from the group consisting Methacholine, Xanomeline, Cevimeline, AF267B, HTL-9936, AC-260584, AE51090, compounds A1-A5, 77-LH-28-1, Berberine, Arecaidine Propargyl Ester (APE), Oxotremorine, iperoxo, acetylcholine, bethanechol, carbachol, L-689660, Pilocarpine, muscarine, clozapine, Milameline, Sabcomeline, LY-287041, Talsaclidine, EUK1001, RS-86 and pharmaceutically acceptable salts thereof.

119. The method according to embodiment 105 or 106, wherein the muscarinic receptor positive modulator is a muscarinic receptor positive allosteric modulator.

120. The method according to embodiment 119, wherein the muscarinic receptor positive allosteric modulator is an M1 muscarinic receptor positive allosteric modulator.

121. The method according to embodiment 120, wherein the M1 muscarinic receptor positive allosteric modulator is selected from the group consisting of GSK-1034702, TBPB, PQCA, BQCA, BQZ12, MIPS1674, VU0029767, VU0090157, VU0027414, VU0119498, VU0366369, VU0405562, VU0486846, VU0453595, VU0405652, and VU0405645, N-desmethylclozapine, 77-LH-28-1, AC-42, Brucine, ASN-408, TAK-071, 1-(3-[4-butylpiperidino]propyl)-1,3-dihydrobenzimidazol-2-one, compounds A6-A51, MK-7622, and pharmaceutically acceptable salts thereof.

122. The method according to embodiment 119, wherein the muscarinic receptor positive allosteric modulator is an M2 muscarinic receptor positive allosteric modulator.

123. The method according to embodiment 122, wherein the M2 muscarinic receptor positive allosteric modulator is selected from the group consisting of LY2119620, and pharmaceutically acceptable salts thereof.

124. The method according to embodiment 119, wherein the muscarinic receptor positive allosteric modulator is an M3 muscarinic receptor positive allosteric modulator.

125. The method according to embodiment 124, wherein the M3 muscarinic receptor allosteric modulator is selected from the group consisting of Amiodarone, N-Ethylamiodarone, and pharmaceutically acceptable salts thereof.

126. The method according to embodiment 119, wherein the muscarinic receptor positive allosteric modulator is an M4 muscarinic receptor positive allosteric modulator.

127. The method according to embodiment 126, wherein the M4 muscarinic receptor positive allosteric modulator is selected from the group consisting of LY2033298, LY2119620, NMRA-266, VU-0152100, VU00152099, VU0467485, VU10010, and pharmaceutically acceptable salts thereof.

128. The method according to embodiment 119, wherein the muscarinic receptor positive allosteric modulator is an M5 muscarinic receptor allosteric modulator.

129. The method according to embodiment 128, wherein the M5 muscarinic receptor positive allosteric modulator is selected from the group consisting of ML-380, ML-326, VU00238429, VU0365114, and pharmaceutically acceptable salts thereof.

130. The method according to embodiment 105 or 106, wherein the muscarinic receptor positive modulator is selected from the group consisting of GSK-1034702, TBPB, PQCA, BQCA, BQZ12, MIPS1674, VU0029767, VU0090157, VU0027414, VU0119498, VU0366369, VU0405562, VU0486846, VU0453595, VU0405652, and VU0405645, N-desmethylclozapine, 77-LH-28-1, AC-42, Brucine, ASN-408, TAK-071, 1-(3-[4-butylpiperidino]propyl)-1,3-dihydrobenzimidazol-2-one, compounds A6-A51, MK-7622, LY2119620, Amiodarone, N-Ethylamiodarone, LY2033298, VU-0152100, VU00152099, VU0467485, VU10010, ML-380, ML-326, VU00238429, VU0365114, and pharmaceutically acceptable salts thereof.

131. The method according to embodiment 105 or 106, wherein the muscarinic receptor positive modulator is a muscarinic receptor partial agonist.

132. The method according to embodiment 131, wherein the muscarinic receptor partial agonist is an M1 muscarinic receptor partial agonist.

133. The method according to embodiment 131, wherein the muscarinic receptor partial agonist is an M2 muscarinic receptor partial agonist.

134. The method according to embodiment 131, wherein the muscarinic receptor partial agonist is an M3 muscarinic receptor partial agonist.

135. The method according to embodiment 131, wherein the muscarinic receptor partial agonist is an M4 muscarinic receptor partial agonist.

136. The method according to embodiment 131, wherein the muscarinic receptor partial agonist is an M5 muscarinic receptor partial agonist.

137. The method according to embodiment 131, wherein the muscarinic receptor partial agonist is selected from the group consisting of AF150 (S), SB 202026, (2S,2′R,3'S,5′R)-1-methyl-2-(2-methyl-1,3-oxathiolan-5-yl) pyrrolidine 3-sulfoxide methyl iodide, Iper-8-Naph (N-8-Iper), talsaclidine, oxotremorine and pilocarpine and pharmaceutically acceptable salts thereof.

138. The method according to embodiment 105 or 106, wherein the muscarinic receptor positive modulator is, selected from the group consisting of Methacholine, Xanomeline, Cevimeline, AF267B, HTL-9936, AC-260584, AE51090, compounds A1-A5, 77-LH-28-1, Berberine, Arecaidine Propargyl Ester (APE), Oxotremorine, iperoxo, acetylcholine, bethanechol, carbachol, L-689660, Pilocarpine, muscarine clozapine, Milameline, Sabcomeline, LY-287041, Talsaclidine, EUK1001, RS-86, GSK-1034702, TBPB, PQCA, BQCA, BQZ12, MIPS1674, VU0029767, VU0090157, VU0027414, VU0119498, VU0366369, VU0405562, VU0486846, VU0453595, VU0405652, and VU0405645, N-desmethylclozapine, 77-LH-28-1, AC-42, Brucine, ASN-408, TAK-071, 1-(3-[4-butylpiperidino]propyl)-1,3-dihydrobenzimidazol-2-one, compounds A6-A51, MK-7622, LY2119620, Amiodarone, N-Ethylamiodarone, LY2033298, VU-0152100, VU00152099, VU0467485, VU10010, ML-380, ML-326, VU00238429, VU0365114, AF150 (S), SB 202026, (2S,2′R,3'S,5′R)-1-methyl-2-(2-methyl-1,3-oxathiolan-5-yl) pyrrolidine 3-sulfoxide methyl iodide, Iper-8-Naph (N-8-Iper), oxotremorine and pilocarpine and pharmaceutically acceptable salts thereof.

139. The method according to any one of embodiments 105-138, wherein the NMDA receptor positive allosteric modulator is selected from the group consisting of phenanthrene carboxylic acids, naphthoic acids, benzenesulfonamides, thiazoles, pyrrolidinones, tetrahydroisoquinolines, neuroactive steroids, and pharmaceutically acceptable salts thereof.

140. The method according to embodiment 139, wherein the NMDA receptor positive allosteric modulator is a phenanthrene or a pharmaceutically acceptable salt thereof.

141. The method according to embodiment 140, wherein the phenanthrene is UBP512, and pharmaceutically acceptable salts thereof.

142. The method according to embodiment 139, wherein the NMDA receptor positive modulator is a naphthoic acid or a pharmaceutically acceptable salt thereof.

143. The method according to embodiment 142, wherein the naphthoic acid is UBP684, and pharmaceutically acceptable salts thereof.

144. The method according to embodiment 139, wherein the NMDA receptor positive modulator is a benzenesulfonamide or a pharmaceutically acceptable salt thereof.

145. The method according to embodiment 144, wherein the benzenesulfonamide is GNE-9278, and pharmaceutically acceptable salts thereof.

146. The method according to embodiment 139, wherein the NMDA receptor positive modulator is a thiazole or a pharmaceutically acceptable salt thereof.

147. The method according to embodiment 146, wherein the thiazole is GNE-4376, and pharmaceutically acceptable salts thereof.

148. The method according to embodiment 139, wherein the NMDA receptor positive allosteric modulator is a pyrrolidinone or a pharmaceutically acceptable salt thereof.

149. The method according to embodiment 148, wherein the pyrrolidinone is selected from a group consisting of PYD-111 and PYD-106, and pharmaceutically acceptable salts thereof.

150. The method according to embodiment 139, wherein the NMDA receptor positive allosteric modulator is a tetrahydroisoquinoline, and pharmaceutically acceptable salts thereof.

151. The method according to embodiment 150, wherein the tetrahydroisoquinoline is CIQ, and pharmaceutically acceptable salts thereof.

152. The method according to embodiment 151, wherein the NMDA receptor positive allosteric modulator is a neuroactive steroid or a pharmaceutically acceptable salt thereof. 153. The method according to embodiment 152, wherein the neuroactive steroid is selected from any one of the compounds B1-B659, and pharmaceutically acceptable salts thereof.

154. The method according to embodiment 153, wherein the neuroactive steroid is selected from Compound B164, B351, B411, and pharmaceutically acceptable salts thereof. 155. The method according to embodiment 154, wherein the neuroactive steroid is B164, or a pharmaceutically acceptable salt thereof.

156. The method according to embodiment 154, wherein the neuroactive steroid is B351, or a pharmaceutically acceptable salt thereof.

157. The method according to embodiment 154, wherein the neuroactive steroid is B411, or a pharmaceutically acceptable salt thereof.

158. The method according to any one of embodiments 105-157, wherein the disorder is a gastrointestinal (GI) disorder e.g., constipation, irritable bowel syndrome (IBS), inflammatory bowel disease (IBD) (e.g., ulcerative colitis, Crohn's disease), structural disorders affecting the GI, anal disorders (e.g., hemorrhoids, internal hemorrhoids, external hemorrhoids, anal fissures, perianal abscesses, anal fistula), colon polyps, cancer, colitis.

159. The method according to embodiment 158, wherein the disorder is inflammatory bowel disease.

160. The method according to any one of embodiments 105-157, wherein the disorder is cancer, diabetes, or a sterol synthesis disorder.

161. The method according to embodiment 160, wherein the disorder is a sterol synthesis disorder.

162. The method according to any one of embodiments 105-157, wherein the disorder is a CNS-related condition.

163. The method according to embodiment 162, wherein the CNS-related condition is an adjustment disorder, anxiety disorder (including obsessive-compulsive disorder, posttraumatic stress disorder, and social phobia), cognitive disorder (including Alzheimer's disease, cancer-related cognitive impairment, treatment of residual cognitive symptoms, and other forms of dementia (e.g., frontotemporal dementia)), delirium, dissociative disorder, eating disorder, mood disorder (including depression (e.g., postpartum depression), bipolar disorder, dysthymic disorder, major depressive disorder, suicidality), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins (e.g., Shank3)), neurodevelopmental disorder (including Rett syndrome, Tuberous Sclerosis complex), multiple sclerosis, pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease), vision impairment, hearing loss, and tinnitus.

164. The method according to any one of embodiments 105-163, wherein the muscarinic receptor positive modulator and the NMDA receptor positive allosteric modulator are administered simultaneously.

165. The method according to any one of embodiments 105-163, wherein the muscarinic receptor positive modulator and the NMDA receptor positive allosteric modulator are administered sequentially.

166. The method according to any one of embodiments 105-163, wherein the muscarinic receptor positive modulator and the NMDA receptor positive allosteric modulator are administered separately.

167. The method according to embodiments 165 or 166, wherein the muscarinic receptor positive modulator is administered before the NMDA receptor positive allosteric modulator. 168. The method according to embodiments 165 or 166, wherein the NMDA receptor positive allosteric modulator is administered before the muscarinic receptor positive modulator.

169. The method according to any one of embodiments 105-168, wherein the frequency of administration of the muscarinic receptor positive modulator and the NMDA receptor positive allosteric modulator is the same.

170. The method according to any one of embodiments 105-168, wherein the frequency of administration of the muscarinic receptor positive modulator and the NMDA receptor positive allosteric modulator is different.

171. A pharmaceutical composition according to any one of embodiments 1-52 for use in treating a disorder described herein.

172. A pharmaceutical composition according to any one of embodiments 1-52 for use in preventing a disorder described herein.

173. The pharmaceutical composition for use according to embodiment 171 or 172, wherein the disorder is a gastrointestinal (GI) disorder e.g., constipation, irritable bowel syndrome (IBS), inflammatory bowel disease (IBD) (e.g., ulcerative colitis, Crohn's disease), structural disorders affecting the GI, anal disorders (e.g., hemorrhoids, internal hemorrhoids, external hemorrhoids, anal fissures, perianal abscesses, anal fistula), colon polyps, cancer, colitis.

174. The pharmaceutical composition for use according to embodiment 173, wherein the disorder is inflammatory bowel disease.

175. The pharmaceutical composition for use according to embodiment 171 or 172, wherein the disorder is cancer, diabetes, or a sterol synthesis disorder.

176. The pharmaceutical composition for use according to embodiment 175, wherein the disorder is a sterol synthesis disorder.

177. The pharmaceutical composition for use according to embodiment 171 or 172, wherein the disorder is a CNS-related condition.

178. The pharmaceutical composition for use according to embodiment 177, wherein the CNS-related condition is an adjustment disorder, anxiety disorder (including obsessive-compulsive disorder, posttraumatic stress disorder, and social phobia), cognitive disorder (including Alzheimer's disease, cancer-related cognitive impairment, treatment of residual cognitive symptoms, and other forms of dementia (e.g., frontotemporal dementia)), delirium, dissociative disorder, eating disorder, mood disorder (including depression (e.g., postpartum depression), bipolar disorder, dysthymic disorder, major depressive disorder, suicidality), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins (e.g., Shank3)), neurodevelopmental disorder (including Rett syndrome, Tuberous Sclerosis complex), multiple sclerosis, pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease), vision impairment, hearing loss, and tinnitus.

179. Use of a pharmaceutical composition according to any one of embodiments 1-52 for the manufacture of a medicament for treating a disorder described herein.

180. Use of a pharmaceutical composition according to any one of embodiments 1-52 for the manufacture of a medicament for preventing a disorder described herein.

181. The use according to embodiment 179 or 180, wherein the disorder is a gastrointestinal (GI) disorder e.g., constipation, irritable bowel syndrome (IBS), inflammatory bowel disease (IBD) (e.g., ulcerative colitis, Crohn's disease), structural disorders affecting the GI, anal disorders (e.g., hemorrhoids, internal hemorrhoids, external hemorrhoids, anal fissures, perianal abscesses, anal fistula), colon polyps, cancer, colitis.

182. The use according to embodiment 181, wherein the disorder is inflammatory bowel disease.

183. The use according to embodiment 179 or 180, wherein the disorder is cancer, diabetes, or a sterol synthesis disorder.

184. The use according to embodiment 183, wherein the disorder is a sterol synthesis disorder.

185. The use according to embodiment 179 or 180, wherein the disorder is a CNS-related condition.

186. The use according to embodiment 185, wherein the CNS-related condition is an adjustment disorder, anxiety disorder (including obsessive-compulsive disorder, posttraumatic stress disorder, and social phobia), cognitive disorder (including Alzheimer's disease, cancer-related cognitive impairment, treatment of residual cognitive symptoms, and other forms of dementia (e.g., frontotemporal dementia)), delirium, dissociative disorder, eating disorder, mood disorder (including depression (e.g., postpartum depression), bipolar disorder, dysthymic disorder, major depressive disorder, suicidality), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins (e.g., Shank3)), neurodevelopmental disorder (including Rett syndrome, Tuberous Sclerosis complex), multiple sclerosis, pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease), vision impairment, hearing loss, and tinnitus.

What is claimed is:

1. A pharmaceutical composition comprising:
(a) a muscarinic receptor positive modulator; and
(b) an NMDA receptor positive allosteric modulator.

2. The pharmaceutical composition according to claim 1, wherein the muscarinic receptor positive modulator is a muscarinic receptor agonist.

3. The pharmaceutical composition according to claim 1, wherein the muscarinic receptor positive modulator is a muscarinic receptor positive allosteric modulator.

4. The pharmaceutical composition according to claim 1, wherein the muscarinic receptor positive modulator is a muscarinic receptor partial agonist.

5. A kit comprising a first container, a second container and a package insert, wherein:
the first container comprises a composition comprising a muscarinic receptor positive modulator;
the second container comprises composition comprising an NMDA receptor positive allosteric modulator; and
the package insert comprises instructions for treating a CNS disease or disorder in a subject.

6. The kit according to claim 5, wherein the muscarinic receptor positive modulator is a muscarinic receptor agonist.

7. The kit according to claim 5, wherein the muscarinic receptor positive modulator is a muscarinic receptor positive allosteric modulator.

8. The kit according to claim 5, wherein the muscarinic receptor positive modulator is a muscarinic receptor partial agonist.

9. A method for treating a disorder described herein, comprising administering to a subject in need thereof an effective amount of the pharmaceutical composition according to any one of claims 1-4.

10. A method for preventing a disorder described herein, comprising administering to a subject in need thereof an effective amount of the pharmaceutical composition according to any one of claims 1-4.

11. The method according to any one of claims 9-10, wherein the disorder is a gastrointestinal (GI) disorder e.g., constipation, irritable bowel syndrome (IBS), inflammatory bowel disease (IBD) (e.g., ulcerative colitis, Crohn's disease), structural disorders affecting the GI, anal disorders (e.g., hemorrhoids, internal hemorrhoids, external hemorrhoids, anal fissures, perianal abscesses, anal fistula), colon polyps, cancer, colitis.

12. The method according to claim 11, wherein the disorder is inflammatory bowel disease.

13. The method according to any one of claim 9-10, wherein the disorder is cancer, diabetes, or a sterol synthesis disorder.

14. The method according to claim 13, wherein the disorder is a sterol synthesis disorder.

15. The method according to any one of claims 9-10, wherein the disorder is a CNS-related condition.

16. The method according to claim 15, wherein the CNS-related condition is an adjustment disorder, anxiety disorder (including obsessive-compulsive disorder, posttraumatic stress disorder, and social phobia), cognitive disorder (including Alzheimer's disease, cancer-related cognitive impairment, treatment of residual cognitive symptoms, and other forms of dementia (e.g., frontotemporal dementia)), delirium, dissociative disorder, eating disorder, mood disorder (including depression (e.g., postpartum depression), bipolar disorder, dysthymic disorder, major depressive disorder, suicidality), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins (e.g., Shank3)), neurodevelopmental disorder (including Rett syndrome, Tuberous Sclerosis complex), multiple sclerosis, pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease), vision impairment, hearing loss, and tinnitus.

17. The method according to any one of claims 9-16, wherein the muscarinic receptor positive modulator and the NMDA receptor positive allosteric modulator are administered simultaneously.

18. The method according to any one of claims 9-16, wherein the muscarinic receptor positive modulator and the NMDA receptor positive allosteric modulator are administered sequentially.

19. The method according to any one of claims 9-16, wherein the muscarinic receptor positive modulator and the NMDA receptor positive allosteric modulator are administered separately.

20. The method according to claim 18 or 19, wherein the muscarinic receptor positive modulator is administered before the NMDA receptor positive allosteric modulator.

21. The method according to claim 18 or 19, wherein the NMDA receptor positive allosteric modulator is administered before the muscarinic receptor positive modulator.

22. The method according to any one of claims 9-21, wherein the frequency of administration of the muscarinic receptor positive modulator and the NMDA receptor positive allosteric modulator is the same.

23. The method according to any one of claims 9-21, wherein the frequency of administration of the muscarinic receptor positive modulator and the NMDA receptor positive allosteric modulator is different.

24. A pharmaceutical composition according to any one of claims 1-4 for use in treating a disorder described herein.

25. A pharmaceutical composition according to any one of claims 1-4 for use in preventing a disorder described herein.

26. The pharmaceutical composition for use according to claim 24 or 25, wherein the disorder is a gastrointestinal (GI) disorder e.g., constipation, irritable bowel syndrome (IBS), inflammatory bowel disease (IBD) (e.g., ulcerative colitis, Crohn's disease), structural disorders affecting the GI, anal disorders (e.g., hemorrhoids, internal hemorrhoids, external hemorrhoids, anal fissures, perianal abscesses, anal fistula), colon polyps, cancer, colitis.

27. The pharmaceutical composition for use according to claim 26, wherein the disorder is inflammatory bowel disease.

28. The pharmaceutical composition for use according to claim 24 or 25, wherein the disorder is cancer, diabetes, or a sterol synthesis disorder.

29. The pharmaceutical composition for use according to claim 28, wherein the disorder is a sterol synthesis disorder.

30. The pharmaceutical composition for use according to claim 24 or 25, wherein the disorder is a CNS-related condition.

31. The pharmaceutical composition for use according to claim 30, wherein the CNS-related condition is an adjustment disorder, anxiety disorder (including obsessive-compulsive disorder, posttraumatic stress disorder, and social phobia), cognitive disorder (including Alzheimer's disease, cancer-related cognitive impairment, treatment of residual cognitive symptoms, and other forms of dementia (e.g., frontotemporal dementia)), delirium, dissociative disorder, eating disorder, mood disorder (including depression (e.g., postpartum depression), bipolar disorder, dysthymic disorder, major depressive disorder, suicidality), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins (e.g., Shank3)), neurodevelopmental disorder (including Rett syndrome, Tuberous Sclerosis complex), multiple sclerosis, pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease), vision impairment, hearing loss, and tinnitus.

32. Use of a pharmaceutical composition according to any one of claims 1-4, for the manufacture of a medicament for treating a disorder described herein.

33. Use of a pharmaceutical composition according to any one of claims 1-4, for the manufacture of a medicament for preventing a disorder described herein.

34. The use according to claim 32 or 33, wherein the disorder is a gastrointestinal (GI) disorder e.g., constipation, irritable bowel syndrome (IBS), inflammatory bowel disease (IBD) (e.g., ulcerative colitis, Crohn's disease), structural disorders affecting the GI, anal disorders (e.g., hemorrhoids, internal hemorrhoids, external hemorrhoids, anal fissures, perianal abscesses, anal fistula), colon polyps, cancer, colitis.

35. The use according to claim 34, wherein the disorder is inflammatory bowel disease.

36. The use according to claim 32 or 33, wherein the disorder is cancer, diabetes, or a sterol synthesis disorder.

37. The use according to claim 36, wherein the disorder is a sterol synthesis disorder.

38. The use according to claim 32 or 33, wherein the disorder is a CNS-related condition.

39. The use according to claim 38, wherein the CNS-related condition is an adjustment disorder, anxiety disorder (including obsessive-compulsive disorder, posttraumatic stress disorder, and social phobia), cognitive disorder (including Alzheimer's disease, cancer-related cognitive impairment, treatment of residual cognitive symptoms, and other forms of dementia (e.g., frontotemporal dementia)), delirium, dissociative disorder, eating disorder, mood disorder (including depression (e.g., postpartum depression), bipolar disorder, dysthymic disorder, major depressive disorder, suicidality), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins (e.g., Shank3)), neurodevelopmental disorder (including Rett syndrome, Tuberous Sclerosis complex), multiple sclerosis, pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease), vision impairment, hearing loss, and tinnitus.
